SEQUENCE LISTING (519 sequences)

SEQ ID NO: 1 - protein, 9 aa, Homo sapiens
  PEPTIDE (1)..(9): selected peptide variant with a predicted ability to interact with the TCR and may or can serve as a mammalian tumor vaccine
LQVDQLWDV

SEQ ID NO: 2 - protein, 9 aa, Homo sapiens
  PEPTIDE (1)..(9): selected peptide variant with a predicted ability to interact with the TCR and may or can serve as a mammalian tumor vaccine
RTFCLLVVV

SEQ ID NO: 3 - protein, 9 aa, Homo sapiens
  PEPTIDE (1)..(9): QLREASPWV
QLREASPWV

SEQ ID NO: 4 - protein, 9 aa, Homo sapiens
  PEPTIDE (1)..(9): selected peptide variant with a predicted ability to interact with the TCR and may or can serve as a mammalian tumor vaccine
CLLVVVVVV

SEQ ID NO: 5 - protein, 9 aa, Homo sapiens
  PEPTIDE (1)..(9): selected peptide variant with a predicted ability to interact with the TCR and may or can serve as a mammalian tumor vaccine
FCLLVVVVV

SEQ ID NO: 6 - protein, 9 aa, Homo sapiens
  PEPTIDE (1)..(9): selected peptide variant with a predicted ability to interact with the TCR and may or can serve as a mammalian tumor vaccine
PIYMYSTMV

SEQ ID NO: 7 - protein, 9 aa, Homo sapiens
  PEPTIDE (1)..(9): selected peptide variant with a predicted ability to interact with the TCR and may or can serve as a mammalian tumor vaccine
LVVVVVVFA

SEQ ID NO: 8 - protein, 9 aa, Homo sapiens
  PEPTIDE (1)..(9): selected peptide variant with a predicted ability to interact with the TCR and may or can serve as a mammalian tumor vaccine
TAFWRSLLA

SEQ ID NO: 9 - protein, 9 aa, Homo sapiens
  PEPTIDE (1)..(9): selected peptide variant with a predicted ability to interact with the TCR and may or can serve as a mammalian tumor vaccine
QLWDVLLSR

SEQ ID NO: 10 - protein, 9 aa, Homo sapiens
  PEPTIDE (1)..(9): selected peptide variant with a predicted ability to interact with the TCR and may or can serve as a mammalian tumor vaccine
VQRLPFSTV

SEQ ID NO: 11 - protein, 9 aa, Homo sapiens
  PEPTIDE (1)..(9): selected peptide variant with a predicted ability to interact with the TCR and may or can serve as a mammalian tumor vaccine
PQLRRWLLV

SEQ ID NO: 12 - protein, 9 aa, Homo sapiens
  PEPTIDE (1)..(9): selected peptide variant with a predicted ability to interact with the TCR and may or can serve as a mammalian tumor vaccine
LLVVVVVVF

SEQ ID NO: 13 - protein, 9 aa, Homo sapiens
  PEPTIDE (1)..(9): selected peptide variant with a predicted ability to interact with the TCR and may or can serve as a mammalian tumor vaccine
TFCLLVVVV

SEQ ID NO: 14 - protein, 9 aa, Homo sapiens
  PEPTIDE (1)..(9): selected peptide variant with a predicted ability to interact with the TCR and may or can serve as a mammalian tumor vaccine
GQATPLPVT

SEQ ID NO: 15 - protein, 9 aa, Homo sapiens
  PEPTIDE (1)..(9): selected peptide variant with a predicted ability to interact with the TCR and may or can serve as a mammalian tumor vaccine
TMRPLPGRI

SEQ ID NO: 16 - protein, 9 aa, Homo sapiens
  PEPTIDE (1)..(9): selected peptide variant with a predicted ability to interact with the TCR and may or can serve as a mammalian tumor vaccine
VLLSRELFR

SEQ ID NO: 17 - protein, 9 aa, Homo sapiens
  PEPTIDE (1)..(9): selected peptide variant with a predicted ability to interact with the TCR and may or can serve as a mammalian tumor vaccine
QATPLPVTI

SEQ ID NO: 18 - protein, 9 aa, Homo sapiens
  PEPTIDE (1)..(9): selected peptide variant with a predicted ability to interact with the TCR and may or can serve as a mammalian tumor vaccine
IYMYSTMVF

SEQ ID NO: 19 - protein, 9 aa, Homo sapiens
  PEPTIDE (1)..(9): selected peptide variant with a predicted ability to interact with the TCR and may or can serve as a mammalian tumor vaccine
SDAYPSAFP

SEQ ID NO: 20 - protein, 9 aa, Homo sapiens
  PEPTIDE (1)..(9): selected peptide variant with a predicted ability to interact with the TCR and may or can serve as a mammalian tumor vaccine
RQGRQRRVR

SEQ ID NO: 21 - protein, 9 aa, Homo sapiens
  PEPTIDE (1)..(9): selected peptide variant with a predicted ability to interact with the TCR and may or can serve as a mammalian tumor vaccine
LLRQGRQRR

SEQ ID NO: 22 - protein, 9 aa, Homo sapiens
  PEPTIDE (1)..(9): selected peptide variant with a predicted ability to interact with the TCR and may or can serve as a mammalian tumor vaccine
VGQRIGSVS

SEQ ID NO: 23 - protein, 9 aa, Homo sapiens
  PEPTIDE (1)..(9): selected peptide variant with a predicted ability to interact with the TCR and may or can serve as a mammalian tumor vaccine
VGRSVAIGP

SEQ ID NO: 24 - protein, 9 aa, Homo sapiens
  PEPTIDE (1)..(9): selected peptide variant with a predicted ability to interact with the TCR and may or can serve as a mammalian tumor vaccine
ELHSLWTCD

SEQ ID NO: 25 - protein, 9 aa, Homo sapiens
  PEPTIDE (1)..(9): selected peptide variant with a predicted ability to interact with the TCR and may or can serve as a mammalian tumor vaccine
SPWVRPRRR

SEQ ID NO: 26 - protein, 9 aa, Homo sapiens
  PEPTIDE (1)..(9): selected peptide variant with a predicted ability to interact with the TCR and may or can serve as a mammalian tumor vaccine
PLPGRIEVR

SEQ ID NO: 27 - protein, 9 aa, Homo sapiens
  PEPTIDE (1)..(9): selected peptide variant with a predicted ability to interact with the TCR and may or can serve as a mammalian tumor vaccine
TPEVQGRVP

SEQ ID NO: 28 - protein, 9 aa, Homo sapiens
  PEPTIDE (1)..(9): selected peptide variant with a predicted ability to interact with the TCR and may or can serve as a mammalian tumor vaccine
PWVRPRRRL

SEQ ID NO: 29 - protein, 9 aa, Homo sapiens
  PEPTIDE (1)..(9): selected peptide variant with a predicted ability to interact with the TCR and may or can serve as a mammalian tumor vaccine
VVVVVVFAV

SEQ ID NO: 30 - protein, 9 aa, Homo sapiens
  PEPTIDE (1)..(9): selected peptide variant with a predicted ability to interact with the TCR and may or can serve as a mammalian tumor vaccine
WLLVSSPPS

SEQ ID NO: 31 - protein, 9 aa, Homo sapiens
  PEPTIDE (1)..(9): selected peptide variant with a predicted ability to interact with the TCR and may or can serve as a mammalian tumor vaccine
LVVGRSVAI

SEQ ID NO: 32 - protein, 9 aa, Homo sapiens
  PEPTIDE (1)..(9): selected peptide variant with a predicted ability to interact with the TCR and may or can serve as a mammalian tumor vaccine
RIGSVSFGT

SEQ ID NO: 33 - protein, 9 aa, Homo sapiens
  PEPTIDE (1)..(9): selected peptide variant with a predicted ability to interact with the TCR and may or can serve as a mammalian tumor vaccine
RADLIRLLL

SEQ ID NO: 34 - protein, 9 aa, Homo sapiens
  PEPTIDE (1)..(9): selected peptide variant with a predicted ability to interact with the TCR and may or can serve as a mammalian tumor vaccine
TVGQRIGSV

SEQ ID NO: 35 - protein, 9 aa, Homo sapiens
  PEPTIDE (1)..(9): selected peptide variant with a predicted ability to interact with the TCR and may or can serve as a mammalian tumor vaccine
RTPEVQGRV

SEQ ID NO: 36 - protein, 9 aa, Homo sapiens
  PEPTIDE (1)..(9): selected peptide variant with a predicted ability to interact with the TCR and may or can serve as a mammalian tumor vaccine
RSLLACCQL

SEQ ID NO: 37 - protein, 9 aa, Homo sapiens
  PEPTIDE (1)..(9): selected peptide variant with a predicted ability to interact with the TCR and may or can serve as a mammalian tumor vaccine
YPVQRLPFS

SEQ ID NO: 38 - protein, 9 aa, Homo sapiens
  PEPTIDE (1)..(9): selected peptide variant with a predicted ability to interact with the TCR and may or can serve as a mammalian tumor vaccine
RWLLVSSPP

SEQ ID NO: 39 - protein, 9 aa, Homo sapiens
  PEPTIDE (1)..(9): selected peptide variant with a predicted ability to interact with the TCR and may or can serve as a mammalian tumor vaccine
FWRSLLACC

SEQ ID NO: 40 - protein, 9 aa, Homo sapiens
  PEPTIDE (1)..(9): selected peptide variant with a predicted ability to interact with the TCR and may or can serve as a mammalian tumor vaccine
VVVVFAVCW

SEQ ID NO: 41 - protein, 9 aa, Homo sapiens
  PEPTIDE (1)..(9): selected peptide variant with a predicted ability to interact with the TCR and may or can serve as a mammalian tumor vaccine
TCNSRQAAL

SEQ ID NO: 42 - protein, 9 aa, Homo sapiens
  PEPTIDE (1)..(9): selected peptide variant with a predicted ability to interact with the TCR and may or can serve as a mammalian tumor vaccine
PVQRLPFST

SEQ ID NO: 43 - protein, 9 aa, Homo sapiens
  PEPTIDE (1)..(9): selected peptide variant with a predicted ability to interact with the TCR and may or can serve as a mammalian tumor vaccine
ALSRPGLLR

SEQ ID NO: 44 - protein, 9 aa, Homo sapiens
  PEPTIDE (1)..(9): selected peptide variant with a predicted ability to interact with the TCR and may or can serve as a mammalian tumor vaccine
EPIYMYSTM

SEQ ID NO: 45 - protein, 9 aa, Homo sapiens
  PEPTIDE (1)..(9): selected peptide variant with a predicted ability to interact with the TCR and may or can serve as a mammalian tumor vaccine
VVGRSVAIG

SEQ ID NO: 46 - protein, 9 aa, Homo sapiens
  PEPTIDE (1)..(9): selected peptide variant with a predicted ability to interact with the TCR and may or can serve as a mammalian tumor vaccine
HGRADLIRL

SEQ ID NO: 47 - protein, 9 aa, Homo sapiens
  PEPTIDE (1)..(9): selected peptide variant with a predicted ability to interact with the TCR and may or can serve as a mammalian tumor vaccine
SGVGKSALT

SEQ ID NO: 48 - protein, 9 aa, Homo sapiens
  PEPTIDE (1)..(9): selected peptide variant with a predicted ability to interact with the TCR and may or can serve as a mammalian tumor vaccine
RYPVQRLPF

SEQ ID NO: 49 - protein, 9 aa, Homo sapiens
  PEPTIDE (1)..(9): selected peptide variant with a predicted ability to interact with the TCR and may or can serve as a mammalian tumor vaccine
DLIRLLLKH

SEQ ID NO: 50 - protein, 9 aa, Homo sapiens
  PEPTIDE (1)..(9): selected peptide variant with a predicted ability to interact with the TCR and may or can serve as a mammalian tumor vaccine
ADLIRLLLK

SEQ ID NO: 51 - protein, 9 aa, Homo sapiens
  PEPTIDE (1)..(9): selected peptide variant with a predicted ability to interact with the TCR and may or can serve as a mammalian tumor vaccine
LHSLWTCDC

SEQ ID NO: 52 - protein, 9 aa, Homo sapiens
  PEPTIDE (1)..(9): selected peptide variant with a predicted ability to interact with the TCR and may or can serve as a mammalian tumor vaccine
VAIGPREQW

SEQ ID NO: 53 - protein, 9 aa, Homo sapiens
  PEPTIDE (1)..(9): selected peptide variant with a predicted ability to interact with the TCR and may or can serve as a mammalian tumor vaccine
LIRLLLKHG

SEQ ID NO: 54 - protein, 9 aa, Homo sapiens
  PEPTIDE (1)..(9): selected peptide variant with a predicted ability to interact with the TCR and may or can serve as a mammalian tumor vaccine
SATVTAFWR

SEQ ID NO: 55 - protein, 9 aa, Homo sapiens
  PEPTIDE (1)..(9): selected peptide variant with a predicted ability to interact with the TCR and may or can serve as a mammalian tumor vaccine
GSVSFGTVY

SEQ ID NO: 56 - protein, 9 aa, Homo sapiens
  PEPTIDE (1)..(9): selected peptide variant with a predicted ability to interact with the TCR and may or can serve as a mammalian tumor vaccine
VQGRVPTLE

SEQ ID NO: 57 - protein, 9 aa, Homo sapiens
  PEPTIDE (1)..(9): selected peptide variant with a predicted ability to interact with the TCR and may or can serve as a mammalian tumor vaccine
PQARAVHLP

SEQ ID NO: 58 - protein, 9 aa, Homo sapiens
  PEPTIDE (1)..(9): selected peptide variant with a predicted ability to interact with the TCR and may or can serve as a mammalian tumor vaccine
LSRPGLLRQ

SEQ ID NO: 59 - protein, 9 aa, Homo sapiens
  PEPTIDE (1)..(9): selected peptide variant with a predicted ability to interact with the TCR and may or can serve as a mammalian tumor vaccine
LREASPWVR

SEQ ID NO: 60 - protein, 9 aa, Homo sapiens
  PEPTIDE (1)..(9): selected peptide variant with a predicted ability to interact with the TCR and may or can serve as a mammalian tumor vaccine
RPEVRKTAS

SEQ ID NO: 61 - protein, 9 aa, Homo sapiens
  PEPTIDE (1)..(9): selected peptide variant with a predicted ability to interact with the TCR and may or can serve as a mammalian tumor vaccine
LHGRADLIR

SEQ ID NO: 62 - protein, 9 aa, Homo sapiens
  PEPTIDE (1)..(9): selected peptide variant with a predicted ability to interact with the TCR and may or can serve as a mammalian tumor vaccine
QGRVPTLER

SEQ ID NO: 63 - protein, 9 aa, Homo sapiens
  PEPTIDE (1)..(9): selected peptide variant with a predicted ability to interact with the TCR and may or can serve as a mammalian tumor vaccine
HDPQARAVH

SEQ ID NO: 64 - protein, 9 aa, Homo sapiens
  PEPTIDE (1)..(9): selected peptide variant with a predicted ability to interact with the TCR and may or can serve as a mammalian tumor vaccine
PGLLRQGRQ

SEQ ID NO: 65 - protein, 9 aa, Homo sapiens
  PEPTIDE (1)..(9): selected peptide variant with a predicted ability to interact with the TCR and may or can serve as a mammalian tumor vaccine
IGSVSFGTV

SEQ ID NO: 66 - protein, 9 aa, Homo sapiens
  PEPTIDE (1)..(9): selected peptide variant with a predicted ability to interact with the TCR and may or can serve as a mammalian tumor vaccine
VVVVVFAVC

SEQ ID NO: 67 - protein, 9 aa, Homo sapiens
  PEPTIDE (1)..(9): selected peptide variant with a predicted ability to interact with the TCR and may or can serve as a mammalian tumor vaccine
VHLPELLSL

SEQ ID NO: 68 - protein, 9 aa, Homo sapiens
  PEPTIDE (1)..(9): selected peptide variant with a predicted ability to interact with the TCR and may or can serve as a mammalian tumor vaccine
QLRRWLLVS

SEQ ID NO: 69 - protein, 9 aa, Homo sapiens
  PEPTIDE (1)..(9): selected peptide variant with a predicted ability to interact with the TCR and may or can serve as a mammalian tumor vaccine
GQRIGSVSF

SEQ ID NO: 70 - protein, 9 aa, Homo sapiens
  PEPTIDE (1)..(9): selected peptide variant with a predicted ability to interact with the TCR and may or can serve as a mammalian tumor vaccine
GELHSLWTC

SEQ ID NO: 71 - protein, 9 aa, Homo sapiens
  PEPTIDE (1)..(9): selected peptide variant with a predicted ability to interact with the TCR and may or can serve as a mammalian tumor vaccine
RTMRPLPGR

SEQ ID NO: 72 - protein, 9 aa, Homo sapiens
  PEPTIDE (1)..(9): selected peptide variant with a predicted ability to interact with the TCR and may or can serve as a mammalian tumor vaccine
MYSTMVFLP

SEQ ID NO: 73 - protein, 9 aa, Homo sapiens
  PEPTIDE (1)..(9): selected peptide variant with a predicted ability to interact with the TCR and may or can serve as a mammalian tumor vaccine
TGQATPLPV

SEQ ID NO: 74 - protein, 9 aa, Homo sapiens
  PEPTIDE (1)..(9): selected peptide variant with a predicted ability to interact with the TCR and may or can serve as a mammalian tumor vaccine
AFWRSLLAC

SEQ ID NO: 75 - protein, 9 aa, Homo sapiens
  PEPTIDE (1)..(9): selected peptide variant with a predicted ability to interact with the TCR and may or can serve as a mammalian tumor vaccine
YSTMVFLPW

SEQ ID NO: 76 - protein, 9 aa, Homo sapiens
  PEPTIDE (1)..(9): selected peptide variant with a predicted ability to interact with the TCR and may or can serve as a mammalian tumor vaccine
VDQLWDVLL

SEQ ID NO: 77 - protein, 9 aa, Homo sapiens
  PEPTIDE (1)..(9): selected peptide variant with a predicted ability to interact with the TCR and may or can serve as a mammalian tumor vaccine
RPQLRRWLL

SEQ ID NO: 78 - protein, 9 aa, Homo sapiens
  PEPTIDE (1)..(9): selected peptide variant with a predicted ability to interact with the TCR and may or can serve as a mammalian tumor vaccine
LQLREASPW

SEQ ID NO: 79 - protein, 9 aa, Homo sapiens
  PEPTIDE (1)..(9): selected peptide variant with a predicted ability to interact with the TCR and may or can serve as a mammalian tumor vaccine
HSLWTCDCE

SEQ ID NO: 80 - protein, 9 aa, Homo sapiens
  PEPTIDE (1)..(9): selected peptide variant with a predicted ability to interact with the TCR and may or can serve as a mammalian tumor vaccine
LPGRIEVRT

SEQ ID NO: 81 - protein, 9 aa, Homo sapiens
  PEPTIDE (1)..(9): selected peptide variant with a predicted ability to interact with the TCR and may or can serve as a mammalian tumor vaccine
LWDVLLSRE

SEQ ID NO: 82 - protein, 9 aa, Homo sapiens
  PEPTIDE (1)..(9): selected peptide variant with a predicted ability to interact with the TCR and may or can serve as a mammalian tumor vaccine
EVQGRVPTL

SEQ ID NO: 83 - protein, 9 aa, Homo sapiens
  PEPTIDE (1)..(9): selected peptide variant with a predicted ability to interact with the TCR and may or can serve as a mammalian tumor vaccine
ATVTAFWRS

SEQ ID NO: 84 - protein, 9 aa, Homo sapiens
  PEPTIDE (1)..(9): selected peptide variant with a predicted ability to interact with the TCR and may or can serve as a mammalian tumor vaccine
QVDQLWDVL

SEQ ID NO: 85 - protein, 9 aa, Homo sapiens
  PEPTIDE (1)..(9): selected peptide variant with a predicted ability to interact with the TCR and may or can serve as a mammalian tumor vaccine
ASDAYPSAF

SEQ ID NO: 86 - protein, 9 aa, Homo sapiens
  PEPTIDE (1)..(9): selected peptide variant with a predicted ability to interact with the TCR and may or can serve as a mammalian tumor vaccine
DGLVVGRSV

SEQ ID NO: 87 - protein, 9 aa, Homo sapiens
  PEPTIDE (1)..(9): selected peptide variant with a predicted ability to interact with the TCR and may or can serve as a mammalian tumor vaccine
SGELHSLWT

SEQ ID NO: 88 - protein, 9 aa, Homo sapiens
  PEPTIDE (1)..(9): selected peptide variant with a predicted ability to interact with the TCR and may or can serve as a mammalian tumor vaccine
DQLWDVLLS

SEQ ID NO: 89 - protein, 9 aa, Homo sapiens
  PEPTIDE (1)..(9): selected peptide variant with a predicted ability to interact with the TCR and may or can serve as a mammalian tumor vaccine
FQDHKPKIS

SEQ ID NO: 90 - protein, 9 aa, Homo sapiens
  PEPTIDE (1)..(9): Non-immunogenic peptide used for developing the TCR-binding algorithm
WLLIDTSNA

SEQ ID NO: 91 - protein, 9 aa, Homo sapiens
  PEPTIDE (1)..(9): Non-immunogenic peptide used for developing the TCR-binding algorithm
RVSRPTTVV

SEQ ID NO: 92 - protein, 9 aa, Homo sapiens
  PEPTIDE (1)..(9): Non-immunogenic peptide used for developing the TCR-binding algorithm
YLDLALMSV

SEQ ID NO: 93 - protein, 9 aa, Homo sapiens
  PEPTIDE (1)..(9): Non-immunogenic peptide used for developing the TCR-binding algorithm
FIFLLFLTL

SEQ ID NO: 94 - protein, 9 aa, Homo sapiens
  PEPTIDE (1)..(9): Non-immunogenic peptide used for developing the TCR-binding algorithm
DETGVEVKD

SEQ ID NO: 95 - protein, 9 aa, Homo sapiens
  PEPTIDE (1)..(9): Non-immunogenic peptide used for developing the TCR-binding algorithm
LMLPGMNGI

SEQ ID NO: 96 - protein, 9 aa, Homo sapiens
  PEPTIDE (1)..(9): EMKEGRYEV
EMKEGRYEV

SEQ ID NO: 97 - protein, 9 aa, Homo sapiens
  PEPTIDE (1)..(9): Non-immunogenic peptide used for developing the TCR-binding algorithm
VLLEKATIL

SEQ ID NO: 98 - protein, 9 aa, Homo sapiens
  PEPTIDE (1)..(9): Non-immunogenic peptide used for developing the TCR-binding algorithm
SLLERGQQL

SEQ ID NO: 99 - protein, 9 aa, Homo sapiens
  PEPTIDE (1)..(9): Non-immunogenic peptide used for developing the TCR-binding algorithm
YLSEGDMAA

SEQ ID NO: 100 - protein, 9 aa, Homo sapiens
  PEPTIDE (1)..(9): Non-immunogenic peptide used for developing the TCR-binding algorithm
RVYEALYYV

SEQ ID NO: 101 - protein, 9 aa, Homo sapiens
  PEPTIDE (1)..(9): Non-immunogenic peptide used for developing the TCR-binding algorithm
KLGLLQVTG

SEQ ID NO: 102 - protein, 9 aa, Homo sapiens
  PEPTIDE (1)..(9): Non-immunogenic peptide used for developing the TCR-binding algorithm
SMAGNWAKV

SEQ ID NO: 103 - protein, 9 aa, Homo sapiens
  PEPTIDE (1)..(9): Non-immunogenic peptide used for developing the TCR-binding algorithm
VVWVKITQV

SEQ ID NO: 104 - protein, 9 aa, Homo sapiens
  PEPTIDE (1)..(9): Non-immunogenic peptide used for developing the TCR-binding algorithm
GLYRQWALA

SEQ ID NO: 105 - protein, 9 aa, Homo sapiens
  PEPTIDE (1)..(9): Non-immunogenic peptide used for developing the TCR-binding algorithm
SMGIFLKSL

SEQ ID NO: 106 - protein, 9 aa, Homo sapiens
  PEPTIDE (1)..(9): Non-immunogenic peptide used for developing the TCR-binding algorithm
SLFPEFSEL

SEQ ID NO: 107 - protein, 9 aa, Homo sapiens
  PEPTIDE (1)..(9): Non-immunogenic peptide used for developing the TCR-binding algorithm
GESVPGIEE

SEQ ID NO: 108 - protein, 9 aa, Homo sapiens
  PEPTIDE (1)..(9): Non-immunogenic peptide used for developing the TCR-binding algorithm
YLYVHSPAL

SEQ ID NO: 109 - protein, 9 aa, Homo sapiens
  PEPTIDE (1)..(9): Non-immunogenic peptide used for developing the TCR-binding algorithm
FMKAVCVEV

SEQ ID NO: 110 - protein, 9 aa, Homo sapiens
  PEPTIDE (1)..(9): Non-immunogenic peptide used for developing the TCR-binding algorithm
DSTQTTTQK

SEQ ID NO: 111 - protein, 9 aa, Homo sapiens
  PEPTIDE (1)..(9): Non-immunogenic peptide used for developing the TCR-binding algorithm
YSLEYFQFV

SEQ ID NO: 112 - protein, 9 aa, Homo sapiens
  PEPTIDE (1)..(9): Non-immunogenic peptide used for developing the TCR-binding algorithm
RAKAVRALK

SEQ ID NO: 113 - protein, 9 aa, Homo sapiens
  PEPTIDE (1)..(9): Non-immunogenic peptide used for developing the TCR-binding algorithm
TEQELPQSQ

SEQ ID NO: 114 - protein, 9 aa, Homo sapiens
  PEPTIDE (1)..(9): Non-immunogenic peptide used for developing the TCR-binding algorithm
SLAGFVRML

SEQ ID NO: 115 - protein, 9 aa, Homo sapiens
  PEPTIDE (1)..(9): Non-immunogenic peptide used for developing the TCR-binding algorithm
GLFLTTEAV

SEQ ID NO: 116 - protein, 9 aa, Homo sapiens
  PEPTIDE (1)..(9): Non-immunogenic peptide used for developing the TCR-binding algorithm
RLQSLQTYV

SEQ ID NO: 117 - protein, 9 aa, Homo sapiens
  PEPTIDE (1)..(9): Non-immunogenic peptide used for developing the TCR-binding algorithm
LLPLGYPFV

SEQ ID NO: 118 - protein, 9 aa, Homo sapiens
  PEPTIDE (1)..(9): Non-immunogenic peptide used for developing the TCR-binding algorithm
ALLRQLAEL

SEQ ID NO: 119 - protein, 9 aa, Homo sapiens
  PEPTIDE (1)..(9): Non-immunogenic peptide used for developing the TCR-binding algorithm
FVVALIPLV

SEQ ID NO: 120 - protein, 9 aa, Homo sapiens
  PEPTIDE (1)..(9): Non-immunogenic peptide used for developing the TCR-binding algorithm
SLQNSEFLL

SEQ ID NO: 121 - protein, 9 aa, Homo sapiens
  PEPTIDE (1)..(9): Non-immunogenic peptide used for developing the TCR-binding algorithm
AYGSFVRTV

SEQ ID NO: 122 - protein, 9 aa, Homo sapiens
  PEPTIDE (1)..(9): Non-immunogenic peptide used for developing the TCR-binding algorithm
GLMTAVYLV

SEQ ID NO: 123 - protein, 9 aa, Homo sapiens
  PEPTIDE (1)..(9): Non-immunogenic peptide used for developing the TCR-binding algorithm
LMHAPAFET

SEQ ID NO: 124 - protein, 9 aa, Homo sapiens
  PEPTIDE (1)..(9): Non-immunogenic peptide used for developing the TCR-binding algorithm
GLYYLTTEV

SEQ ID NO: 125 - protein, 9 aa, Homo sapiens
  PEPTIDE (1)..(9): Non-immunogenic peptide used for developing the TCR-binding algorithm
LLYNEQFAV

SEQ ID NO: 126 - protein, 9 aa, Homo sapiens
  PEPTIDE (1)..(9): Non-immunogenic peptide used for developing the TCR-binding algorithm
VVFEDVKGT

SEQ ID NO: 127 - protein, 9 aa, Homo sapiens
  PEPTIDE (1)..(9): Non-immunogenic peptide used for developing the TCR-binding algorithm
ALSTGLIHL

SEQ ID NO: 128 - protein, 9 aa, Homo sapiens
  PEPTIDE (1)..(9): Non-immunogenic peptide used for developing the TCR-binding algorithm
FIPENQRTV

SEQ ID NO: 129 - protein, 9 aa, Homo sapiens
  PEPTIDE (1)..(9): Non-immunogenic peptide used for developing the TCR-binding algorithm
DLPSGFNTL

SEQ ID NO: 130 - protein, 9 aa, Homo sapiens
  PEPTIDE (1)..(9): Non-immunogenic peptide used for developing the TCR-binding algorithm
GLFGKGSLV

SEQ ID NO: 131 - protein, 9 aa, Homo sapiens
  PEPTIDE (1)..(9): Non-immunogenic peptide used for developing the TCR-binding algorithm
NSNDIVNAI

SEQ ID NO: 132 - protein, 9 aa, Homo sapiens
  PEPTIDE (1)..(9): Non-immunogenic peptide used for developing the TCR-binding algorithm
TVLRFVPPL

SEQ ID NO: 133 - protein, 9 aa, Homo sapiens
  PEPTIDE (1)..(9): Non-immunogenic peptide used for developing the TCR-binding algorithm
SLLEIGEGV

SEQ ID NO: 134 - protein, 9 aa, Homo sapiens
  PEPTIDE (1)..(9): Non-immunogenic peptide used for developing the TCR-binding algorithm
VIADYNYKL

SEQ ID NO: 135 - protein, 9 aa, Homo sapiens
  PEPTIDE (1)..(9): Non-immunogenic peptide used for developing the TCR-binding algorithm
AIMDKTVIL

SEQ ID NO: 136 - protein, 9 aa, Homo sapiens
  PEPTIDE (1)..(9): Non-immunogenic peptide used for developing the TCR-binding algorithm
KVLTLFAEV

SEQ ID NO: 137 - protein, 9 aa, Homo sapiens
  PEPTIDE (1)..(9): Non-immunogenic peptide used for developing the TCR-binding algorithm
SRAKAVRAL

SEQ ID NO: 138 - protein, 9 aa, Homo sapiens
  PEPTIDE (1)..(9): Non-immunogenic peptide used for developing the TCR-binding algorithm
KLDKEMEAV

SEQ ID NO: 139 - protein, 9 aa, Homo sapiens
  PEPTIDE (1)..(9): Non-immunogenic peptide used for developing the TCR-binding algorithm
VLADANETL

SEQ ID NO: 140 - protein, 9 aa, Homo sapiens
  PEPTIDE (1)..(9): Non-immunogenic peptide used for developing the TCR-binding algorithm
MLGNAPSVV

SEQ ID NO: 141 - protein, 9 aa, Homo sapiens
  PEPTIDE (1)..(9): Non-immunogenic peptide used for developing the TCR-binding algorithm
LLWQDPVPA

SEQ ID NO: 142 - protein, 9 aa, Homo sapiens
  PEPTIDE (1)..(9): Non-immunogenic peptide used for developing the TCR-binding algorithm
RLLEAFQFV

SEQ ID NO: 143 - protein, 9 aa, Homo sapiens
  PEPTIDE (1)..(9): Non-immunogenic peptide used for developing the TCR-binding algorithm
LLPPELSET

SEQ ID NO: 144 - protein, 9 aa, Homo sapiens
  PEPTIDE (1)..(9): Non-immunogenic peptide used for developing the TCR-binding algorithm
GLVDFVKHI

SEQ ID NO: 145 - protein, 9 aa, Homo sapiens
  PEPTIDE (1)..(9): Non-immunogenic peptide used for developing the TCR-binding algorithm
VLLEQMGSL

SEQ ID NO: 146 - protein, 9 aa, Homo sapiens
  PEPTIDE (1)..(9): Non-immunogenic peptide used for developing the TCR-binding algorithm
MLADKTKSI

SEQ ID NO: 147 - protein, 9 aa, Homo sapiens
  PEPTIDE (1)..(9): Non-immunogenic peptide used for developing the TCR-binding algorithm
LVLEQLGQL

SEQ ID NO: 148 - protein, 9 aa, Homo sapiens
  PEPTIDE (1)..(9): Non-immunogenic peptide used for developing the TCR-binding algorithm
RMPAVTDLV

SEQ ID NO: 149 - protein, 9 aa, Homo sapiens
  PEPTIDE (1)..(9): Non-immunogenic peptide used for developing the TCR-binding algorithm
TRVTIWKSK

SEQ ID NO: 150 - protein, 9 aa, Homo sapiens
  PEPTIDE (1)..(9): Non-immunogenic peptide used for developing the TCR-binding algorithm
YLSQIAVLL

SEQ ID NO: 151 - protein, 9 aa, Homo sapiens
  PEPTIDE (1)..(9): Non-immunogenic peptide used for developing the TCR-binding algorithm
YLLALRYLA

SEQ ID NO: 152 - protein, 9 aa, Homo sapiens
  PEPTIDE (1)..(9): Non-immunogenic peptide used for developing the TCR-binding algorithm
RLMIGTAAA

SEQ ID NO: 153 - protein, 9 aa, Homo sapiens
  PEPTIDE (1)..(9): Non-immunogenic peptide used for developing the TCR-binding algorithm
FLLPDAQSI

SEQ ID NO: 154 - protein, 9 aa, Homo sapiens
  PEPTIDE (1)..(9): Non-immunogenic peptide used for developing the TCR-binding algorithm
YTYKWETFL

SEQ ID NO: 155 - protein, 9 aa, Homo sapiens
  PEPTIDE (1)..(9): Non-immunogenic peptide used for developing the TCR-binding algorithm
AETGSGTAS

SEQ ID NO: 156 - protein, 9 aa, Homo sapiens
  PEPTIDE (1)..(9): Non-immunogenic peptide used for developing the TCR-binding algorithm
KLCTFSFLI

SEQ ID NO: 157 - protein, 9 aa, Homo sapiens
  PEPTIDE (1)..(9): Non-immunogenic peptide used for developing the TCR-binding algorithm
FLIHSADWL

SEQ ID NO: 158 - protein, 9 aa, Homo sapiens
  PEPTIDE (1)..(9): Non-immunogenic peptide used for developing the TCR-binding algorithm
ALWGPDPAA

SEQ ID NO: 159 - protein, 9 aa, Homo sapiens
  PEPTIDE (1)..(9): Non-immunogenic peptide used for developing the TCR-binding algorithm
NILFVITKL

SEQ ID NO: 160 - protein, 9 aa, Homo sapiens
  PEPTIDE (1)..(9): Non-immunogenic peptide used for developing the TCR-binding algorithm
LLACAVIHA

SEQ ID NO: 161 - protein, 9 aa, Homo sapiens
  PEPTIDE (1)..(9): Non-immunogenic peptide used for developing the TCR-binding algorithm
TLAARIKFL

SEQ ID NO: 162 - protein, 9 aa, Homo sapiens
  PEPTIDE (1)..(9): Non-immunogenic peptide used for developing the TCR-binding algorithm
DVVNGLANL

SEQ ID NO: 163 - protein, 9 aa, Homo sapiens
  PEPTIDE (1)..(9): Non-immunogenic peptide used for developing the TCR-binding algorithm
ALAPAPVEV

SEQ ID NO: 164 - protein, 9 aa, Homo sapiens
  PEPTIDE (1)..(9): Non-immunogenic peptide used for developing the TCR-binding algorithm
YLGKLFVTL

SEQ ID NO: 165 - protein, 9 aa, Homo sapiens
  PEPTIDE (1)..(9): Non-immunogenic peptide used for developing the TCR-binding algorithm
GADEDDIKA

SEQ ID NO: 166 - protein, 9 aa, Homo sapiens
  PEPTIDE (1)..(9): Non-immunogenic peptide used for developing the TCR-binding algorithm
KLLTKPWDV

SEQ ID NO: 167 - protein, 9 aa, Homo sapiens
  PEPTIDE (1)..(9): Non-immunogenic peptide used for developing the TCR-binding algorithm
WMHHNMDLV

SEQ ID NO: 168 - protein, 9 aa, Homo sapiens
  PEPTIDE (1)..(9): Non-immunogenic peptide used for developing the TCR-binding algorithm
GLYLSQIAV

SEQ ID NO: 169 - protein, 9 aa, Homo sapiens
  PEPTIDE (1)..(9): Non-immunogenic peptide used for developing the TCR-binding algorithm
ILFTFLHLA

SEQ ID NO: 170 - protein, 9 aa, Homo sapiens
  PEPTIDE (1)..(9): Non-immunogenic peptide used for developing the TCR-binding algorithm
TEVGQDQYV

SEQ ID NO: 171 - protein, 9 aa, Homo sapiens
  PEPTIDE (1)..(9): Non-immunogenic peptide used for developing the TCR-binding algorithm
TRHPATATV

SEQ ID NO: 172 - protein, 9 aa, Homo sapiens
  PEPTIDE (1)..(9): Non-immunogenic peptide used for developing the TCR-binding algorithm
LLFLGVVFL

SEQ ID NO: 173 - protein, 9 aa, Homo sapiens
  PEPTIDE (1)..(9): Non-immunogenic peptide used for developing the TCR-binding algorithm
ILSSLGLPV

SEQ ID NO: 174 - protein, 9 aa, Homo sapiens
  PEPTIDE (1)..(9): Non-immunogenic peptide used for developing the TCR-binding algorithm
FANYNFTLL

SEQ ID NO: 175 - protein, 9 aa, Homo sapiens
  PEPTIDE (1)..(9): Non-immunogenic peptide used for developing the TCR-binding algorithm
ILLSIARVV

SEQ ID NO: 176 - protein, 9 aa, Homo sapiens
  PEPTIDE (1)..(9): Non-immunogenic peptide used for developing the TCR-binding algorithm
IVYEAADAI

SEQ ID NO: 177 - protein, 9 aa, Homo sapiens
  PEPTIDE (1)..(9): Non-immunogenic peptide used for developing the TCR-binding algorithm
KFRVQGEAV

SEQ ID NO: 178 - protein, 9 aa, Homo sapiens
  PEPTIDE (1)..(9): Non-immunogenic peptide used for developing the TCR-binding algorithm
RLLDDTPEV

SEQ ID NO: 179 - protein, 9 aa, Homo sapiens
  PEPTIDE (1)..(9): Non-immunogenic peptide used for developing the TCR-binding algorithm
KIFCISIFL

SEQ ID NO: 180 - protein, 9 aa, Homo sapiens
  PEPTIDE (1)..(9): Non-immunogenic peptide used for developing the TCR-binding algorithm
AMLQDMAIL

SEQ ID NO: 181 - protein, 9 aa, Homo sapiens
  PEPTIDE (1)..(9): Non-immunogenic peptide used for developing the TCR-binding algorithm
ALVLLMLPV

SEQ ID NO: 182 - protein, 9 aa, Homo sapiens
  PEPTIDE (1)..(9): Non-immunogenic peptide used for developing the TCR-binding algorithm
MIAAYTAAL

SEQ ID NO: 183 - protein, 9 aa, Homo sapiens
  PEPTIDE (1)..(9): Non-immunogenic peptide used for developing the TCR-binding algorithm
AALGLWLSV

SEQ ID NO: 184 - protein, 9 aa, Homo sapiens
  PEPTIDE (1)..(9): Non-immunogenic peptide used for developing the TCR-binding algorithm
VLCPYMPKV

SEQ ID NO: 185 - protein, 9 aa, Homo sapiens
  PEPTIDE (1)..(9): Non-immunogenic peptide used for developing the TCR-binding algorithm
ALIIIRSLL

SEQ ID NO: 186 - protein, 9 aa, Homo sapiens
  PEPTIDE (1)..(9): Non-immunogenic peptide used for developing the TCR-binding algorithm
VLLLDVTPL

SEQ ID NO: 187 - protein, 9 aa, Homo sapiens
  PEPTIDE (1)..(9): Non-immunogenic peptide used for developing the TCR-binding algorithm
AIYHPQQFV

SEQ ID NO: 188 - protein, 9 aa, Homo sapiens
  PEPTIDE (1)..(9): Non-immunogenic peptide used for developing the TCR-binding algorithm
AMKADIQHV

SEQ ID NO: 189 - protein, 9 aa, Homo sapiens
  PEPTIDE (1)..(9): Non-immunogenic peptide used for developing the TCR-binding algorithm
ALLSDWLPA

SEQ ID NO: 190 - protein, 9 aa, Homo sapiens
  PEPTIDE (1)..(9): Non-immunogenic peptide used for developing the TCR-binding algorithm
RMFAANLGV

SEQ ID NO: 191 - protein, 9 aa, Homo sapiens
  PEPTIDE (1)..(9): Non-immunogenic peptide used for developing the TCR-binding algorithm
MLQDMAILT

SEQ ID NO: 192 - protein, 9 aa, Homo sapiens
  PEPTIDE (1)..(9): Non-immunogenic peptide used for developing the TCR-binding algorithm
ALLWAAGVL

SEQ ID NO: 193 - protein, 9 aa, Homo sapiens
  PEPTIDE (1)..(9): Non-immunogenic peptide used for developing the TCR-binding algorithm
LLFRFMRPL

SEQ ID NO: 194 - protein, 9 aa, Homo sapiens
  PEPTIDE (1)..(9): Non-immunogenic peptide used for developing the TCR-binding algorithm
RLGAVILFV

SEQ ID NO: 195 - protein, 9 aa, Homo sapiens
  PEPTIDE (1)..(9): Non-immunogenic peptide used for developing the TCR-binding algorithm
DLSRDLDSV

SEQ ID NO: 196 - protein, 9 aa, Homo sapiens
  PEPTIDE (1)..(9): Non-immunogenic peptide used for developing the TCR-binding algorithm
GLYGAQYDV

SEQ ID NO: 197 - protein, 9 aa, Homo sapiens
  PEPTIDE (1)..(9): Non-immunogenic peptide used for developing the TCR-binding algorithm
FLAVGGVLL

SEQ ID NO: 198 - protein, 9 aa, Homo sapiens
  PEPTIDE (1)..(9): Non-immunogenic peptide used for developing the TCR-binding algorithm
MLASTLTDA

SEQ ID NO: 199 - protein, 9 aa, Homo sapiens
  PEPTIDE (1)..(9): Non-immunogenic peptide used for developing the TCR-binding algorithm
YLVTSINKL

SEQ ID NO: 200 - protein, 9 aa, Homo sapiens
  PEPTIDE (1)..(9): Non-immunogenic peptide used for developing the TCR-binding algorithm
SLPKHNVTI

SEQ ID NO: 201 - protein, 9 aa, Homo sapiens
  PEPTIDE (1)..(9): Non-immunogenic peptide used for developing the TCR-binding algorithm
RLARAIIEL

SEQ ID NO: 202 - protein, 9 aa, Homo sapiens
  PEPTIDE (1)..(9): Non-immunogenic peptide used for developing the TCR-binding algorithm
MALLRLPLV

SEQ ID NO: 203 - protein, 9 aa, Homo sapiens
  PEPTIDE (1)..(9): Non-immunogenic peptide used for developing the TCR-binding algorithm
YKSPASDAY

SEQ ID NO: 204 - protein, 9 aa, Homo sapiens
  PEPTIDE (1)..(9): Non-immunogenic peptide used for developing the TCR-binding algorithm
KLSSFFQSV

SEQ ID NO: 205 - protein, 9 aa, Homo sapiens
  PEPTIDE (1)..(9): Non-immunogenic peptide used for developing the TCR-binding algorithm
AIMDKKIIL

SEQ ID NO: 206 - protein, 9 aa, Homo sapiens
  PEPTIDE (1)..(9): Immunogenic peptide used for developing the TCR-binding algorithm
SLKDVLVSV

SEQ ID NO: 207 - protein, 9 aa, Homo sapiens
  PEPTIDE (1)..(9): Immunogenic peptide used for developing the TCR-binding algorithm
VAALFFFDI

SEQ ID NO: 208 - protein, 9 aa, Homo sapiens
  PEPTIDE (1)..(9): Immunogenic peptide used for developing the TCR-binding algorithm
SLWGGDVVL

SEQ ID NO: 209 - protein, 9 aa, Homo sapiens
  PEPTIDE (1)..(9): Immunogenic peptide used for developing the TCR-binding algorithm
AMDTISVFL

SEQ ID NO: 210 - protein, 9 aa, Homo sapiens
  PEPTIDE (1)..(9): Immunogenic peptide used for developing the TCR-binding algorithm
VLLLWITAA

SEQ ID NO: 211 - protein, 9 aa, Homo sapiens
  PEPTIDE (1)..(9): Immunogenic peptide used for developing the TCR-binding algorithm
MLWYTVYNI

SEQ ID NO: 212 - protein, 9 aa, Homo sapiens
  PEPTIDE (1)..(9): Immunogenic peptide used for developing the TCR-binding algorithm
RVPGVAPTL

SEQ ID NO: 213 - protein, 9 aa, Homo sapiens
  PEPTIDE (1)..(9): Immunogenic peptide used for developing the TCR-binding algorithm
WLDEVKQAL

SEQ ID NO: 214 - protein, 9 aa, Homo sapiens
  PEPTIDE (1)..(9): Immunogenic peptide used for developing the TCR-binding algorithm
ALNTPKDHI

SEQ ID NO: 215 - protein, 9 aa, Homo sapiens
  PEPTIDE (1)..(9): Immunogenic peptide used for developing the TCR-binding algorithm
AWLVAAAEI

SEQ ID NO: 216 - protein, 9 aa, Homo sapiens
  PEPTIDE (1)..(9): Immunogenic peptide used for developing the TCR-binding algorithm
AILHTPGCV

SEQ ID NO: 217 - protein, 9 aa, Homo sapiens
  PEPTIDE (1)..(9): Immunogenic peptide used for developing the TCR-binding algorithm
GLLDQVAAL

SEQ ID NO: 218 - protein, 9 aa, Homo sapiens
  PEPTIDE (1)..(9): Immunogenic peptide used for developing the TCR-binding algorithm
SQQAQLAAA

SEQ ID NO: 219 - protein, 9 aa, Homo sapiens
  PEPTIDE (1)..(9): Immunogenic peptide used for developing the TCR-binding algorithm
TLKDIVLDL

SEQ ID NO: 220 - protein, 9 aa, Homo sapiens
  PEPTIDE (1)..(9): Immunogenic peptide used for developing the TCR-binding algorithm
MLNIPSINV

SEQ ID NO: 221 - protein, 9 aa, Homo sapiens
  PEPTIDE (1)..(9): Immunogenic peptide used for developing the TCR-binding algorithm
AIMDKVIIL

SEQ ID NO: 222 - protein, 9 aa, Homo sapiens
  PEPTIDE (1)..(9): Immunogenic peptide used for developing the TCR-binding algorithm
GILGFVYTL

SEQ ID NO: 223 - protein, 9 aa, Homo sapiens
  PEPTIDE (1)..(9): Immunogenic peptide used for developing the TCR-binding algorithm
TLEEFSAKL

SEQ ID NO: 224 - protein, 9 aa, Homo sapiens
  PEPTIDE (1)..(9): Immunogenic peptide used for developing the TCR-binding algorithm
LLDAHIPQL

SEQ ID NO: 225 - protein, 9 aa, Homo sapiens
  PEPTIDE (1)..(9): Immunogenic peptide used for developing the TCR-binding algorithm
RTLDKVLEV

SEQ ID NO: 226 - protein, 9 aa, Homo sapiens
  PEPTIDE (1)..(9): Immunogenic peptide used for developing the TCR-binding algorithm
YLESFCEDV

SEQ ID NO: 227 - protein, 9 aa, Homo sapiens
  PEPTIDE (1)..(9): Immunogenic peptide used for developing the TCR-binding algorithm
RMTENIVEV

SEQ ID NO: 228 - protein, 9 aa, Homo sapiens
  PEPTIDE (1)..(9): Immunogenic peptide used for developing the TCR-binding algorithm
GILGVVFTL

SEQ ID NO: 229 - protein, 9 aa, Homo sapiens
  PEPTIDE (1)..(9): Immunogenic peptide used for developing the TCR-binding algorithm
RGTPMVITV

SEQ ID NO: 230 - protein, 9 aa, Homo sapiens
  PEPTIDE (1)..(9): Immunogenic peptide used for developing the TCR-binding algorithm
HLGNVKYLV

SEQ ID NO: 231 - protein, 9 aa, Homo sapiens
  PEPTIDE (1)..(9): Immunogenic peptide used for developing the TCR-binding algorithm
VVPEDYWGV

SEQ ID NO: 232 - protein, 9 aa, Homo sapiens
  PEPTIDE (1)..(9): Immunogenic peptide used for developing the TCR-binding algorithm
VLNDILSRL

SEQ ID NO: 233 - protein, 9 aa, Homo sapiens
  PEPTIDE (1)..(9): Immunogenic peptide used for developing the TCR-binding algorithm
RLPLVLPAV

SEQ ID NO: 234 - protein, 9 aa, Homo sapiens
  PEPTIDE (1)..(9): Immunogenic peptide used for developing the TCR-binding algorithm
VLNETTNWL

SEQ ID NO: 235 - protein, 9 aa, Homo sapiens
  PEPTIDE (1)..(9): Immunogenic peptide used for developing the TCR-binding algorithm
ALSEDLLSI

SEQ ID NO: 236 - protein, 9 aa, Homo sapiens
  PEPTIDE (1)..(9): Immunogenic peptide used for developing the TCR-binding algorithm
GILGFIFTL

SEQ ID NO: 237 - protein, 9 aa, Homo sapiens
  PEPTIDE (1)..(9): Immunogenic peptide used for developing the TCR-binding algorithm
IMVLSFLFL

SEQ ID NO: 238 - protein, 9 aa, Homo sapiens
  PEPTIDE (1)..(9): Immunogenic peptide used for developing the TCR-binding algorithm
TLAPQVEPL

SEQ ID NO: 239 - protein, 9 aa, Homo sapiens
  PEPTIDE (1)..(9): Immunogenic peptide used for developing the TCR-binding algorithm
FTWEGLYNV

SEQ ID NO: 240 - protein, 9 aa, Homo sapiens
  PEPTIDE (1)..(9): Immunogenic peptide used for developing the TCR-binding algorithm
AIMDKTIIL

SEQ ID NO: 241 - protein, 9 aa, Homo sapiens
  PEPTIDE (1)..(9): Immunogenic peptide used for developing the TCR-binding algorithm
GVLGFVFTL

SEQ ID NO: 242 - protein, 9 aa, Homo sapiens
  PEPTIDE (1)..(9): Immunogenic peptide used for developing the TCR-binding algorithm
RLQGISPKI

SEQ ID NO: 243 - protein, 9 aa, Homo sapiens
  PEPTIDE (1)..(9): Immunogenic peptide used for developing the TCR-binding algorithm
ALLKDTVYT

SEQ ID NO: 244 - protein, 9 aa, Homo sapiens
  PEPTIDE (1)..(9): Immunogenic peptide used for developing the TCR-binding algorithm
LLLIWFRPV

SEQ ID NO: 245 - protein, 9 aa, Homo sapiens
  PEPTIDE (1)..(9): Immunogenic peptide used for developing the TCR-binding algorithm
VAANIVLTV

SEQ ID NO: 246 - protein, 9 aa, Homo sapiens
  PEPTIDE (1)..(9): Immunogenic peptide used for developing the TCR-binding algorithm
AMLHWSLIL

SEQ ID NO: 247 - protein, 9 aa, Homo sapiens
  PEPTIDE (1)..(9): Immunogenic peptide used for developing the TCR-binding algorithm
ALAVLSVTL

SEQ ID NO: 248 - protein, 9 aa, Homo sapiens
  PEPTIDE (1)..(9): Immunogenic peptide used for developing the TCR-binding algorithm
SGDGLVATG

SEQ ID NO: 249 - protein, 9 aa, Homo sapiens
  PEPTIDE (1)..(9): Immunogenic peptide used for developing the TCR-binding algorithm
GLSISGNLL

SEQ ID NO: 250 - protein, 9 aa, Homo sapiens
  PEPTIDE (1)..(9): Immunogenic peptide used for developing the TCR-binding algorithm
YLLPAIVHI

SEQ ID NO: 251 - protein, 9 aa, Homo sapiens
  PEPTIDE (1)..(9): Immunogenic peptide used for developing the TCR-binding algorithm
WILGFVFTL

SEQ ID NO: 252 - protein, 9 aa, Homo sapiens
  PEPTIDE (1)..(9): Immunogenic peptide used for developing the TCR-binding algorithm
SLSAYIIRV

SEQ ID NO: 253 - protein, 9 aa, Homo sapiens
  PEPTIDE (1)..(9): Immunogenic peptide used for developing the TCR-binding algorithm
KLVCSPAPC

SEQ ID NO: 254 - protein, 9 aa, Homo sapiens
  PEPTIDE (1)..(9): Immunogenic peptide used for developing the TCR-binding algorithm
YINTALLNA

SEQ ID NO: 255 - protein, 9 aa, Homo sapiens
  PEPTIDE (1)..(9): Immunogenic peptide used for developing the TCR-binding algorithm
ILLARLFLY

SEQ ID NO: 256 - protein, 9 aa, Homo sapiens
  PEPTIDE (1)..(9): Immunogenic peptide used for developing the TCR-binding algorithm
YLDKVRATV

SEQ ID NO: 257 - protein, 9 aa, Homo sapiens
  PEPTIDE (1)..(9): Immunogenic peptide used for developing the TCR-binding algorithm
FANNKFTLV

SEQ ID NO: 258 - protein, 9 aa, Homo sapiens
  PEPTIDE (1)..(9): Immunogenic peptide used for developing the TCR-binding algorithm
LLHTDFEQV

SEQ ID NO: 259 - protein, 9 aa, Homo sapiens
  PEPTIDE (1)..(9): Immunogenic peptide used for developing the TCR-binding algorithm
FLYELIWNV

SEQ ID NO: 260 - protein, 9 aa, Homo sapiens
  PEPTIDE (1)..(9): Immunogenic peptide used for developing the TCR-binding algorithm
LLCGNLLIL

SEQ ID NO: 261 - protein, 9 aa, Homo sapiens
  PEPTIDE (1)..(9): Immunogenic peptide used for developing the TCR-binding algorithm
LIDQYLYYL

SEQ ID NO: 262 - protein, 9 aa, Homo sapiens
  PEPTIDE (1)..(9): Immunogenic peptide used for developing the TCR-binding algorithm
LLYNCCYHV

SEQ ID NO: 263 - protein, 9 aa, Homo sapiens
  PEPTIDE (1)..(9): Immunogenic peptide used for developing the TCR-binding algorithm
RDVPMLITT

SEQ ID NO: 264 - protein, 9 aa, Homo sapiens
  PEPTIDE (1)..(9): Immunogenic peptide used for developing the TCR-binding algorithm
PESSQRPPL

SEQ ID NO: 265 - protein, 9 aa, Homo sapiens
  PEPTIDE (1)..(9): Immunogenic peptide used for developing the TCR-binding algorithm
LMGDKSENV

SEQ ID NO: 266 - protein, 9 aa, Homo sapiens
  PEPTIDE (1)..(9): Immunogenic peptide used for developing the TCR-binding algorithm
RLNELLAYV

SEQ ID NO: 267 - protein, 9 aa, Homo sapiens
  PEPTIDE (1)..(9): Immunogenic peptide used for developing the TCR-binding algorithm
RLWHYPCTI

SEQ ID NO: 268 - protein, 9 aa, Homo sapiens
  PEPTIDE (1)..(9): Immunogenic peptide used for developing the TCR-binding algorithm
LLMWEAVTV

SEQ ID NO: 269 - protein, 9 aa, Homo sapiens
  PEPTIDE (1)..(9): Immunogenic peptide used for developing the TCR-binding algorithm
GMLGFVFTL

SEQ ID NO: 270 - protein, 9 aa, Homo sapiens
  PEPTIDE (1)..(9): Immunogenic peptide used for developing the TCR-binding algorithm
LGYGFVNYI

SEQ ID NO: 271 - protein, 9 aa, Homo sapiens
  PEPTIDE (1)..(9): Immunogenic peptide used for developing the TCR-binding algorithm
LIVDAVLQL

SEQ ID NO: 272 - protein, 9 aa, Homo sapiens
  PEPTIDE (1)..(9): Immunogenic peptide used for developing the TCR-binding algorithm
FLLDILGAT

SEQ ID NO: 273 - protein, 9 aa, Homo sapiens
  PEPTIDE (1)..(9): Immunogenic peptide used for developing the TCR-binding algorithm
RLLQTGIHV

SEQ ID NO: 274 - protein, 9 aa, Homo sapiens
  PEPTIDE (1)..(9): Immunogenic peptide used for developing the TCR-binding algorithm
FLGERVTLT

SEQ ID NO: 275 - protein, 9 aa, Homo sapiens
  PEPTIDE (1)..(9): Immunogenic peptide used for developing the TCR-binding algorithm
FVNYDFTIV

SEQ ID NO: 276 - protein, 9 aa, Homo sapiens
  PEPTIDE (1)..(9): Immunogenic peptide used for developing the TCR-binding algorithm
KLNDWDFVV

SEQ ID NO: 277 - protein, 9 aa, Homo sapiens
  PEPTIDE (1)..(9): Immunogenic peptide used for developing the TCR-binding algorithm
LFLNTLSFV

SEQ ID NO: 278 - protein, 9 aa, Homo sapiens
  PEPTIDE (1)..(9): Immunogenic peptide used for developing the TCR-binding algorithm
GGNGMLATI

SEQ ID NO: 279 - protein, 9 aa, Homo sapiens
  PEPTIDE (1)..(9): Immunogenic peptide used for developing the TCR-binding algorithm
SFHSLHLLF

SEQ ID NO: 280 - protein, 9 aa, Homo sapiens
  PEPTIDE (1)..(9): Immunogenic peptide used for developing the TCR-binding algorithm
AIIIAVLLV

SEQ ID NO: 281 - protein, 9 aa, Homo sapiens
  PEPTIDE (1)..(9): Immunogenic peptide used for developing the TCR-binding algorithm
RFIAQLLLL

SEQ ID NO: 282 - protein, 9 aa, Homo sapiens
  PEPTIDE (1)..(9): Immunogenic peptide used for developing the TCR-binding algorithm
SIYVYALPL

SEQ ID NO: 283 - protein, 9 aa, Homo sapiens
  PEPTIDE (1)..(9): Immunogenic peptide used for developing the TCR-binding algorithm
PTLDKVLEV

SEQ ID NO: 284 - protein, 9 aa, Homo sapiens
  PEPTIDE (1)..(9): Immunogenic peptide used for developing the TCR-binding algorithm
LVLILYLCV

SEQ ID NO: 285 - protein, 9 aa, Homo sapiens
  PEPTIDE (1)..(9): Immunogenic peptide used for developing the TCR-binding algorithm
GMSRIGMEV

SEQ ID NO: 286 - protein, 9 aa, Homo sapiens
  PEPTIDE (1)..(9): Immunogenic peptide used for developing the TCR-binding algorithm
FLSHDFTLV

SEQ ID NO: 287 - protein, 9 aa, Homo sapiens
  PEPTIDE (1)..(9): Immunogenic peptide used for developing the TCR-binding algorithm
CINGVCWSV

SEQ ID NO: 288 - protein, 9 aa, Homo sapiens
  PEPTIDE (1)..(9): Immunogenic peptide used for developing the TCR-binding algorithm
SITEVECFL

SEQ ID NO: 289 - protein, 9 aa, Homo sapiens
  PEPTIDE (1)..(9): Immunogenic peptide used for developing the TCR-binding algorithm
RLERKWLDV

SEQ ID NO: 290 - protein, 9 aa, Homo sapiens
  PEPTIDE (1)..(9): Immunogenic peptide used for developing the TCR-binding algorithm
SIDQLCKTF

SEQ ID NO: 291 - protein, 9 aa, Homo sapiens
  PEPTIDE (1)..(9): Immunogenic peptide used for developing the TCR-binding algorithm
QLFNHTMFI

SEQ ID NO: 292 - protein, 9 aa, Homo sapiens
  PEPTIDE (1)..(9): Immunogenic peptide used for developing the TCR-binding algorithm
MIMQGGFSV

SEQ ID NO: 293 - protein, 9 aa, Homo sapiens
  PEPTIDE (1)..(9): Immunogenic peptide used for developing the TCR-binding algorithm
KCIDFYSRI

SEQ ID NO: 294 - protein, 9 aa, Homo sapiens
  PEPTIDE (1)..(9): Immunogenic peptide used for developing the TCR-binding algorithm
SLKKNSRSL

SEQ ID NO: 295 - protein, 9 aa, Homo sapiens
  PEPTIDE (1)..(9): Immunogenic peptide used for developing the TCR-binding algorithm
MLDLQPETT

SEQ ID NO: 296 - protein, 9 aa, Homo sapiens
  PEPTIDE (1)..(9): Immunogenic peptide used for developing the TCR-binding algorithm
MTIIFLILM

SEQ ID NO: 297 - protein, 9 aa, Homo sapiens
  PEPTIDE (1)..(9): Immunogenic peptide used for developing the TCR-binding algorithm
NDFCCVATV

SEQ ID NO: 298 - protein, 9 aa, Homo sapiens
  PEPTIDE (1)..(9): Immunogenic peptide used for developing the TCR-binding algorithm
YLEPGPVTA

SEQ ID NO: 299 - protein, 9 aa, Homo sapiens
  PEPTIDE (1)..(9): Immunogenic peptide used for developing the TCR-binding algorithm
ILDKKVEKV

SEQ ID NO: 300 - protein, 9 aa, Homo sapiens
  PEPTIDE (1)..(9): Immunogenic peptide used for developing the TCR-binding algorithm
LALLLLDRL

SEQ ID NO: 301 - protein, 9 aa, Homo sapiens
  PEPTIDE (1)..(9): Immunogenic peptide used for developing the TCR-binding algorithm
FIDKFTPPV

SEQ ID NO: 302 - protein, 9 aa, Homo sapiens
  PEPTIDE (1)..(9): Immunogenic peptide used for developing the TCR-binding algorithm
GILEFVFTL

SEQ ID NO: 303 - protein, 9 aa, Homo sapiens
  PEPTIDE (1)..(9): Immunogenic peptide used for developing the TCR-binding algorithm
YLVSIFLHL

SEQ ID NO: 304 - protein, 9 aa, Homo sapiens
  PEPTIDE (1)..(9): Immunogenic peptide used for developing the TCR-binding algorithm
FVVPILLKA

SEQ ID NO: 305 - protein, 9 aa, Homo sapiens
  PEPTIDE (1)..(9): Immunogenic peptide used for developing the TCR-binding algorithm
CLPACVYGL

SEQ ID NO: 306 - protein, 9 aa, Homo sapiens
  PEPTIDE (1)..(9): Immunogenic peptide used for developing the TCR-binding algorithm
VLSEWLPVT

SEQ ID NO: 307 - protein, 9 aa, Homo sapiens
  PEPTIDE (1)..(9): Immunogenic peptide used for developing the TCR-binding algorithm
TLLDHIRTA

SEQ ID NO: 308 - protein, 9 aa, Homo sapiens
  PEPTIDE (1)..(9): Immunogenic peptide used for developing the TCR-binding algorithm
KMLKEMGEV

SEQ ID NO: 309 - protein, 9 aa, Homo sapiens
  PEPTIDE (1)..(9): Immunogenic peptide used for developing the TCR-binding algorithm
AVADHVAAV

SEQ ID NO: 310 - protein, 9 aa, Homo sapiens
  PEPTIDE (1)..(9): Immunogenic peptide used for developing the TCR-binding algorithm
TLNDLETDV

SEQ ID NO: 311 - protein, 9 aa, Homo sapiens
  PEPTIDE (1)..(9): Immunogenic peptide used for developing the TCR-binding algorithm
TLLANVTAV

SEQ ID NO: 312 - protein, 9 aa, Homo sapiens
  PEPTIDE (1)..(9): Immunogenic peptide used for developing the TCR-binding algorithm
SLVNGVVRL

SEQ ID NO: 313 - protein, 9 aa, Homo sapiens
  PEPTIDE (1)..(9): Immunogenic peptide used for developing the TCR-binding algorithm
ALPHIIDEV

SEQ ID NO: 314 - protein, 9 aa, Homo sapiens
  PEPTIDE (1)..(9): Immunogenic peptide used for developing the TCR-binding algorithm
LITGRLQSL

SEQ ID NO: 315 - protein, 9 aa, Homo sapiens
  PEPTIDE (1)..(9): Immunogenic peptide used for developing the TCR-binding algorithm
TLTSYWRRV

SEQ ID NO: 316 - protein, 9 aa, Homo sapiens
  PEPTIDE (1)..(9): Immunogenic peptide used for developing the TCR-binding algorithm
FQGRGVFEL

SEQ ID NO: 317 - protein, 9 aa, Homo sapiens
  PEPTIDE (1)..(9): Immunogenic peptide used for developing the TCR-binding algorithm
SLMDLLSSL

SEQ ID NO: 318 - protein, 9 aa, Homo sapiens
  PEPTIDE (1)..(9): Immunogenic peptide used for developing the TCR-binding algorithm
FLTSVINRV

SEQ ID NO: 319 - protein, 9 aa, Homo sapiens
  PEPTIDE (1)..(9): Immunogenic peptide used for developing the TCR-binding algorithm
GILDFGVKL

SEQ ID NO: 320 - protein, 9 aa, Homo sapiens
  PEPTIDE (1)..(9): Immunogenic peptide used for developing the TCR-binding algorithm
QLVQSGAEV

SEQ ID NO: 321 - protein, 9 aa, Homo sapiens
  PEPTIDE (1)..(9): Immunogenic peptide used for developing the TCR-binding algorithm
YLLKPVQRI

SEQ ID NO: 322 - protein, 9 aa, Homo sapiens
  PEPTIDE (1)..(9): Immunogenic peptide used for developing the TCR-binding algorithm
SLPITVYYA

SEQ ID NO: 323 - protein, 9 aa, Homo sapiens
  PEPTIDE (1)..(9): Immunogenic peptide used for developing the TCR-binding algorithm
AIMDKNITL

SEQ ID NO: 324 - protein, 9 aa, Homo sapiens
  PEPTIDE (1)..(9): Immunogenic peptide used for developing the TCR-binding algorithm
RINAILATA

SEQ ID NO: 325 - protein, 9 aa, Homo sapiens
  PEPTIDE (1)..(9): Immunogenic peptide used for developing the TCR-binding algorithm
FVNHRFTLV

SEQ ID NO: 326 - protein, 9 aa, Homo sapiens
  PEPTIDE (1)..(9): Immunogenic peptide used for developing the TCR-binding algorithm
LLSLFSLWL

SEQ ID NO: 327 - protein, 9 aa, Homo sapiens
  PEPTIDE (1)..(9): Immunogenic peptide used for developing the TCR-binding algorithm
ALAEGDLLA

SEQ ID NO: 328 - protein, 9 aa, Homo sapiens
  PEPTIDE (1)..(9): Immunogenic peptide used for developing the TCR-binding algorithm
TLARGFPFV

SEQ ID NO: 329 - protein, 9 aa, Homo sapiens
  PEPTIDE (1)..(9): Immunogenic peptide used for developing the TCR-binding algorithm
LIFLARSAL

SEQ ID NO: 330 - protein, 9 aa, Homo sapiens
  PEPTIDE (1)..(9): Immunogenic peptide used for developing the TCR-binding algorithm
ILLWEIPDV

SEQ ID NO: 331 - protein, 9 aa, Homo sapiens
  PEPTIDE (1)..(9): Immunogenic peptide used for developing the TCR-binding algorithm
HLMIDRPYV

SEQ ID NO: 332 - protein, 9 aa, Homo sapiens
  PEPTIDE (1)..(9): Immunogenic peptide used for developing the TCR-binding algorithm
ALISAFSGS

SEQ ID NO: 333 - protein, 9 aa, Homo sapiens
  PEPTIDE (1)..(9): Immunogenic peptide used for developing the TCR-binding algorithm
RQYDPVAAL

SEQ ID NO: 334 - protein, 9 aa, Homo sapiens
  PEPTIDE (1)..(9): Immunogenic peptide used for developing the TCR-binding algorithm
LLIGGFAGL

SEQ ID NO: 335 - protein, 9 aa, Homo sapiens
  PEPTIDE (1)..(9): Immunogenic peptide used for developing the TCR-binding algorithm
FANYKFTLV

SEQ ID NO: 336 - protein, 9 aa, Homo sapiens
  PEPTIDE (1)..(9): Immunogenic peptide used for developing the TCR-binding algorithm
VPILLKALY

SEQ ID NO: 337 - protein, 9 aa, Homo sapiens
  PEPTIDE (1)..(9): Immunogenic peptide used for developing the TCR-binding algorithm
LLWNGPMAV

SEQ ID NO: 338 - protein, 9 aa, Homo sapiens
  PEPTIDE (1)..(9): Immunogenic peptide used for developing the TCR-binding algorithm
KLSDYEGRL

SEQ ID NO: 339 - protein, 9 aa, Homo sapiens
  PEPTIDE (1)..(9): Immunogenic peptide used for developing the TCR-binding algorithm
GMVTTSTTL

SEQ ID NO: 340 - protein, 9 aa, Homo sapiens
  PEPTIDE (1)..(9): Immunogenic peptide used for developing the TCR-binding algorithm
SLVEELKKV

SEQ ID NO: 341 - protein, 9 aa, Homo sapiens
  PEPTIDE (1)..(9): Immunogenic peptide used for developing the TCR-binding algorithm
ALSALLTKL

SEQ ID NO: 342 - protein, 9 aa, Homo sapiens
  PEPTIDE (1)..(9): Immunogenic peptide used for developing the TCR-binding algorithm
FVDYNFSLV

SEQ ID NO: 343 - protein, 9 aa, Homo sapiens
  PEPTIDE (1)..(9): Immunogenic peptide used for developing the TCR-binding algorithm
ILLNKHIDA

SEQ ID NO: 344 - protein, 9 aa, Homo sapiens
  PEPTIDE (1)..(9): Immunogenic peptide used for developing the TCR-binding algorithm
ILNNPKASL

SEQ ID NO: 345 - protein, 9 aa, Homo sapiens
  PEPTIDE (1)..(9): Immunogenic peptide used for developing the TCR-binding algorithm
FQQLFLNTL

SEQ ID NO: 346 - protein, 9 aa, Homo sapiens
  PEPTIDE (1)..(9): Immunogenic peptide used for developing the TCR-binding algorithm
ALLGLTLGV

SEQ ID NO: 347 - protein, 9 aa, Homo sapiens
  PEPTIDE (1)..(9): Immunogenic peptide used for developing the TCR-binding algorithm
GLMWLSYFV

SEQ ID NO: 348 - protein, 9 aa, Homo sapiens
  PEPTIDE (1)..(9): Immunogenic peptide used for developing the TCR-binding algorithm
KVDDTFYYV

SEQ ID NO: 349 - protein, 9 aa, Homo sapiens
  PEPTIDE (1)..(9): Immunogenic peptide used for developing the TCR-binding algorithm
ALFHEVAKL

SEQ ID NO: 350 - protein, 9 aa, Homo sapiens
  PEPTIDE (1)..(9): Immunogenic peptide used for developing the TCR-binding algorithm
SLPRSRTPI

SEQ ID NO: 351 - protein, 9 aa, Homo sapiens
  PEPTIDE (1)..(9): Immunogenic peptide used for developing the TCR-binding algorithm
LMLIWYRPV

SEQ ID NO: 352 - protein, 9 aa, Homo sapiens
  PEPTIDE (1)..(9): Immunogenic peptide used for developing the TCR-binding algorithm
IVIEAIHTV

SEQ ID NO: 353 - protein, 9 aa, Homo sapiens
  PEPTIDE (1)..(9): Immunogenic peptide used for developing the TCR-binding algorithm
SLILVSQYT

SEQ ID NO: 354 - protein, 9 aa, Homo sapiens
  PEPTIDE (1)..(9): Immunogenic peptide used for developing the TCR-binding algorithm
GTLGFVFTL

SEQ ID NO: 355 - protein, 9 aa, Homo sapiens
  PEPTIDE (1)..(9): Immunogenic peptide used for developing the TCR-binding algorithm
RLNEVAKNL

SEQ ID NO: 356 - protein, 9 aa, Homo sapiens
  PEPTIDE (1)..(9): Immunogenic peptide used for developing the TCR-binding algorithm
MINAYLDKL

SEQ ID NO: 357 - protein, 9 aa, Homo sapiens
  PEPTIDE (1)..(9): Immunogenic peptide used for developing the TCR-binding algorithm
TIDQLCKTF

SEQ ID NO: 358 - protein, 9 aa, Homo sapiens
  PEPTIDE (1)..(9): Immunogenic peptide used for developing the TCR-binding algorithm
LVLPILITI

SEQ ID NO: 359 - protein, 9 aa, Homo sapiens
  PEPTIDE (1)..(9): Immunogenic peptide used for developing the TCR-binding algorithm
AIVDKNITL

SEQ ID NO: 360 - protein, 9 aa, Homo sapiens
  PEPTIDE (1)..(9): Immunogenic peptide used for developing the TCR-binding algorithm
RLIQNSITI

SEQ ID NO: 361 - protein, 9 aa, Homo sapiens
  PEPTIDE (1)..(9): Immunogenic peptide used for developing the TCR-binding algorithm
ILRSFIPLL

SEQ ID NO: 362 - protein, 9 aa, Homo sapiens
  PEPTIDE (1)..(9): Immunogenic peptide used for developing the TCR-binding algorithm
FANHNFTLV

SEQ ID NO: 363 - protein, 9 aa, Homo sapiens
  PEPTIDE (1)..(9): Immunogenic peptide used for developing the TCR-binding algorithm
QLSTRGVQI

SEQ ID NO: 364 - protein, 9 aa, Homo sapiens
  PEPTIDE (1)..(9): Immunogenic peptide used for developing the TCR-binding algorithm
LLSILCIWV

SEQ ID NO: 365 - protein, 9 aa, Homo sapiens
  PEPTIDE (1)..(9): Immunogenic peptide used for developing the TCR-binding algorithm
PTLDKVLEL

SEQ ID NO: 366 - protein, 9 aa, Homo sapiens
  PEPTIDE (1)..(9): Immunogenic peptide used for developing the TCR-binding algorithm
GVRVLEDGV

SEQ ID NO: 367 - protein, 9 aa, Homo sapiens
  PEPTIDE (1)..(9): Immunogenic peptide used for developing the TCR-binding algorithm
MVMELIRMI

SEQ ID NO: 368 - protein, 9 aa, Homo sapiens
  PEPTIDE (1)..(9): Immunogenic peptide used for developing the TCR-binding algorithm
SAPLPSNRV

SEQ ID NO: 369 - protein, 9 aa, Homo sapiens
  PEPTIDE (1)..(9): Immunogenic peptide used for developing the TCR-binding algorithm
LQLCCLATA

SEQ ID NO: 370 - protein, 9 aa, Homo sapiens
  PEPTIDE (1)..(9): Immunogenic peptide used for developing the TCR-binding algorithm
ELTEVFEFA

SEQ ID NO: 371 - protein, 9 aa, Homo sapiens
  PEPTIDE (1)..(9): Immunogenic peptide used for developing the TCR-binding algorithm
CLTEYILWV

SEQ ID NO: 372 - protein, 9 aa, Homo sapiens
  PEPTIDE (1)..(9): Immunogenic peptide used for developing the TCR-binding algorithm
YLIIGILTL

SEQ ID NO: 373 - protein, 9 aa, Homo sapiens
  PEPTIDE (1)..(9): Immunogenic peptide used for developing the TCR-binding algorithm
GILGLVFTL

SEQ ID NO: 374 - protein, 9 aa, Homo sapiens
  PEPTIDE (1)..(9): Immunogenic peptide used for developing the TCR-binding algorithm
AMLNGLIYV

SEQ ID NO: 375 - protein, 9 aa, Homo sapiens
  PEPTIDE (1)..(9): Immunogenic peptide used for developing the TCR-binding algorithm
RMLPHAPGV

SEQ ID NO: 376 - protein, 9 aa, Homo sapiens
  PEPTIDE (1)..(9): Immunogenic peptide used for developing the TCR-binding algorithm
LLIDLTSFL

SEQ ID NO: 377 - protein, 9 aa, Homo sapiens
  PEPTIDE (1)..(9): Immunogenic peptide used for developing the TCR-binding algorithm
LLLGTLNIV

SEQ ID NO: 378 - protein, 9 aa, Homo sapiens
  PEPTIDE (1)..(9): Immunogenic peptide used for developing the TCR-binding algorithm
FANYNFTLV

SEQ ID NO: 379 - protein, 9 aa, Homo sapiens
  PEPTIDE (1)..(9): Immunogenic peptide used for developing the TCR-binding algorithm
LGYGFVNYV

SEQ ID NO: 380 - protein, 9 aa, Homo sapiens
  PEPTIDE (1)..(9): Immunogenic peptide used for developing the TCR-binding algorithm
TLACFAVYT

SEQ ID NO: 381 - protein, 9 aa, Homo sapiens
  PEPTIDE (1)..(9): Immunogenic peptide used for developing the TCR-binding algorithm
SLNQTVHSL

SEQ ID NO: 382 - protein, 9 aa, Homo sapiens
  PEPTIDE (1)..(9): Immunogenic peptide used for developing the TCR-binding algorithm
RLNTVLATA

SEQ ID NO: 383 - protein, 9 aa, Homo sapiens
  PEPTIDE (1)..(9): Immunogenic peptide used for developing the TCR-binding algorithm
IVSPFIPLL

SEQ ID NO: 384 - protein, 9 aa, Homo sapiens
  PEPTIDE (1)..(9): Immunogenic peptide used for developing the TCR-binding algorithm
LLIEGIFFI

SEQ ID NO: 385 - protein, 9 aa, Homo sapiens
  PEPTIDE (1)..(9): Immunogenic peptide used for developing the TCR-binding algorithm
SIVAYTMSL

SEQ ID NO: 386 - protein, 9 aa, Homo sapiens
  PEPTIDE (1)..(9): Immunogenic peptide used for developing the TCR-binding algorithm
ELLRPTTLV

SEQ ID NO: 387 - protein, 9 aa, Homo sapiens
  PEPTIDE (1)..(9): Immunogenic peptide used for developing the TCR-binding algorithm
FAFKDLFVV

SEQ ID NO: 388 - protein, 9 aa, Homo sapiens
  PEPTIDE (1)..(9): Immunogenic peptide used for developing the TCR-binding algorithm
NIVCPLCTL

SEQ ID NO: 389 - protein, 9 aa, Homo sapiens
  PEPTIDE (1)..(9): Immunogenic peptide used for developing the TCR-binding algorithm
GGPNLDNIL

SEQ ID NO: 390 - protein, 9 aa, Homo sapiens
  PEPTIDE (1)..(9): Immunogenic peptide used for developing the TCR-binding algorithm
TIPEALAAV

SEQ ID NO: 391 - protein, 9 aa, Homo sapiens
  PEPTIDE (1)..(9): Immunogenic peptide used for developing the TCR-binding algorithm
TLLYVLFEV

SEQ ID NO: 392 - protein, 9 aa, Homo sapiens
  PEPTIDE (1)..(9): Immunogenic peptide used for developing the TCR-binding algorithm
FLYGALLLA

SEQ ID NO: 393 - protein, 9 aa, Homo sapiens
  PEPTIDE (1)..(9): Immunogenic peptide used for developing the TCR-binding algorithm
LLDVAPLSL

SEQ ID NO: 394 - protein, 9 aa, Homo sapiens
  PEPTIDE (1)..(9): Immunogenic peptide used for developing the TCR-binding algorithm
MGLPGVATV

SEQ ID NO: 395 - protein, 9 aa, Homo sapiens
  PEPTIDE (1)..(9): Immunogenic peptide used for developing the TCR-binding algorithm
FANCNFTLV

SEQ ID NO: 396 - protein, 9 aa, Homo sapiens
  PEPTIDE (1)..(9): Immunogenic peptide used for developing the TCR-binding algorithm
NLNESLIDL

SEQ ID NO: 397 - protein, 9 aa, Homo sapiens
  PEPTIDE (1)..(9): Immunogenic peptide used for developing the TCR-binding algorithm
LLWSYAMGV

SEQ ID NO: 398 - protein, 9 aa, Homo sapiens
  PEPTIDE (1)..(9): Immunogenic peptide used for developing the TCR-binding algorithm
FMVFLQTHI

SEQ ID NO: 399 - protein, 9 aa, Homo sapiens
  PEPTIDE (1)..(9): Immunogenic peptide used for developing the TCR-binding algorithm
RVNRLIIWV

SEQ ID NO: 400 - protein, 9 aa, Homo sapiens
  PEPTIDE (1)..(9): Immunogenic peptide used for developing the TCR-binding algorithm
SLMSGVEPL

SEQ ID NO: 401 - protein, 9 aa, Homo sapiens
  PEPTIDE (1)..(9): Immunogenic peptide used for developing the TCR-binding algorithm
TLDYKPLSV

SEQ ID NO: 402 - protein, 9 aa, Homo sapiens
  PEPTIDE (1)..(9): Immunogenic peptide used for developing the TCR-binding algorithm
SLFNTVATL

SEQ ID NO: 403 - protein, 9 aa, Homo sapiens
  PEPTIDE (1)..(9): Immunogenic peptide used for developing the TCR-binding algorithm
VLLRHSKNV

SEQ ID NO: 404 - protein, 9 aa, Homo sapiens
  PEPTIDE (1)..(9): Immunogenic peptide used for developing the TCR-binding algorithm
VLLCVCLLI

SEQ ID NO: 405 - protein, 9 aa, Homo sapiens
  PEPTIDE (1)..(9): Immunogenic peptide used for developing the TCR-binding algorithm
SLLMWITQC

SEQ ID NO: 406 - protein, 9 aa, Homo sapiens
  PEPTIDE (1)..(9): Immunogenic peptide used for developing the TCR-binding algorithm
GLNDYLHSV

SEQ ID NO: 407 - protein, 9 aa, Homo sapiens
  PEPTIDE (1)..(9): Immunogenic peptide used for developing the TCR-binding algorithm
AMASTEGNV

SEQ ID NO: 408 - protein, 9 aa, Homo sapiens
  PEPTIDE (1)..(9): Immunogenic peptide used for developing the TCR-binding algorithm
GLREDLLSL

SEQ ID NO: 409 - protein, 9 aa, Homo sapiens
  PEPTIDE (1)..(9): Immunogenic peptide used for developing the TCR-binding algorithm
KLWCRHFCV

SEQ ID NO: 410 - protein, 9 aa, Homo sapiens
  PEPTIDE (1)..(9): Immunogenic peptide used for developing the TCR-binding algorithm
ALAIIIAVL

SEQ ID NO: 411 - protein, 9 aa, Homo sapiens
  PEPTIDE (1)..(9): Immunogenic peptide used for developing the TCR-binding algorithm
LQLPQGTTL

SEQ ID NO: 412 - protein, 9 aa, Homo sapiens
  PEPTIDE (1)..(9): Immunogenic peptide used for developing the TCR-binding algorithm
FLWEDQTLL

SEQ ID NO: 413 - protein, 9 aa, Homo sapiens
  PEPTIDE (1)..(9): Immunogenic peptide used for developing the TCR-binding algorithm
FLLKLTPLL

SEQ ID NO: 414 - protein, 9 aa, Homo sapiens
  PEPTIDE (1)..(9): Immunogenic peptide used for developing the TCR-binding algorithm
GIWGFVFTL

SEQ ID NO: 415 - protein, 9 aa, Homo sapiens
  PEPTIDE (1)..(9): Immunogenic peptide used for developing the TCR-binding algorithm
FANHKFTLV

SEQ ID NO: 416 - protein, 9 aa, Homo sapiens
  PEPTIDE (1)..(9): Immunogenic peptide used for developing the TCR-binding algorithm
QMMRNEFRV

SEQ ID NO: 417 - protein, 9 aa, Homo sapiens
  PEPTIDE (1)..(9): Immunogenic peptide used for developing the TCR-binding algorithm
FLLCFCVLL

SEQ ID NO: 418 - protein, 9 aa, Homo sapiens
  PEPTIDE (1)..(9): Immunogenic peptide used for developing the TCR-binding algorithm
GILTVSVAV

SEQ ID NO: 419 - protein, 9 aa, Homo sapiens
  PEPTIDE (1)..(9): Immunogenic peptide used for developing the TCR-binding algorithm
FVDYNFTIV

SEQ ID NO: 420 - protein, 9 aa, Homo sapiens
  PEPTIDE (1)..(9): Immunogenic peptide used for developing the TCR-binding algorithm
ALYDVVSKL

SEQ ID NO: 421 - protein, 9 aa, Homo sapiens
  PEPTIDE (1)..(9): Immunogenic peptide used for developing the TCR-binding algorithm
LFAAFPSFA

SEQ ID NO: 422 - protein, 9 aa, Homo sapiens
  PEPTIDE (1)..(9): Immunogenic peptide used for developing the TCR-binding algorithm
LLGRNSFEV

SEQ ID NO: 423 - protein, 9 aa, Homo sapiens
  PEPTIDE (1)..(9): Immunogenic peptide used for developing the TCR-binding algorithm
MLLDKNIPI

SEQ ID NO: 424 - protein, 9 aa, Homo sapiens
  PEPTIDE (1)..(9): Immunogenic peptide used for developing the TCR-binding algorithm
MLWGYLQYV

SEQ ID NO: 425 - protein, 9 aa, Homo sapiens
  PEPTIDE (1)..(9): Immunogenic peptide used for developing the TCR-binding algorithm
NLLTTPKFT

SEQ ID NO: 426 - protein, 9 aa, Homo sapiens
  PEPTIDE (1)..(9): Immunogenic peptide used for developing the TCR-binding algorithm
TLYAVATTI

SEQ ID NO: 427 - protein, 9 aa, Homo sapiens
  PEPTIDE (1)..(9): Immunogenic peptide used for developing the TCR-binding algorithm
FLKQQYMNL

SEQ ID NO: 428 - protein, 9 aa, Homo sapiens
  PEPTIDE (1)..(9): Immunogenic peptide used for developing the TCR-binding algorithm
KDLVLLATI

SEQ ID NO: 429 - protein, 9 aa, Homo sapiens
  PEPTIDE (1)..(9): Immunogenic peptide used for developing the TCR-binding algorithm
LLVSEIDWL

SEQ ID NO: 430 - protein, 9 aa, Homo sapiens
  PEPTIDE (1)..(9): Immunogenic peptide used for developing the TCR-binding algorithm
KLNPMLAKA

SEQ ID NO: 431 - protein, 9 aa, Homo sapiens
  PEPTIDE (1)..(9): Immunogenic peptide used for developing the TCR-binding algorithm
VIFDFLHCI

SEQ ID NO: 432 - protein, 9 aa, Homo sapiens
  PEPTIDE (1)..(9): Immunogenic peptide used for developing the TCR-binding algorithm
FANNEFTLV

SEQ ID NO: 433 - protein, 9 aa, Homo sapiens
  PEPTIDE (1)..(9): Immunogenic peptide used for developing the TCR-binding algorithm
VLCLRPVGA

SEQ ID NO: 434 - protein, 9 aa, Homo sapiens
  PEPTIDE (1)..(9): Immunogenic peptide used for developing the TCR-binding algorithm
SLFLGILSV

SEQ ID NO: 435 - protein, 9 aa, Homo sapiens
  PEPTIDE (1)..(9): Immunogenic peptide used for developing the TCR-binding algorithm
ALAHGVRAL

SEQ ID NO: 436 - protein, 9 aa, Homo sapiens
  PEPTIDE (1)..(9): Immunogenic peptide used for developing the TCR-binding algorithm
ALLALTRAI

SEQ ID NO: 437 - protein, 9 aa, Homo sapiens
  PEPTIDE (1)..(9): Immunogenic peptide used for developing the TCR-binding algorithm
NLLIRCLRC

SEQ ID NO: 438 - protein, 9 aa, Homo sapiens
  PEPTIDE (1)..(9): Immunogenic peptide used for developing the TCR-binding algorithm
SMINGVVKL

SEQ ID NO: 439 - protein, 9 aa, Homo sapiens
  PEPTIDE (1)..(9): Immunogenic peptide used for developing the TCR-binding algorithm
DVSRPTAVV

SEQ ID NO: 440 - protein, 9 aa, Homo sapiens
  PEPTIDE (1)..(9): Immunogenic peptide used for developing the TCR-binding algorithm
ALNTLVKQL

SEQ ID NO: 441 - protein, 9 aa, Homo sapiens
  PEPTIDE (1)..(9): Immunogenic peptide used for developing the TCR-binding algorithm
FIAGLIAIV

SEQ ID NO: 442 - protein, 9 aa, Homo sapiens
  PEPTIDE (1)..(9): Immunogenic peptide used for developing the TCR-binding algorithm
KTWGQYWQV

SEQ ID NO: 443 - protein, 9 aa, Homo sapiens
  PEPTIDE (1)..(9): Immunogenic peptide used for developing the TCR-binding algorithm
RMSKGVFKV

SEQ ID NO: 444 - protein, 9 aa, Homo sapiens
  PEPTIDE (1)..(9): Immunogenic peptide used for developing the TCR-binding algorithm
ILYGPLTRI

SEQ ID NO: 445 - protein, 9 aa, Homo sapiens
  PEPTIDE (1)..(9): Immunogenic peptide used for developing the TCR-binding algorithm
HLSLRGLPV

SEQ ID NO: 446 - protein, 9 aa, Homo sapiens
  PEPTIDE (1)..(9): Immunogenic peptide used for developing the TCR-binding algorithm
SLFGGMSWI

SEQ ID NO: 447 - protein, 9 aa, Homo sapiens
  PEPTIDE (1)..(9): Immunogenic peptide used for developing the TCR-binding algorithm
LLLLDVAPL

SEQ ID NO: 448 - protein, 9 aa, Homo sapiens
  PEPTIDE (1)..(9): Immunogenic peptide used for developing the TCR-binding algorithm
FLMEDQTLL

SEQ ID NO: 449 - protein, 9 aa, Homo sapiens
  PEPTIDE (1)..(9): Immunogenic peptide used for developing the TCR-binding algorithm
AMDSNTLEL

SEQ ID NO: 450 - protein, 9 aa, Homo sapiens
  PEPTIDE (1)..(9): Immunogenic peptide used for developing the TCR-binding algorithm
ITNCLLSTA

SEQ ID NO: 451 - protein, 9 aa, Homo sapiens
  PEPTIDE (1)..(9): Immunogenic peptide used for developing the TCR-binding algorithm
ILIEGIFFA

SEQ ID NO: 452 - protein, 9 aa, Homo sapiens
  PEPTIDE (1)..(9): Immunogenic peptide used for developing the TCR-binding algorithm
DLMGYIPAV

SEQ ID NO: 453 - protein, 9 aa, Homo sapiens
  PEPTIDE (1)..(9): Immunogenic peptide used for developing the TCR-binding algorithm
AMLVLLAEI

SEQ ID NO: 454 - protein, 9 aa, Homo sapiens
  PEPTIDE (1)..(9): Immunogenic peptide used for developing the TCR-binding algorithm
MINPLVITT

SEQ ID NO: 455 - protein, 9 aa, Homo sapiens
  PEPTIDE (1)..(9): Immunogenic peptide used for developing the TCR-binding algorithm
FILPVLGAV

SEQ ID NO: 456 - protein, 10 aa, Homo sapiens
  PEPTIDE (1)..(9): Immunogenic peptide used for developing the TCR-binding algorithm
SFAFRDLCIV

SEQ ID NO: 457 - protein, 9 aa, Homo sapiens
  PEPTIDE (1)..(9): Immunogenic peptide used for developing the TCR-binding algorithm
RMFPNAPYL

SEQ ID NO: 458 - protein, 9 aa, Homo sapiens
  PEPTIDE (1)..(9): Immunogenic peptide used for developing the TCR-binding algorithm
KVLIRCYLC

SEQ ID NO: 459 - protein, 9 aa, Homo sapiens
  PEPTIDE (1)..(9): Immunogenic peptide used for developing the TCR-binding algorithm
KLIVTPAAL

SEQ ID NO: 460 - protein, 9 aa, Homo sapiens
  PEPTIDE (1)..(9): Immunogenic peptide used for developing the TCR-binding algorithm
VLQELNVTV

SEQ ID NO: 461 - protein, 9 aa, Homo sapiens
  PEPTIDE (1)..(9): Immunogenic peptide used for developing the TCR-binding algorithm
LLNYILKSV

SEQ ID NO: 462 - protein, 9 aa, Homo sapiens
  PEPTIDE (1)..(9)
MMFGFHHSV

SEQ ID NO: 463 - protein, 9 aa, Homo sapiens
  PEPTIDE (1)..(9): Immunogenic peptide used for developing the TCR-binding algorithm
IVLGLIATA

SEQ ID NO: 464 - protein, 9 aa, Homo sapiens
  PEPTIDE (1)..(9): Immunogenic peptide used for developing the TCR-binding algorithm
YLNKIQNSL

SEQ ID NO: 465 - protein, 9 aa, Homo sapiens
  PEPTIDE (1)..(9): Immunogenic peptide used for developing the TCR-binding algorithm
QLLSSSKYT

SEQ ID NO: 466 - protein, 9 aa, Homo sapiens
  PEPTIDE (1)..(9): Immunogenic peptide used for developing the TCR-binding algorithm
CLFKDWEEL

SEQ ID NO: 467 - protein, 9 aa, Homo sapiens
  PEPTIDE (1)..(9): Immunogenic peptide used for developing the TCR-binding algorithm
AIIDPLIYA

SEQ ID NO: 468 - protein, 9 aa, Homo sapiens
  PEPTIDE (1)..(9): Immunogenic peptide used for developing the TCR-binding algorithm
TLGIVCPIC

SEQ ID NO: 469 - protein, 9 aa, Homo sapiens
  PEPTIDE (1)..(9): Immunogenic peptide used for developing the TCR-binding algorithm
KYQEFFWDA

SEQ ID NO: 470 - protein, 9 aa, Homo sapiens
  PEPTIDE (1)..(9): Immunogenic peptide used for developing the TCR-binding algorithm
LALPMPATA

SEQ ID NO: 471 - protein, 9 aa, Homo sapiens
  PEPTIDE (1)..(9): Immunogenic peptide used for developing the TCR-binding algorithm
SLMSWSAIL

SEQ ID NO: 472 - protein, 9 aa, Homo sapiens
  PEPTIDE (1)..(9): Immunogenic peptide used for developing the TCR-binding algorithm
KVLGLWATV

SEQ ID NO: 473 - protein, 9 aa, Homo sapiens
  PEPTIDE (1)..(9): Immunogenic peptide used for developing the TCR-binding algorithm
ILPDPLKPT

SEQ ID NO: 474 - protein, 9 aa, Homo sapiens
  PEPTIDE (1)..(9): Immunogenic peptide used for developing the TCR-binding algorithm
FLSFASLFL

SEQ ID NO: 475 - protein, 9 aa, Homo sapiens
  PEPTIDE (1)..(9): Immunogenic peptide used for developing the TCR-binding algorithm
ILIEGVFFA

SEQ ID NO: 476 - protein, 9 aa, Homo sapiens
  PEPTIDE (1)..(9): Immunogenic peptide used for developing the TCR-binding algorithm
ALLEDPVGT

SEQ ID NO: 477 - protein, 9 aa, Homo sapiens
  PEPTIDE (1)..(9): Immunogenic peptide used for developing the TCR-binding algorithm
FVNYNFTLV

SEQ ID NO: 478 - protein, 9 aa, Homo sapiens
  PEPTIDE (1)..(9): Immunogenic peptide used for developing the TCR-binding algorithm
WQWEHIPPA

SEQ ID NO: 479 - protein, 9 aa, Homo sapiens
  PEPTIDE (1)..(9): Immunogenic peptide used for developing the TCR-binding algorithm
VMLFILAGL

SEQ ID NO: 480 - protein, 9 aa, Homo sapiens
  PEPTIDE (1)..(9): Immunogenic peptide used for developing the TCR-binding algorithm
MTYAAPLFV

SEQ ID NO: 481 - protein, 9 aa, Homo sapiens
  PEPTIDE (1)..(9): Immunogenic peptide used for developing the TCR-binding algorithm
YLKKIKNSL

SEQ ID NO: 482 - protein, 9 aa, Homo sapiens
  PEPTIDE (1)..(9): Immunogenic peptide used for developing the TCR-binding algorithm
AMAGASTSA

SEQ ID NO: 483 - protein, 9 aa, Homo sapiens
  PEPTIDE (1)..(9): Immunogenic peptide used for developing the TCR-binding algorithm
NMLSTVLGV

SEQ ID NO: 484 - protein, 9 aa, Homo sapiens
  PEPTIDE (1)..(9): Immunogenic peptide used for developing the TCR-binding algorithm
ILAKFLHWL

SEQ ID NO: 485 - protein, 9 aa, Homo sapiens
  PEPTIDE (1)..(9): Immunogenic peptide used for developing the TCR-binding algorithm
KLGPGEEQV

SEQ ID NO: 486 - protein, 9 aa, Homo sapiens
  PEPTIDE (1)..(9): Immunogenic peptide used for developing the TCR-binding algorithm
SVYDFFVWL

SEQ ID NO: 487 - protein, 9 aa, Homo sapiens
  PEPTIDE (1)..(9): Immunogenic peptide used for developing the TCR-binding algorithm
VLTSESMHV

SEQ ID NO: 488 - protein, 9 aa, Homo sapiens
  PEPTIDE (1)..(9): Immunogenic peptide used for developing the TCR-binding algorithm
SLSRFSWGA

SEQ ID NO: 489 - protein, 9 aa, Homo sapiens
  PEPTIDE (1)..(9): Immunogenic peptide used for developing the TCR-binding algorithm
RMLGDVMAV

SEQ ID NO: 490 - protein, 9 aa, Homo sapiens
  PEPTIDE (1)..(9): Immunogenic peptide used for developing the TCR-binding algorithm
YILEETSVM

SEQ ID NO: 491 - protein, 9 aa, Homo sapiens
  PEPTIDE (1)..(9): Immunogenic peptide used for developing the TCR-binding algorithm
ILDAHSLYL

SEQ ID NO: 492 - protein, 9 aa, Homo sapiens
  PEPTIDE (1)..(9): Immunogenic peptide used for developing the TCR-binding algorithm
GIFEDRAPV

SEQ ID NO: 493 - protein, 9 aa, Homo sapiens
  PEPTIDE (1)..(9): Immunogenic peptide used for developing the TCR-binding algorithm
TVCGGIMFL

SEQ ID NO: 494 - protein, 9 aa, Homo sapiens
  PEPTIDE (1)..(9): Immunogenic peptide used for developing the TCR-binding algorithm
GLCPHCINV

SEQ ID NO: 495 - protein, 9 aa, Homo sapiens
  PEPTIDE (1)..(9): Immunogenic peptide used for developing the TCR-binding algorithm
AFLGERVTL

SEQ ID NO: 496 - protein, 9 aa, Homo sapiens
  PEPTIDE (1)..(9): Immunogenic peptide used for developing the TCR-binding algorithm
NGVRVLATA

SEQ ID NO: 497 - protein, 9 aa, Homo sapiens
  PEPTIDE (1)..(9): Immunogenic peptide used for developing the TCR-binding algorithm
QLLNSVLTL

SEQ ID NO: 498 - protein, 9 aa, Homo sapiens
  PEPTIDE (1)..(9): Immunogenic peptide used for developing the TCR-binding algorithm
ILHTNMPNV

SEQ ID NO: 499 - protein, 9 aa, Homo sapiens
  PEPTIDE (1)..(9): Immunogenic peptide used for developing the TCR-binding algorithm
AITEVECFL

SEQ ID NO: 500 - protein, 9 aa, Homo sapiens
  PEPTIDE (1)..(9): Immunogenic peptide used for developing the TCR-binding algorithm
GMDPRMCSL

SEQ ID NO: 501 - protein, 9 aa, Homo sapiens
  PEPTIDE (1)..(9): Immunogenic peptide used for developing the TCR-binding algorithm
AILIRVRNA

SEQ ID NO: 502 - protein, 9 aa, Homo sapiens
  PEPTIDE (1)..(9): Immunogenic peptide used for developing the TCR-binding algorithm
KTVLELTEV

SEQ ID NO: 503 - protein, 9 aa, Homo sapiens
  PEPTIDE (1)..(9): Immunogenic peptide used for developing the TCR-binding algorithm
VLHKRTLGL

SEQ ID NO: 504 - protein, 9 aa, Homo sapiens
  PEPTIDE (1)..(9): Immunogenic peptide used for developing the TCR-binding algorithm
MGNGCLRIV

SEQ ID NO: 505 - protein, 9 aa, Homo sapiens
  PEPTIDE (1)..(9): Immunogenic peptide used for developing the TCR-binding algorithm
LVMAQLLRI

SEQ ID NO: 506 - protein, 9 aa, Homo sapiens
  PEPTIDE (1)..(9): Immunogenic peptide used for developing the TCR-binding algorithm
AMLDLLKSV

SEQ ID NO: 507 - protein, 9 aa, Homo sapiens
  PEPTIDE (1)..(9): Immunogenic peptide used for developing the TCR-binding algorithm
IADAALAAL

SEQ ID NO: 508 - protein, 9 aa, Homo sapiens
  PEPTIDE (1)..(9): Immunogenic peptide used for developing the TCR-binding algorithm
DLSLRRFMV

SEQ ID NO: 509 - protein, 9 aa, Homo sapiens
  PEPTIDE (1)..(9): Immunogenic peptide used for developing the TCR-binding algorithm
LQDIEITCV

SEQ ID NO: 510 - protein, 9 aa, Homo sapiens
  PEPTIDE (1)..(9): Immunogenic peptide used for developing the TCR-binding algorithm
KLQEQQSDL

SEQ ID NO: 511 - protein, 9 aa, Homo sapiens
  PEPTIDE (1)..(9): Immunogenic peptide used for developing the TCR-binding algorithm
FLTCTDRSV

SEQ ID NO: 512 - protein, 9 aa, Homo sapiens
  PEPTIDE (1)..(9): Immunogenic peptide used for developing the TCR-binding algorithm
SVGGVFTSV

SEQ ID NO: 513 - protein, 9 aa, Homo sapiens
  PEPTIDE (1)..(9): immunogenic peptides from the mutations in head and neck cancer
STRDPLSKI

SEQ ID NO: 514 - protein, 9 aa, Homo sapiens
  PEPTIDE (1)..(9): immunogenic peptides from the mutations in head and neck cancer
RLLIELIWK

SEQ ID NO: 515 - protein, 9 aa, Homo sapiens
  PEPTIDE (1)..(9): immunogenic peptides from the mutations in head and neck cancer
STVESLTSQ

SEQ ID NO: 516 - protein, 9 aa, Homo sapiens
  PEPTIDE (1)..(9): immunogenic peptides from the mutations in head and neck cancer
LLIELIWKR

SEQ ID NO: 517 - protein, 9 aa, Homo sapiens
  PEPTIDE (1)..(9): immunogenic peptides from the mutations in head and neck cancer
LIELIWKRE

SEQ ID NO: 518 - protein, 9 aa, Homo sapiens
  PEPTIDE (1)..(9r): immunogenic peptides from the mutations in head and neck cancer
KITEQEKDF

SEQ ID NO: 519 - protein, 9 aa, Homo sapiens
  PEPTIDE (1)..(9): immunogenic peptides from the mutations in head and neck cancer
LTSQRKEMK

(19) United States
(12) Patent Application Publication
(10) Pub. No.: US 2019/0237158 A1
(43) Pub. Date: Aug. 1, 2019

(54) METHODS TO ANALYZE GENETIC ALTERATIONS IN CANCER TO IDENTIFY THERAPEUTIC PEPTIDE VACCINES AND KITS THEREFORE

(71) Applicant: MedGenome, Inc., Foster City, CA (US)

(72) Inventors: Amitabha Chaudhuri, Redwood City, CA (US); Ravi Gupta, Bangalore (IN); Priyanka Shah, Uttar Pradesh (IN); Malini Manoharan, Bangalore (IN); Kiran V. Paul, Kiran (IN); Rohit Goupta, Bangalore (IN)

(21) Appl. No.: 16/329,720

(22) PCT Filed: Aug. 31, 2017
(86) PCT No.: PCT/US17/49773

§ 371 Date: Feb. 28, 2019

Related U.S. Application Data
(60) Provisional application No. 62/382,179, filed on Aug. 31, 2016.

(51) Int. Cl. G16B 20/30; A61K 39/00; G16B 20/20; C12Q 1/6886; G16B 30/10; G16B 40/10

(52) U.S. Cl. CPC G16B 20/30; A61K 39/0011; G16B 20/20; A61P 35/00; G16B 30/10; G16B 40/10; C12Q 1/6886

(57) ABSTRACT

The invention describes a method for identifying T-cell activating neo-epitopes from all genetically altered proteins. The mutated proteins contribute to neo-epitopes after they are proteolytically degraded within antigen presenting cells, such as dendritic cells and macrophages.

### CROSS REFERENCE TO RELATED APPLICATIONS

The application claims priority to U.S. Provisional Application No. 62/382,179 filed Aug. 31, 2016, which is incorporated herein by reference in its entirely for all purposes.

### FIELD OF THE DISCLOSURE

The present disclosure is directed to methods of identifying immunogenic mutant peptides having therapeutic utility as cancer vaccines

### BACKGROUND OF THE INVENTION

Genetic alterations are detected in all tumor cells. These alterations, occurring at the level of DNA, are transcribed and translated to generate altered proteins that in many instances drive cancer. These altered proteins can sometime contribute to immune recognition by T and B cells evoking activation of the immune response, which can lead to the elimination of tumor cells expressing the altered proteins [1-3].

Tumor cells, including malignant tumor cells or cancer cells, accumulate a large number of somatic mutations, from as low as ten, to as high as thousands depending on the cancer type. Only a subset of these mutations can evoke an immune response. Identifying such mutations can lead to the generation of therapeutic vaccines that can be given to patient as a polypeptide or as nucleic acids (both DNA and RNA) [4].

For a mutation to be recognized as foreign, the mutant amino acid should be present as part of a peptide that binds class I or class II major histocompatibility complex (MHC or alternatively known as human leukocyte antigen or HLA in human) molecules and be presented on the surface of antigen presenting cells (professional APCs). The MHC- or HLA-bound peptide interacts with the T-cell receptor (TCR) expressed on the surface of T cells. Productive binding with the TCR activates T-cells, which can kill tumor cells directly through its cytolytic activity (CD8+ cytotoxic T-cells) or perform helper function (CD4+ helper T-cells) to induce antibody production. In this context, the definition of an immunogenic peptide is restricted to peptides that can interact with CD8⁺or CD4⁺T cells. For the interaction to happen, the peptide must be presented on the surface of cells in complex with MHC or HLA class I or class II proteins. The MHC class I- or HLA class I-bound peptide interacts with CD8⁺T cells, and the MHC class II- or HLA class II-bound peptide interacts with CD4⁺T cells. Although MHC or HLA binding and surface presentation is required for T cell activation, but, the displayed peptide bound to MHC or HLA proteins on the surface of cell is necessary but not sufficient for T cell activation as TCR must also interact with the displayed peptide. Most peptides presented on the cell surface in complex with MHC or HLA fail to engage T cells and therefore are not immunogenic [5]. Immunogenicity require not only peptide-binding and display by MHC class I or class II proteins but also binding of the MHC class I or class II- displayed peptide by TCR of the CD8+ T-cell or CD4+ T-cell respectively [6]. While much is known about the rules governing peptide binding by MHC or HLA molecules, little is known about the rules governing peptide binding by TCR, other than that the rules governing peptide binding by TCR are different from peptide binding by MHC or HLA proteins.

Class I HLA proteins are encoded by HLA-A, HLA-B and HLA-C genes. These proteins bind peptides of 8-11 amino acids in length, with the preferred length being 9 amino acids long. The peptide binding groove of class I HLA is formed by two alpha helices supported by an anti-parallel beta sheet. The peptide-binding groove is deeper compared to class II HLA molecules and requires residues to be projected outside the binding groove to make interactions with the TCR [7].

Peptides bind to class I HLA molecules in a multistep process. The steps are as follows: 1) generation of protein fragments by immunoproteasomal or proteasomal processing as part of the natural turnover of proteins in cells [8]; 2) Entry of the protein fragment into the lumen of the endoplasmic reticulum by binding to peptide transporters (TAP) [9]; 3) Binding to the peptide-binding groove of the class I HLA molecules; 4) Transport through vesicles to the cell surface and 5) presentation on the surface of cells [10] [11].

In the case of endogenous proteins, such as altered proteins in tumor or cancer cells, these proteins being produced intracellularly by the cell do not require cellular uptake. As such, peptides derived by immunoproteasomal or proteasomal processing as part of the natural turnover of proteins in cells may be displayed by class I MHC or HLA molecules in all cell types in which the altered protein is expressed by the cell. In contrast, in the case of a peptide used in tumor or cancer vaccine, The peptide is exogenous to the cell and must be taken up by professional antigen-presenting cells in a process called cross-presentation in order to be displayed by class I MHC or HLA proteins [12-14]. The peptide used in tumor or cancer vaccine is longer than the peptide displayed by class I MHC or HLA proteins, as the peptide is taken up by the cell and undergo proteolysis to produce shorter peptide(s). Equal number of amino acids are added to the amino- and carboxy-termini, so as to extend the length of the final peptide displayed by class I MHC or HLA proteins. Typically, live to eighteen amino acids are added to each end of the 8-11 amino acid long peptide displayed on cell surface by class I MHC or HLA proteins, such that the peptide formulated in the tumor or cancer vaccine is approximately 18 to 47 amino acids in length. The upper limit of peptide length in tumor or cancer vaccine is less than or equal to 50 amino acids. The antigen-presenting cells capable of cross presentation are professional antigen-presenting cells and include dendritic cells (primarily), macrophages, and B lymphocytes.

The binding of MHC-peptide complex to the CD8⁺T cells, henceforth referred to as cytolytic or cytotoxic T cells (CTLs) activates a series of signaling pathways in CTLs resulting in their expansion to generate a population of effector CTLs. These CTLs will recognize tumor cells displaying the mutant peptide on their surface and kill them by apoptosis. Therefore, peptides derived from cancer mutations that are capable of mounting a CTL response can be used as cancer vaccines for treating cancer patients [15].

Two studies have demonstrated that immunogenic peptides can provide long term benefit to cancer patients when used as monotherapy [16. 17]. Therefore, accurate identification of immunogenic peptides from tumor-derived mutant protein can provide an avenue of treatment for cancer patients [18] [19]. However, the lack of efficient method for identifying bonafide immunogenic peptides have not only increased the cost of vaccination, but also increased the uncertainty of whether the vaccine will deliver the desired effect of inducing an anti-tumor response.

Next generation sequencing technology can catalogue all tumor mutations from a patient's tumor cells rapidly. However, identifying immunogenic peptides derived from such mutations is still a formidable challenge. The challenge comes from the fact that accurate methods of selecting immunogenic peptides from a pool of immunogenic and non-immunogenic peptides [20] [18]. Most screening platform uses HLA-binding prediction as a measure of immunogenicity [21]. The prediction can be further confirmed by actual detection of the peptide on the cell surface by mass spectrometry [5]. However, surface presentation of a peptide in complex with HLA is not an indication of immunogenicity. For a peptide to be immunogenic, the peptide presented on the surface of cells must engage T cell receptor. There is a need in the art for a high throughput methodology for prediction of immunogenic peptide for cancer therapy.

### SUMMARY OF THE DISCLOSURE

The practice matter of the invention disclosed in this application has employed, unless otherwise indicated, computational prediction algorithms organized in a step-wise workflow to identify tumor or cancer vaccines from tumor-derived proteins, which are expressed and mutated or altered only in cancer cells. The invention covers the identification of T-cell neo-epitopes from four classes of genetically altered proteins—i) proteins altered in amino acid sequence in which one or more amino acids are altered or mutated, which may be arranged in a sequence or distributed randomly across the length of the protein; ii) proteins produced from genes with internal insertion or deletion in the coding sequence; iii) proteins translated from fusion genes; and iv) proteins produced from splice variants.

Selection of immunogenic peptides comprises: a) selecting a set of cancer variants from mouse and human cancer cell lines and mouse and human cancer tissues where each variant in the genomic sequence correspond to both protein coding and protein non-coding sequences; b) variants of mouse cell lines and cancer tissues are identified by mouse whole exome and/or whole genome sequencing and variants from human cancer cell lines and human cancer tissues are identified by whole exome and/or whole genome sequencing; c) variants in mouse tissues and cell lines are identified by comparing with the reference sequence of mouse, and variants in human tissues and cell lines are identified by comparing with the reference sequence of human; d) variants are identified by comparing with the reference sequence, where the reference sequence is mouse reference sequence available in the public domain, or human reference sequence available in the public domain (e.g., current mouse reference sequence is (GRCm38/mm10) and current human reference sequence is (hg19)); e) variants from mouse tissues and cell lines include all genomic variants that alter the sequence of the RNA and the sequence of the protein translated from the RNA; f) variants from human tissues and cell lines include all genomic variants that alter the sequence of the proteins translated from the messenger RNA-protein variants; g) selecting the variants based on their expression in the mouse or human cell lines and tissues from the transcriptomic analysis; h) generating 8-11 amino acid peptides from the altered protein variants; and i) selecting a set of 8-11 amino acid immunogenic peptides from the previous step by predicting immunogenicity of the variant peptide comprising the altered amino acids encoded by the variant coding sequence; thereby selecting immunogenic peptides from altered or mutated proteins unique to cancer or tumor cells or tissues.

In some embodiments, according to any of the methods described above, the method further comprises selecting peptides that bind T cells by engaging with the T cell receptor (TCR) by obtaining peptides that carry features of TCR binding. Steps include one or more of: a) determining features associated with each of the amino acids in a 9-mer peptide; b) determining features that are unique or shared between amino acids that make up the composition of the 9-mer peptide; c) determining features that favor interactions between TCR and the HLA-bound peptide, comprising amino acid positions 3-8 of the 9-mer peptide; d) determining features that favor HLA binding comprising amino acid positions 1-2 and 9 of the 9-mer peptide; e) determining features that are different between the non-mutated and the mutated peptide; g) determining and/or applying features that select immunogenic peptides from a list of immunogenic and non-immunogenic peptides thereby identifying immunogenic peptides from altered proteins expressed in tumor or cancer cell lines and/or tissues.

According to any one of the methods described above immunogenic peptide is defined by a combination of one or more of the following parameters: i) peptide is derived from a gene which is mutated in the DNA from tumor or cancer cell but not in normal cell as determined by DNA sequencing; ii) the mutant gene is expressed in tumor or cancer and detected by transcriptome sequencing; iii) mutation changes one or more amino acids in the translated protein determined by in silico protein translation (conceptual translation of protein coding region or sequences) from the transcript encoding the mutant protein; iv) mutated or altered peptide derived from the mutant or altered protein binds TCR; v) affinity of mutated peptide to class I HLA or equivalent; vi) sensitivity of the peptide to processing by proteasomal and/or immunoproteasomal enzymes and vii) ability of the peptide to bind peptide transporter present on the endoplasmic reticulum. In some embodiments, predicting immunogenicity is further based on HLA-typing analysis.

The present application in another aspect also provides tumor-specific immunogenic peptides identified by any of the above methods or combination of methods from human tumor patients. In some embodiments, the composition comprises of two or more tumor specific immunogenic mutant peptides described herein. In some embodiments, the composition further comprises an adjuvant

The present application in another aspect also provides cancer-specific immunogenic peptides identified by any of the above methods or combination of methods from human cancer patients. In some embodiments, the composition comprises of two or more cancer specific immunogenic mutant peptides described herein. In some embodiments, the composition further comprises an adjuvant

The present application in yet another aspect provides a method of creating an immunogenic composition comprising at least one tumor or cancer specific mutant peptide or a larger precursor encoding the 8- to 11-mer mutant immunogenic peptide identified by any of the methods described herein. In one embodiment, the method of creating an immunogenic composition comprises at least one tumor specific mutant peptide or a larger precursor encoding the 9-mer immunogenic peptide identified by any of the methods described herein. In some embodiments, the immunogenic composition contains two or more immunogenic tumor-specific mutant peptides. In some embodiments, the immunogenic composition contains two or more immunogenic cancer-specific mutant peptides.

The present application also provides an immunogenic composition comprising at least one nucleic acid encoding tumor or cancer specific immunogenic peptide, or one nucleic acid encoding a larger precursor containing the 9-mer mutant immunogenic peptide identified by any of the methods described herein. In some embodiments, the immunogenic composition comprising a nucleic acid encoding two or more (up to about 20) tumor-specific mutant immunogenic peptides. In some embodiments, the immunogenic composition comprising a nucleic acid encoding two or more (up to about 20) cancer-specific mutant immunogenic peptides. In other embodiments, the immunogenic composition can be composed of a mixture of immunogenic peptides, or a DNA encoding one or more immunogenic peptides, or a RNA encoding one or more immunogenic peptides.

### BRIEF DESCRIPTION OF THE FIGURES

FIG. 1. Steps to identity immunogenic peptides from cancer tissues.

FIG. 2. Steps for the creation of classification models for predicting TCR-binding peptides derived from normal and cancer tissues.

FIG. 3a-b. (a) Binding affinity distribution of immunogenic and non-immunogenic peptides, (b) Distribution of peptide with >=500 nM and <500 nM.

FIG. 4. A schematic of the steps used for creating the classification models to separate TCR-binding peptides (immunogenic) from those that did not bind TCR (non-immunogenic).

FIG. 5a-b. (a) Sensitivity and specificity of the 500 training/test instances using J4.8 classification approach, (b) ROC curve from the ensemble classifier.

FIG. 6a-b. (a) Sensitivity and specificity of the 433 classifier instances using J4.8 classification approach, (b) The ROC curve for the 433 classifiers (colored in RED), 45 classifiers (colored in Blue).

FIG. 7a-c. Features to identify selected peptides. (a) Number of features that define occupancy of amino acids at each position of the 9-mer peptide. (b) Number of features that define hydrophobicity and helix/turn properties of amino acids. (c) Enrichment of amino acids with helix-turn and hydrophobicity properties at each position of the 9-mer peptides.

FIG. 8. Shows a schematic representation of the assay.

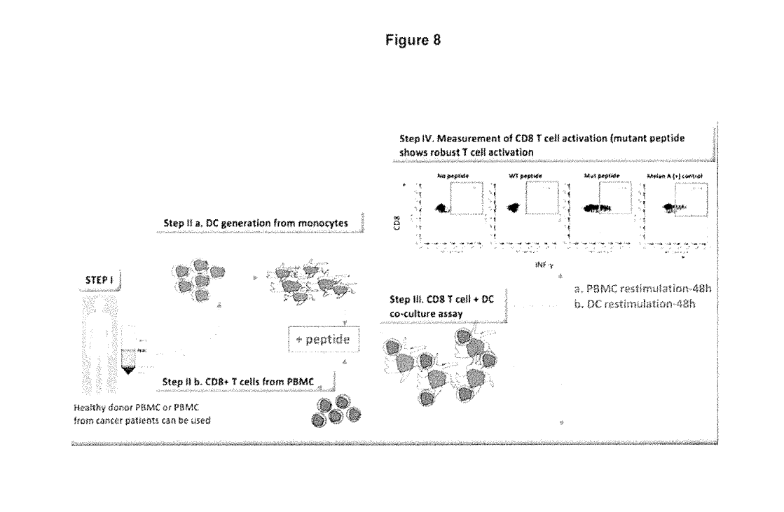

FIG. 9. The data presented here shows a validated neoantigen restricted to HLA-A*02.01 as evidenced by elevated levels of CD8 T cell activation markers, INF-γ and CD69 in flow cytometric based assays. Naïve human CD8 T cells specific for the HLA-A*02.01-restricted epitopes showed a positive response to a colorectal cancer derived mutant peptide over a wild-type (control) peptide when stimulated with peptide-pulsed allogeneic DCs. Melan-A (26-35L, positive control) is used as a positive control.

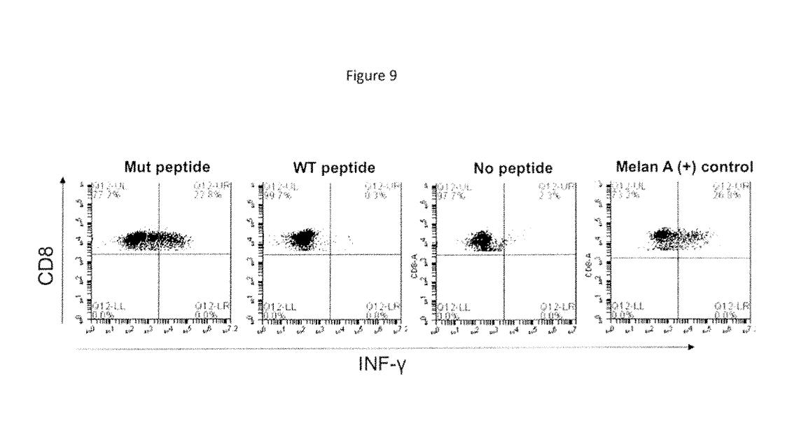

### DETAILED DESCRIPTION OF THE INVENTION

Unless defined otherwise, all technical and scientific terms used herein have the same meaning as is commonly understood by one of ordinary skill in the art to which this invention belongs. All patents, applications, published applications and other publications referred to herein are incorporated by reference in their entirety.

As used in the description of the invention and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

As used herein, “at least one” is intended to mean “one or more” of the listed elements.

Except where noted otherwise, capitalized and non-capitalized forms of all terms fall within each meaning.

Unless otherwise indicated, it is to be understood that all numbers expressing quantities, ratios, and numerical properties of ingredients, reaction conditions, and so forth used in the specification and claims are contemplated to be able to be modified in all instances by the term “about.” As used herein, the term “about” when used before a numerical designation, e.g., temperature, time, amount, concentration, and such other, including a range, indicates approximations which may vary by (+) or (−) 10%, 5% or 1%.

As used herein, the term “substantially free”includes being free of a given substance or cell type or nearly free of that substance or cell type, e.g. having less than about 1% of the given substance or cell type.

As used in this application, “cancer-specific mutant peptide” refers to a peptide that comprises at least one mutated amino acid present in the cancer tissue and absent in the normal tissue. The “cancer immunogenic peptide or tumor immunogenic peptide” refers to a peptide that comprises at least one mutated amino acid that is present in the cancer tissue and absent in the normal tissue and is capable of binding TCR and evoking a T cell response in the individual. The immunogenic peptides of the invention which are selected by the methods of the invention may be synthesized or expressed to be part of a larger polypeptide tumor vaccine. Alternatively, the nucleic acid encoding the immunogenic peptide of the invention may be used as part of a larger tumor vaccine. Cancer-tumor immunogenic peptides can arise from i) proteins altered in amino acid sequence in which one or more amino acids are altered, which may be arranged in a sequence or distributed randomly across the length of the protein; ii) proteins translated from fusion genes; iii) proteins produced from splice variants or from mutations in splicing sites, which results in the introduction of intronic region or part of an intronic region in frame with the protein coding sequence or exclusion of part or whole exon(s) resulting in an altered protein with new sequence at the site of the lost exonic region; iv) Proteins produced from insertions and/or deletions of nucleotides that cause frameshift in the protein coding sequence resulting in the introduction of one or more amino acids absent in the normal protein [22]; or vi) protein arising from loss of stop codons (stop loss) that adds additional amino acids at the end of the protein [23].

An “immunogenic peptide” in this application refers to a mutant peptide capable of transducing a signal CD4⁺and CD8⁺T cells. An “immunogenic peptide used as a vaccine” in this application refers to a longer peptide of length ranging from about >11-mer up to about 50-mer containing within the longer peptide the minimal sequence of the immunogenic peptide.

A “variant coding sequence” in this application refers to a nucleic acid sequence (DNA or RNA) from a cancer sample containing one or more variant nucleotides compared to the sequence in the reference normal sample. The sequence variation results in a change in the amino acid sequence of the protein encoded by the nucleic acid sequence.

The “expressed variant coding sequence” in this application refers to a nucleic acid sequence derived from RNA expressed in the tumor or cancer tissue of the individual.

A nucleic acid sequence “encoding” a peptide refers to a sequence of DNA or RNA containing the coding sequence of the peptide.

The “conceptual translation or in silico translation of the coding sequences” refers to translation of the coding sequence of a nucleic acid to amino acid sequence based on a codon table specifying amino acids, so as to obtain peptide or protein with a defined amino acid sequence. A computer and software may be used to perform the “conceptual translation or in silico translation of the coding sequences.”

The “genetically altered protein(s) expressed by the mammalian tumor cell or the mammalian tumor tissue” refers to altered or mutated protein(s) reflective of changes in the genetic material present in the mammalian tumor cell or tissue.

The “class I HLA or equivalent” is class I MHC molecules of human or any other mammalian species.

The “HLA-binding neoepitope” in the context of class I HLA molecules refers to a peptide sequence of 8-11 amino acids in length in which one or more amino acids are mutated, which can bind or is predicted to bind to specific class I HLA molecules. The “HLA-binding epitope” in the context of class I HLA molecules refers to peptides containing mutated or non-mutated amino acids. For example, the HLA may be a class I HLA molecules.

The “MHC-binding neo-epitope” in the context of class I MHC molecules refers to a peptide sequence of 8-11 amino acids in length in which one or more amino acids are mutated, which can bind or is predicted to bind to specific class I MHC molecules. The “MHC-binding epitope” in the contest of class I MHC molecules refers to peptides containing mutated or non-mutated amino acids.

The “HLA-binding neo-epitope” in the context of class II HLA molecules refers to a peptide sequence of 13-21 amino acids in length in which one or more amino acids are mutated, which can bind or is predicted to bind to specific class II HLA molecules. The “HLA-binding epitope” in the contest of class II HLA molecules refers to peptides containing mutated or non-mutated amino acids.

The “MHC-binding neo-epitope” in the context of class II MHC molecules refers to a peptide sequence of 13-21 amino acids in length in which one or more amino acids are mutated, which can bind or is predicted to bind to specific class II MHC molecules. The “MHC-binding epitope” in the contest of class II MHC molecules refers to peptides containing mutated or non-mutated amino acids.

“T-cell neo-epitopes” refers to a peptide in which one or more amino acids are mutated, which can bind or is predicted to bind to T-cell receptor of CD8+T-cell or CD4+T-cell.

An “immunogenic peptide” is by definition a “HLA-binding neoepitope” or “HLA-binding epitope”. However, all HLA-binding neoepitopes or HLA-binding epitopes may not be “immunogenic peptides”.

The “peptide precursor” is a protein present in the cancer tissue that contains the peptide of interest. Multiple “peptide precursors” can contain the peptide of interest.

A “disease tissue” in this application refers to tumor or cancer tissue from human or mice.

A “tumor” or “neoplasm” is an abnormal growth of tissue whether benign or malignant.

A “cancer” may be a malignant tumor or malignant neoplasm. Cancer refers to any one of cancer, tumor growth, cancer of the colon, breast, bone, brain and others (e.g., osteosarcoma, neuroblastoma, colon adenocarcinoma), chronic myelogenous leukemia (CML), acute myeloid leukemia (AML), acute promyelocytic leukemia (APL), cardiac cancer (e.g., sarcoma, myxoma, rhabdomyoma, fibroma, lipoma and teratoma); lung cancer (e.g., bronchogenic carcinoma, alveolar carcinoma, bronchial adenoma, sarcoma, lymphoma, chondromatous hamartoma, mesothelioma); various gastrointestinal cancers (e.g., cancers of esophagus, stomach, pancreas, small bowel, and large bowel); genitourinary tract cancer (e.g., kidney, bladder and urethra, prostate, testis; liver cancer (e.g., hepatoma, cholangiocarcinoma, hepatoblastoma, angiosarcoma, hepatocellular adenoma, hemangioma); bone cancer (e.g., osteogenic sarcoma, fibrosarcoma, malignant fibrous histiocytoma, chondrosarcoma, Ewing's sarcoma, malignant lymphoma, multiple myeloma, malignant giant cell tumor chordoma, osteochronfroma, benign chondroma, chondroblastoma, chondromyxofibroma, osteoid osteoma and giant cell tumors); cancers of the nervous system (e.g., of the skull, meninges, brain, and spinal cord); gynecological cancers (e.g., uterus, cervix, ovaries, vulva, vagina); hematologic cancer (e.g., cancers relating to blood, Hodgkin's disease, non-Hodgkin's lymphoma); skin cancer (e.g., malignant melanoma, basal cell carcinoma, squamous cell carcinoma, Karposi's sarcoma, moles dysplastic nevi, lipoma, angioma, dermatofibroma, keloids, psoriasis); and cancers of the adrenal glands (e.g., neuroblastoma).

Examples of tumors include colorectal cancer, osteosarcoma, non-small cell lung cancer, breast cancer, ovarian cancer, glial cancer, solid tumors, metastatic tumor, acute lymphoblastic leukemia, acute myelogenous leukemia, adrenocortical carcinoma, Kaposi sarcoma, lymphoma, anal cancer, astrocytomas, basal cell carcinoma, bile duct cancer, bladder cancer, bone cancer, brain tumor, breast cancer, bronchial tumor, cervical cancer, chronic lymphocytic leukemia, chronic myelogenous leukemia, chronic myeloproliferative disorders, colon cancer, colorectal cancers, ductal carcinoma in situ, endometrial cancer, esophageal cancer, eye cancer, intraocular, retinoblastoma, metastatic melanoma, gallbladder cancer, gastric cancer, gastrointestinal carcinoid tumor, gastrointestinal stromal tumors, glioblastoma, glioma, hairy cell leukemia, head and neck cancer, hepatocellular carcinoma, hepatoma, Hodgkin lymphoma, hypopharyngeal cancer, Langerhans cell histiocytosis, laryngeal cancer, lip and oral cavity cancer, liver cancer, lobular carcinoma in situ, lung cancer, non-small cell lung cancer, small cell lung cancer, lymphoma, AIDS-related lymphoma, Burkitt lymphoma, non-Hodgkin lymphoma, cutaneous T-cell lymphoma, melanoma, squamous neck cancer, mouth cancer, multiple myeloma, myelodysplastic syndromes, myelodysplastic/myeloproliferative neoplasms, nasal cavity and paranasal sinus cancer, nasopharyngeal cancer, neuroblastoma, oral cavity cancer, oropharyngeal cancer, osteosarcoma, ovarian cancer, pancreatic carcinoma, papillary carcinomas, parathyroid cancer, pharyngeal cancer, pheochromocytoma, pineal parenchymal tumors, pineoblastoma, pituitary tumor, pleuropulmonary blastoma, primary central nervous system lymphoma, prostate cancer, rectal cancer, renal cell cancer, salivary gland cancer, sarcoma, Ewing sarcoma, soft tissue sarcoma, squamous cell carcinoma, Sezary syndrome, skin cancer, Merkel cell carcinoma, testicular cancer, throat cancer, thymoma, thymic carcinoma, thyroid cancer, urethral cancer, endometrial cancer, uterine cancer, uterine sarcoma, vaginal cancer, vulvar cancer, Waldenstrom macroglobulinemia, and Wilms tumor. In one embodiment, the tumor is a glioma. In one embodiment, the tumor is a tumor other than a glioma.

For example, an inhibition of growth of a cancer cell means that the rate of growth of a cancer cell that has been treated with a peptide of the invention is 5-fold, 10-fold, 20-fold, 30-fold, 40-fold, 50-fold, 100-fold, or more, less than that of a cancer cell that has not been treated with a peptide of the invention. As used herein, “inhibition” as it refers to the rate of growth of a cancer cell that has been treated with a peptide of the invention also means that the rate is 90%, 80%, 70%, 60%, 50%, 40%, 30%, 25%, 20%, 15%, 10%, 5% or less, lower than the rate of growth of a cancer cell that has not been treated with a peptide of the invention.

An inhibition of growth of a cancer cell also means that the number or growth of cancer cells that have been treated with a peptide of the invention is 5-fold, 10-fold, 20-fold, 30-fold, 40-fold, 50-fold, 100-fold, or more, less than the number or growth of cancer cells that have not been treated with a peptide of the invention. As used herein, “inhibition” as it refers to the rate of growth of a cancer cell also means that the number or growth of cancer cells that have been treated with a peptide of the invention is 90%, 80%, 70%, 60%, 50%, 40%, 30%, 25%, 20%, 15%, 10%, 5% or less, lower than the growth or number of cancer cells that have not been treated with a peptide of the invention.

As used herein, “cancer” may be used interchangeably with “tumor,” and vice versa, except when expressly or inherently prohibited. Similarly, “MHC” may be used interchangeably with “HLA,” and vice versa, except when expressly or inherently prohibited.

The term “unmutated or wild-type peptide” refers to a peptide derived from normal or healthy tissue cells or tissue. Normal or healthy cells or tissue are free of disease, and in the context of the invention, free of tumor/cancer tissue or cells. Unlike cancer-specific mutant peptide, tumor peptide variant(s) or cancer peptide variant(s), which are mutant or altered peptide specific to cancer or tumor cells or tissues and not present in non-tumor/cancer cells or tissue, the “unmutated or wild-type peptide” may be present in cancer or tumor cells or tissue.

As used herein, the terms “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the present disclosure. “Consisting of” shall mean excluding more than trace elements of other ingredients and substantial method steps. Embodiments defined by each of these transition terms are within the scope of the present disclosure.

### Methods of the Invention

The invention describes a method for identifying immunogenic peptides from all genetically altered proteins derived from mammalian cancer samples using a high throughput approach. An accurate high throughput platform for the detection of immunogenic epitopes is critical for clinical translation. The immunogenic peptides can be administered as personal cancer vaccines to individuals affected by the disease in the form of peptides, or as nucleotide-based precursors (e.g., DNA or RNA). The immunogenic peptides can have other applications in identifying specific TCR sequences that engage with the peptide, leading to the development of engineered T cells or CAR-T cells. Additionally, the immunogenic peptides can be used for developing TCR-mimetic reagents to target tumor cells. The methods described herein are useful in personalized cancer immunotherapy space for the treatment of individual cancer patients.

Thus, the present invention in one aspect provides a method of identifying cancer-specific mutant immunogenic peptide from the disease tissue of the individual by combining sequence-specific variant detection method with methods to determine immunogenicity of the peptides.

In another aspect, the present invention provides a method of identifying cancer-specific immunogenic peptides that bind T-cell receptor (TCR).

Also provided are enablement steps useful to practice the invention. Further included are a list of immunogenic peptides from cancer mutations detected by next generation sequencing, cancers presenting such peptides and nucleic acids encoding such peptides identified.

The invention provides methods of selecting cross species cancer vaccines from genetically altered proteins expressed by mouse and human cancer cells and/or tissues. In one embodiment, the method comprises (a) calculating the probability of HLA binding with optimal processing sites from a library of mutant cancer peptides; (b) calculating the probability of TCR binding to generate a T-cell response; and selecting the mutant cancer peptides having the highest probability so calculated from step (a) that can modulate the immune response of a mouse and a human, when challenged with the mutant cancer peptide thereby selecting cross species cancer vaccines; wherein the mouse and human subjects carry the same mutation and express the same HLA molecule that binds the mutant cancer peptide.

In accordance with the practice of the invention the tumor may be derived from any cancer. Examples of cancer cells or tissues include, but are not limited to, cancers of the Breast, Lung, Head & Neck, Skin, Ovary, Pancreatic, Liver, Brain, Prostate, Cervical Thyroid, Bone and Stomach.

The invention further provides methods of selecting mammalian tumor vaccine(s) from genetically altered protein(s) expressed by a mammalian tumor cell or a mammalian tumor tissue from a subject. In one embodiment of the invention, the method comprises the step of obtaining a sample from the subject. The sample may be directly processed as soon as it is obtained or the sample may be stored for a period of time before it is processed in accordance with the invention. The sample obtained from the subject may be cultured in vitro or used to produce cell line before processing in accordance with the invention. The method further comprises the step of identifying the genetically altered protein(s) expressed by the mammalian tumor cell or the mammalian tumor tissue in the sample through nucleic acid sequence(s) encoding the altered protein(s). Additionally, the method includes the step of producing peptide fragment(s) comprising at least one amino acid mutation from the genetically altered protein(s) so identified, so as to obtain peptide variant(s) associated with the mammalian tumor cell or the mammalian tumor tissue. In one embodiment, the peptide fragments are produced in silico using a sliding window method for a fixed or defined peptide length with one amino acid step producing a series of overlapping peptides of a pre-defined length with any mutant amino acid occupying different amino acid position in the series of peptides produced by the sliding window method.

Further, the method additionally comprises the step of selecting the peptide variant(s) which binds T-cell receptor (TCR). In one embodiment, this step comprises i) selecting the peptide variant(s) with a pre-defined length; ii) characterizing the peptide variant(s) in silico by selecting and matching features associated with an amino acid at each position of the peptide with selected pre-defined features for each position of peptides recognized by TCR associated with either CD8+ T-cell or CD4+ T-cell, so as to obtain predictive ability of the peptide variant(s) to interact with the TCR; iii) selecting the peptide variant(s) in step (ii) based on predicted ability of the peptide variant(s) to interact with the TCR, so as to be an immunogenic peptide that may or can serve as a mammalian tumor vaccine(s). Basis for mammalian tumor vaccine(s) using peptide variant(s) identified and selected by the methods of the invention require lengthening the selected peptide variant(s) such that following vaccination the lengthened selected peptide variant(s) is taken up by antigen-presenting cells, processed to the size of the selected peptide variant(s) (before lengthening) and displayed by antigen-presenting cells. In one embodiment, the antigen-presenting cells are professional antigen-presenting cells. In an embodiment, the professional antigen-presenting cells are dendritic cells, macrophages and B lymphocytes. Merely as examples, the peptide variant(s) so selected with a pre-defined length may be a peptide fragment of 8, 9, 10, or 11 amino acids in length. Such a peptide with 8 to 11 amino acids is bound and displayed by class I MHC molecules or class I HLA molecules for TCR binding or interaction. In a preferred embodiment, the peptide variant(s) may be a peptide fragment of 9, 10 or 11 amino acids in length. For example, in a more preferred embodiment, the peptide variant(s) may be a peptide fragment of 9 amino acids in length. In another embodiment, the peptide variant(s) may be a peptide fragment of 13, 14, 15, 16, 17, 18, 19, 20 or 21 amino acids in length. Such a peptide with 13 to 21 amino acids is bound and displayed by class II MHC molecules or class II HLA molecules for TCR binding or interaction. In a preferred embodiment, the peptide variant(s) may be a peptide fragment of 14, 15, 16 or 17 amino acids in length. For example, in a more preferred embodiment, the peptide variant(s) may be a peptide fragment of 16 or 17 amino acids in length. In an embodiment of the invention, the pre-defined length of the peptide variant(s) may vary with the proviso that the size of the peptide variant(s) permits interaction with MHC class I protein(s). In one embodiment, the interaction with MHC class I proteins is a binding reaction that permits display of the peptide variant by MHC class I protein(s). Alternatively, in another embodiment, the pre-defined length of the peptide variant(s) may vary with the proviso that the size of the peptide variant(s) permits interaction with MHC class II protein(s). In one embodiment, the interaction with MHC class II proteins is a binding reaction that permits display of the peptide variant by MHC class II protein(s).

In one embodiment, the immunogenic peptide may be selected further by its ability to bind MHC class-I or class-II protein(s) comprising: a) calculating the binding affinity of the immunogenic peptide to MHC class-I or class-II protein(s); and b) further selecting a set of peptide variant(s) from the previous step where the binding affinity of the unmutated or wild-type peptide is weaker than the variant or the mutated peptide for MHC class-I or class-II protein(s).

In another embodiment, the step of selecting mammalian tumor vaccine(s) includes selecting immunogenic peptide variant(s) for vaccination.

In accordance with the practice of the invention, the mammalian tumor cell or the mammalian tumor tissue may be derived from a mammal, wherein the mammal is selected from the group consisting of human, mouse, rat, cat, dog, bovine, pig, sheep, goat, cow, horse, hamster, guinea pig, rabbit, mink, monkey, chimpanzee, and ape. In one embodiment, the mammalian tumor cell or the mammalian tumor tissue is derived from a mammal, wherein the mammal is a mouse. In one embodiment, the mammalian tumor cell or the mammalian tumor tissue is derived from a mammal, wherein the mammal is a rat. In another embodiment, the mammalian tumor cell or the mammalian tumor tissue is derived from a mammal, wherein the mammal is a human.

In yet another embodiment of the invention, identifying the genetically altered protein(s) expressed by the mammalian tumor cell or the mammalian tumor tissue through nucleic acid sequence(s) encoding the altered protein(s) may comprise (a) the identifying tumor variants from transcriptome analysis of the mammalian tumor cell or mammalian tumor tissue corresponding to protein coding and protein non-coding sequences; and (b) performing conceptual translation or in silico translation of the coding sequences in step (a) so as to identify the genetically altered protein(s) expressed by the mammalian tumor cell or the mammalian tumor tissue.

For example, identifying tumor variants from transcriptome analysis of the mammalian tumor cell or mammalian tumor tissue may comprise the steps of a) determining nucleotide sequence of transcripts produced by the mammalian tumor cell or mammalian tumor tissue; and b) comparing the determined nucleotide sequence of transcripts in (a) with a reference nucleotide sequence of transcripts produced by mammalian non-tumor cell or mammalian non-tumor tissue, so as to identify nucleotide sequence changes in the protein coding and protein non-coding sequences.

In one embodiment, the reference nucleotide sequence of transcripts produced by mammalian non-tumor cell or mammalian non-tumor tissue may be obtained from a publically available database. Alternatively, the reference nucleotide sequence of transcripts produced by mammalian non-tumor cell or mammalian non-tumor tissue may be obtained from a clonal population of a normal culture cell or a collection of clonal population of normal cultured cells, a normal tissue or a collection of normal tissues, a collection of normal tissues from different organ systems, an individual or a collection of individuals, a collection of individuals with similar genetic background, an individual of the same sex or a collection of individuals of the same sex, an individual of a different sex or a collection of individuals of a different sex, an individual of a particular age group or a collection of individuals of a particular age group, a collection of individuals from different stages of development, an individual or group of individuals of a species or sub-species or a combination thereof, wherein normal refers to absence of tumor or tumor material in specimen used to determine the reference nucleotide sequence of transcripts. In one embodiment, the different stages of development may be selected from the group consisting of embryo, fetus, neonate, infant, toddler, early childhood, child, preadolescence, adolescence, adult, middle age and old age and equivalent stages thereof.

For example, the collection of individuals with similar genetic background may be selected from the group consisting of a group of inbred animals or individuals, a collection of family members, a collection of individuals within a family tree, a collection of individuals breeding within a geographic restricted region, a collection of individuals of the same ethnicity and a collection of individuals of the same race.

For example, the species or sub-species may belong to the genus selected from any of Homo, Mus and Rattus. In one embodiment, the species is Homo sapiens such as the sub-species is Homo sapiens. In another embodiment, the species is any of Mus musculus, Mus booduga, Mus caroli, Mus cervicolor, Mus cookie, Mus cypriacus, Mus famulus, Mus fragilicauda, Mus macedonicus, Mus nitidulus, Mus spicilegus, Mus spretus, Mus terricolor, Mus crociduroides, Mus mayori, Mus pahari, Mus vulcani, Mus baoulei, Mus bufo, Mus callewaerti, Mus goundae, Mus haussa, Mus indutus, Mus mahomet, Mus mattheyi, Mus minutoides, Mus musculoides, Mus neavi, Mus orangiae, Mus oubanguii, Mus setulosus, Mus setzeri, Mus sorella, Mus tenellus, Mus triton, Mus fernandoni, Mus phillipsi, Mus platyhrix, Mus saxicola, Mus shortridgei or Mus lepidoides. In this case, the sub-species may be any of Mus musculus, Mus musculus molossinus, Mus musculus castaneus or Mus musculus domesticus.

In yet a further example, the species may be any of Rattus norvegicus, Rattus, Rattus annandalei, Rattus enganus, Rattus everetti, Rattus exulans, Rattus hainaldi, Rattus hoogerwerfi, Rattus korinchi, Rattus macleari, Rattus montanus, Rattus morotaiensis, Rattus nativiatis, Rattus ranjiniae, Rattus sanila, Rattus stoicus, Rattus timorensis, Rattus nitidus, Rattus pyctoris, Rattus turkestanicus, Rattus adustus, Rattus andamanesis, Rattus argentiventer, Rattus baluensis, Rattus blangorum, Rattus burrus, Rattus hoffmanni, Rattus koopmani, Rattus losea, Rattus lugens, Rattus mindorensis, Rattus mollicomulus, Rattus osgoodi, Rattus palmarum, Rattus satarae, Rattus simalurensis, Rattus tanezumi, Rattus tawitawiensis, Rattus tiomanicus, Rattus bontanus, Rattus foramineus, Rattus marmosurus, Rattus pelurus, Rattus salocco, Rattus xanthurus, Rattus arfakiensis, Rattus arrogans, Rattus elaphinus, Rattus feliceus, Rattus giluwensis, Rattus jobiensis, Rattus leucopus, Rattus mordax, Rattus niobe, Rattus novaeguineae, Rattus omichlodes, Rattus pococki, Rattus praetor, Rattus richardsoni, Rattus steini, Rattus vandeuseni, Rattus verecundus, Rattus colletti, Rattus fuscipes, Rattus lutreolus, Rattus sordidus, Rattus tunneyi or Rattus villosissimus.

In yet another embodiment, the reference nucleotide sequence of transcripts produced by mammalian non-tumor cell or mammalian non-tumor tissue may be a composite of nucleotide sequence of transcripts from multiple normal specimen or sources, wherein normal refers to absence of tumor or tumor material in specimen or sources.

In a further embodiment of the invention, the step of identifying the genetically altered protein(s), may further comprise performing genomic analysis for tumor variants in the sequence of the genome present in the mammalian tumor cell or the mammalian tumor tissue but absent or deficient in the mammalian non-tumor cell or the mammalian non-tumor tissue. Merely by way of example, the genomic analysis for tumor variants may include determining nucleotide sequence of the genome or exome.

In another embodiment of the invention, the genetically altered protein(s) expressed by the mammalian tumor cell or the mammalian tumor tissue may be absent or deficient in the mammalian non-tumor cell or the mammalian non-tumor tissue.

In a further embodiment of the invention, the step of producing peptide fragment(s) may comprise at least one amino acid mutation from each genetically altered protein, so as to obtain peptide variant(s) associated with the mammalian tumor cell or the mammalian tumor tissue, the step comprises: defining length of the peptide fragment(s) to be produced from the genetically altered protein; and producing in silico peptide fragment(s) of the pre-defined length at a site of alteration in the protein comprising at least one mutated amino acid of the genetically altered protein.

In another embodiment of the invention, the method comprises identifying a set of tumor variant(s) from a sample comprising mammalian tumor cell or the mammalian tumor tissue from a subject. In accordance with the practice of the invention, in one embodiment, each variant in the genomic sequence corresponds to protein coding or protein non-coding sequence comprising the steps of determining nucleic acid sequence of tumor genetic material and comparing to non-tumor reference sequence to identify tumor variant(s). In an embodiment, the method further comprises the step of detecting the tumor variant(s) expressed by the mammalian tumor cell or the mammalian tumor tissue resulting in an alteration in mRNA sequence and sequence of protein translated from the mRNA. Additionally, the method comprises the step of translating in silico the mRNA so identified in step (b) to obtain genetically altered protein(s) produced or expected to be produced by the mammalian tumor cell or the mammalian tumor tissue. Further, the method comprises generating peptide fragment(s) of a pre-defined length in silico from the altered protein(s), after which, the method further provides the steps of identifying peptide variant(s) of the mammalian tumor cell or the mammalian tumor tissue which is not associated with mammalian non-tumor cell or tissue; predicting immunogenicity of the peptide variant(s) comprising a step of in silico assessment of peptide ability to interact with T-cell receptor; and selecting immunogenic peptide variant(s) based on the predicted ability of the peptide variant(s) to interact with the TCR, which may be used as a basis for mammalian tumor vaccine(s). Basis for mammalian tumor vaccine(s) using peptide variant(s) identified and selected by the methods of the invention requires lengthening the selected peptide variant(s) such that following vaccination, the lengthened selected peptide variant(s) is taken up by antigen-presenting cells, processed to the size of the selected peptide variant(s) (before lengthening) and displayed by antigen-presenting cells. In one embodiment, the antigen-presenting cells are professional antigen-presenting cells. In an embodiment, the professional antigen-presenting cells are dendritic cells, macrophages and B lymphocytes.

In another embodiment of the invention, the immunogenic peptide may be further selected by its potential or ability to be produced inside the cell by processes comprising the steps of determining the action of proteases which are part of the proteasomal or immunoproteasomal complexes, based on the probability that the processing event of the altered protein(s) will produce the immunogenic peptide so selected; and determining the entry of the immunogenic peptide into the endoplasmic reticulum compartment by binding to peptide transporters expressed on the surface of the compartment. For example, the peptide transporter may be a transporter associated with antigen processing (TAP) comprising TAP1 and TAP2.

In accordance with the practice of the invention, the methods of the invention may further comprise predicting immunogenicity of peptide variant(s) derived from the mammalian tumor cell or the mammalian tumor tissue, and optionally, immunogenicity of corresponding non-variant peptide from mammalian non-tumor cell or the mammalian non-tumor tissue.

In another embodiment of the invention, the immunogenic peptide may be further selected by its potential or ability to be produced inside the cell by processes comprising: a) determining action of proteases, which are part of the lysosome and/or endosomal compartments, based on the probability that the processing event of the altered protein(s) will produce the immunogenic peptide so selected; and b) determining the fusion of the endosomal and/or lysosomal vesicles with Golgi-derived vesicles to permit loading of the immunogenic peptide onto MHC class II proteins.

In one embodiment of the invention, the length of the peptide fragment(s) to be produced from the genetically altered protein or the peptide fragment(s) of the pre-defined length is 8 amino acids or more. In another embodiment, the length of the peptide fragment(s) to be produced from the genetically altered protein or peptide fragment(s) of the pre-defined length is less than 18 amino acids.

In yet a further embodiment, the length of the peptide fragment(s) to be produced from the genetically altered protein or the peptide fragment(s) of the pre-defined length may be a length that permits binding by MHC class I protein. For example, the length that permits binding by MHC class I protein may be selected to be 8, 9, 10, or 11 amino acids long. In another example, the length that permits binding by MHC class II protein is selected to be 13, 14, 15, 16, 17, 18, 19, 20 or 21 amino acids long.

In another embodiment, the length of the peptide fragment(s) to be produced from the genetically altered protein or the peptide fragment(s) of the pre-defined length is about 9, 10 or 11 amino acids long. In a specific example, the length of the peptide fragment(s) to be produced from the genetically altered protein or the peptide fragment(s) of the pre-defined length is 9 amino acids long.

In yet another embodiment, the length of the peptide fragment(s) further supports interaction with the TCR of CD8+ T-cell or CD4+ T-cell.

In still another embodiment, the interaction with the TCR of CD8+ T-cell or CD4+ T-cell results in a complex comprising the peptide, MHC class I protein and TCR of CD8+ T-cell, or alternatively, the peptide, MHC class II protein and TCR of CD4+ T-cell.

In an additional embodiment, interaction with the TCR of CD8+ T-cell or CD4+ results in a complex comprising the peptide, MHC class I protein and TCR of CD8+ T-cell, or alternatively, the peptide, MHC class II protein and TCR of CD4+ T-cell.

Also, in another embodiment, the mammalian tumor cell is a cell of a mammalian cell line derived from the tumor of a mammal. Merely by way of example, the mammal is selected from the group of human, mouse, rat, cat, dog, bovine, pig, sheep, goat, cow, horse, hamster, guinea pig, rabbit, mink, monkey, chimpanzee, and ape. In one embodiment, the mammal is a mouse or a human. In another embodiment, the tumor is a cancer. In yet a further embodiment, the mammalian tumor cell is a cell of a mouse cancer cell line. In a further still embodiment, the mammalian tumor cell is a cell of a human cancer cell line. Further, the mammalian tumor cell or mammalian tumor tissue may be present in or derived from a mouse or human subject.

Additionally, in accordance with the practice of the invention, the features associated with an amino acid at each position of the peptide may be physicochemical and/or biological properties of the amino acid. For example, each physicochemical and/or biological property of an amino acid may be assigned a numerical value within the context of other numerical values assigned to other amino acids.

Suitable examples of pre-defined features in accordance with the invention, include, but are not limited to, one of more of alpha-CH chemical shifts, hydrophobicity index (1), signal sequence helical potential, membrane-buried preference parameters, conformational parameter of inner helix, conformational parameter of beta-structure, conformational parameter of beta-turn, average flexibility indices, residue volume, information value for accessibility—average fraction 35%, information value for accessibility—average fraction 23%, retention coefficient in TFA, retention coefficient in HFBA, transfer free energy to surface, apparent partial specific volume, alpha-NH chemical shifts, alpha-CH chemical shifts, spin-spin coupling constants 3JHalpha-NH, normalized frequency of alpha-helix, normalized frequency of extended structure, steric parameter, polarizability parameter, free energy of solution in water—kcal/mole, Chou-Fasman parameter of the coil conformation, a parameter defined from the residuals obtained from the best correlation of the Chou-Fasman parameter of beta-sheet, number of atoms in the side chain labelled 1+1, number of atoms in the side chain labelled 2+1, number of atoms in the side chain labelled 3+1, number of bonds in the longest chain, a parameter of charge transfer capability, a parameter of charge transfer donor capability, average volume of buried residue, residue accessible surface area in tripeptide, residue accessible surface area in folded protein, proportion of residues 95% buried, proportion of residues 100% buried, normalized frequency of beta-turn—1, normalized frequency of alpha-helix, normalized frequency of beta-sheet, normalized frequency of beta-turn—2, normalized frequency N-terminal helix, normalized frequency of C-terminal helix, normalized frequency of N-terminal non helical region, normalized frequency of C-terminal non helical region, normalized frequency of N-terminal beta-sheet, normalized frequency of C-terminal beta-sheet, normalized frequency of N-terminal non beta region, normalized frequency of C-terminal non beta region, frequency of the 1st residue in turn, frequency of the 2nd residue in turn, frequency of the 3rd residue in turn, frequency of the 4th residue in turn, normalized frequency of the 2nd and 3rd residues in turn, normalized hydrophobicity scales for alpha-proteins, normalized hydrophobicity scales for beta-proteins, normalized hydrophobicity scales for alpha+beta-proteins, normalized hydrophobicity scales for alpha/beta-proteins, normalized average hydrophobicity scales, partial specific volume, normalized frequency of middle helix, normalized frequency of beta-sheet, normalized frequency of turn, size, amino acid composition, relative mutability, membrane preference for cytochrome b: MPH89, average membrane preference: AMP07, consensus normalized hydrophobicity scale, solvation free energy, atom-based hydrophobic moment, direction of hydrophobic moment, molecular weight, melting point, optical rotation, pK-N, pK-C, hydrophobic parameter pi, graph shape index, smoothed upsilon steric parameter, normalized van der Waals volume, STERIMOL length of the side chain, STERIMOL minimum width of the side chain, STERIMOL maximum width of the side chain, N.M.R. chemical shift of alpha-carbon, localized electrical effect, number of hydrogen bond donors, number of full nonbonding orbitals, positive charge, negative charge, pK-a(RCOOH), helix-coil equilibrium constant, helix initiation parameter at position i−1, helix initiation parameter (at position i, i+1, and i+2), helix termination parameter (at position j−2, j−1, and j), helix termination parameter at position j+1, partition coefficient, alpha-helix indices, alpha-helix indices for alpha-proteins, alpha-helix indices for beta-proteins, alpha-helix indices for alpha/beta-proteins, beta-strand indices, beta-strand indices for beta-proteins, beta-strand indices for alpha/beta-proteins, aperiodic indices, aperiodic indices for alpha-proteins, aperiodic indices for beta-proteins, aperiodic indices for alpha/beta-proteins, hydrophobicity factor, residue volume, composition, polarity, volume, partition energy, hydration number, hydrophilicity value, heat capacity, absolute entropy, entropy of formation, normalized relative frequency of alpha-helix, normalized relative frequency of extended structure, normalized relative frequency of bend, normalized relative frequency of bend R, normalized relative frequency of bend S, normalized relative frequency of helix end, normalized relative frequency of double bend, normalized relative frequency of coil, average accessible surface area, percentage of buried residues, percentage of exposed residues, ratio of buried and accessible molar fractions, transfer free energy, hydrophobicity (1), pK (—COOH), relative frequency of occurrence, relative mutability, amino acid distribution, sequence frequency, average relative probability of helix, average relative probability of beta-sheet, average relative probability of inner helix, average relative probability of inner beta-sheet, flexibility parameter for no rigid neighbors, flexibility parameter for one rigid neighbor, flexibility parameter for two rigid neighbors, Kerr-constant increments, net charge, side chain interaction parameter (1), side chain interaction parameter (2), fraction of site occupied by water, side chain volume, hydropathy index, transfer free energy, CHP/water, hydrophobic parameter, distance between C-alpha and centroid of side chain, side chain angle theta(AAR), side chain torsion angle phi(AAAR), radius of gyration of side chain, van der Waals parameter R0, van der Waals parameter epsilon, normalized frequency of alpha-helix with weights, Normalized frequency of beta-sheet with weights, normalized frequency of reverse turn with weights, normalized frequency of alpha-helix (unweighted), normalized frequency of beta-sheet (unweighted), normalized frequency of reverse turn (unweighted), frequency of occurrence in beta-bends, conformational preference for all beta-strands, conformational preference for parallel beta-strands, conformational preference for antiparallel beta-strands, average surrounding hydrophobicity, normalized frequency of alpha-helix, normalized frequency of extended structure, normalized frequency of zeta R, normalized frequency of left-handed alpha-helix, normalized frequency of zeta L, normalized frequency of alpha region, refractivity, retention coefficient in HPLC (pH 7.4), retention coefficient in HPLC (pH 2.1), retention coefficient in NaClO4, retention coefficient in NaH2PO4, average reduced distance for C-alpha, average reduced distance for side chain, average side chain orientation angle, effective partition energy, normalized frequency of alpha-helix, normalized frequency of beta-structure, normalized frequency of coil, AA composition of total proteins, SD of AA composition of total proteins, AA composition of mt-proteins, normalized composition of mt-proteins, AA composition of mt-proteins from animal, normalized composition from animal, AA composition of mt-proteins from fungi and plant, normalized composition from fungi and plant, AA composition of membrane proteins, normalized composition of membrane proteins, transmembrane regions of non-mt-proteins, transmembrane regions of mt-proteins, ratio of average and computed composition, AA composition of CYT of single-spanning proteins, AA composition of CYT2 of single-spanning proteins, AA composition of EXT of single-spanning proteins, AA composition of EXT2 of single-spanning proteins, AA composition of MEM of single-spanning proteins, AA composition of CYT of multi-spanning proteins, AA composition of EXT of multi-spanning proteins, AA composition of MEM of multi-spanning proteins, 8 A contact number, 14 A contact number, transfer energy, organic solvent/water, average non-bonded energy per atom, short and medium range non-bonded energy per atom, long range non-bonded energy per atom, average non-bonded energy per residue, short and medium range non-bonded energy per residue, optimized beta-structure-coil equilibrium constant, optimized propensity to form reverse turn, optimized transfer energy parameter, optimized average non-bonded energy per atom, optimized side chain interaction parameter, normalized frequency of alpha-helix from LG, normalized frequency of alpha-helix from CF, normalized frequency of beta-sheet from LG, normalized frequency of beta-sheet from CF, normalized frequency of turn from LG, normalized frequency of turn from CF, normalized frequency of alpha-helix in all-alpha class, normalized frequency of alpha-helix in alpha+beta class, normalized frequency of alpha-helix in alpha/beta class, normalized frequency of beta-sheet in all-beta class, normalized frequency of beta-sheet in alpha+beta class, normalized frequency of beta-sheet in alpha/beta class, normalized frequency of turn in all-alpha class, normalized frequency of turn in all-beta class, normalized frequency of turn in alpha+beta class, normalized frequency of turn in alpha/beta class, HPLC parameter, partition coefficient, surrounding hydrophobicity in folded form, average gain in surrounding hydrophobicity, average gain ratio in surrounding hydrophobicity, surrounding hydrophobicity in alpha-helix, surrounding hydrophobicity in beta-sheet, surrounding hydrophobicity in turn, accessibility reduction ratio, average number of surrounding residues, intercept in regression analysis, slope in regression analysis ×1.0E1, correlation coefficient in regression analysis, hydrophobicity (2), relative frequency in alpha-helix, relative frequency in beta-sheet, relative frequency in reverse-turn, helix-coil equilibrium constant, beta-coil equilibrium constant, weights for alpha-helix at the window position of −6, weights for alpha-helix at the window position of −5, weights for alpha-helix at the window position of −4, weights for alpha-helix at the window position of −3, weights for alpha-helix at the window position of −2, weights for alpha-helix at the window position of −1, weights for alpha-helix at the window position of 0, weights for alpha-helix at the window position of 1, weights for alpha-helix at the window position of 2, weights for alpha-helix at the window position of 3, weights for alpha-helix at the window position of 4, weights for alpha-helix at the window position of 5, weights for alpha-helix at the window position of 6, weights for beta-sheet at the window position of −6, weights for beta-sheet at the window position of −5, weights for beta-sheet at the window position of −4, weights for beta-sheet at the window position of −3, weights for beta-sheet at the window position of −2, weights for beta-sheet at the window position of −1, weights for beta-sheet at the window position of 0, weights for beta-sheet at the window position of 1, weights for beta-sheet at the window position of 2, weights for beta-sheet at the window position of 3, weights for beta-sheet at the window position of 4, weights for beta-sheet at the window position of 5, weights for beta-sheet at the window position of 6, weights for coil at the window position of −6, weights for coil at the window position of −5, weights for coil at the window position of −4, weights for coil at the window position of −3, weights for coil at the window position of −2, weights for coil at the window position of −1, weights for coil at the window position of 0, weights for coil at the window position of 1, weights for coil at the window position of 2, weights for coil at the window position of 3, weights for coil at the window position of 4, weights for coil at the window position of 5, weights for coil at the window position of 6, average reduced distance for C-alpha, average reduced distance for side chain, side chain orientational preference, average relative fractional occurrence in A0(i), average relative fractional occurrence in AR(i), average relative fractional occurrence in AL(i), average relative fractional occurrence in EL(i), average relative fractional occurrence in E0(i), average relative fractional occurrence in ER(i), average relative fractional occurrence in A0(i−1), average relative fractional occurrence in AR(i−1), average relative fractional occurrence in AL(i−1), average relative fractional occurrence in EL(i−1), average relative fractional occurrence in E0(i−1), value of theta(i), value of theta(i−1), transfer free energy from chx to wat, transfer free energy from oct to wat, transfer free energy from vap to chx, transfer free energy from chx to oct, transfer free energy from vap to oct, accessible surface area, energy transfer from out to in (95% buried), mean polarity, relative preference value at N″, relative preference value at N′, relative preference value at N-cap, relative preference value at N1, relative preference value at N2, relative preference value at N3, relative preference value at N4, relative preference value at N5, relative preference value at Mid, relative preference value at C5, relative preference value at C4, relative preference value at C3, relative preference value at C2, relative preference value at C1, relative preference value at C-cap, relative preference value at C′, relative preference value at C″, Information measure for alpha-helix, information measure for N-terminal helix, Information measure for middle helix, information measure for C-terminal helix, information measure for extended, information measure for pleated-sheet, information measure for extended without H-bond, information measure for turn, information measure for N-terminal turn, information measure for middle turn, information measure for C-terminal turn, information measure for coil, information measure for loop, hydration free energy, mean area buried on transfer, mean fractional area loss, side chain hydropathy—uncorrected for solvation, side chain hydropathy—corrected for solvation, loss of side chain hydropathy by helix formation, transfer free energy, principal component I, principal component II, principal component III, principal component IV, Zimm-Bragg parameter s at 20 C, Zimm-Bragg parameter sigma ×1.0E4, optimal matching hydrophobicity, normalized frequency of alpha-helix, normalized frequency of isolated helix, normalized frequency of extended structure, normalized frequency of chain reversal R, normalized frequency of chain reversal S, normalized frequency of chain reversal D, normalized frequency of left-handed helix, normalized frequency of zeta R, normalized frequency of coil, normalized frequency of chain reversal, relative population of conformational state A, relative population of conformational state C, relative population of conformational state E, electron-ion interaction potential, bitterness, transfer free energy to lipophilic phase, average interactions per side chain atom, RF value in high salt chromatography, propensity to be buried inside, free energy change of epsilon(i) to epsilon(ex), free energy change of alpha(Ri) to alpha(Rh), free energy change of epsilon(i) to alpha(Rh), polar requirement, hydration potential, principal property value z1, principal property value z2, principal property value z3, unfolding Gibbs energy in water (pH 7.0), unfolding Gibbs energy in water (pH 9.0), activation Gibbs energy of unfolding (pH 7.0), activation Gibbs energy of unfolding (pH 9.0), dependence of partition coefficient on ionic strength, hydrophobicity (3), bulkiness, polarity, isoelectric point, RF rank, normalized positional residue frequency at helix termini N4′, normalized positional residue frequency at helix termini N′″, normalized positional residue frequency at helix termini N″, normalized positional residue frequency at helix termini N′, normalized positional residue frequency at helix termini Nc, normalized positional residue frequency at helix termini N1, normalized positional residue frequency at helix termini N2, normalized positional residue frequency at helix termini N3, normalized positional residue frequency at helix termini N4, normalized positional residue frequency at helix termini N5, normalized positional residue frequency at helix termini C5, normalized positional residue frequency at helix termini C4, normalized positional residue frequency at helix termini C3, normalized positional residue frequency at helix termini C2, normalized positional residue frequency at helix termini C1, normalized positional residue frequency at helix termini Cc, normalized positional residue frequency at helix termini C′, normalized positional residue frequency at helix termini C″, normalized positional residue frequency at helix termini C′″, normalized positional residue frequency at helix termini C4′, Delta G values for the peptides extrapolated to 0 M urea, helix formation parameters (delta G), normalized flexibility parameters (B-values)—average, normalized flexibility parameters (B-values) for each residue surrounded by none rigid neighbors, normalized flexibility parameters (B-values) for each residue surrounded by one rigid neighbors, normalized flexibility parameters, Free energy in alpha-helical conformation, free energy in alpha-helical region, Free energy in beta-strand conformation, free energy in beta-strand region, free energy in beta-strand region, free energies of transfer of AcW1-X-LL peptides from bilayer interface to water, thermodynamic beta sheet propensity, turn propensity scale for transmembrane helices, alpha helix propensity of position 44 in T4 lysozyme, p-Values of mesophilic proteins based on the distributions of B values, p-Values of thermophilic proteins based on the distributions of B values, distribution of amino acid residues in the 18 non-redundant families of thermophilic proteins, distribution of amino acid residues in the 18 non-redundant families of mesophilic proteins, distribution of amino acid residues in the alpha-helices in thermophilic proteins, distribution of amino acid residues in the alpha-helices in mesophilic proteins, side-chain contribution to protein stability (kJ/mol), propensity of amino acids within pi-helices, hydropathy scale based on self-information values in the two-state model (5% accessibility), hydropathy scale based on self-information values in the two-state model (9% accessibility), hydropathy scale based on self-information values in the two-state model (16% accessibility), hydropathy scale based on self-information values in the two-state model (20% accessibility), hydropathy scale based on self-information values in the two-state model (25% accessibility), hydropathy scale based on self-information values in the two-state model (36% accessibility), hydropathy scale based on self-information values in the two-state model (50% accessibility), averaged turn propensities in a transmembrane helix, alpha-helix propensity derived from designed sequences, beta-sheet propensity derived from designed sequences, composition of amino acids in extracellular proteins (percent), composition of amino acids in anchored proteins (percent), composition of amino acids in membrane proteins (percent), composition of amino acids in intracellular proteins (percent), composition of amino acids in nuclear proteins (percent), surface composition of amino acids in intracellular proteins of thermophiles (percent), surface composition of amino acids in intracellular proteins of mesophiles (percent), surface composition of amino acids in extracellular proteins of mesophiles (percent), surface composition of amino acids in nuclear proteins (percent), interior composition of amino acids in intracellular proteins of thermophiles (percent), interior composition of amino acids in intracellular proteins of mesophiles (percent), interior composition of amino acids in extracellular proteins of mesophiles (percent), interior composition of amino acids in nuclear proteins (percent), entire chain composition of amino acids in intracellular proteins of thermophiles (percent), entire chain composition of amino acids in intracellular proteins of mesophiles (percent), entire chain composition of amino acids in extracellular proteins of mesophiles (percent), entire chain composition of amino acids in nuclear proteins (percent), screening coefficients gamma (local), screening coefficients gamma (non-local), slopes tripeptide—FDPB VFF neutral, slopes tripeptides—LD VFF neutral, slopes tripeptide—FDPB VFF noside, slopes tripeptide FDPB VFF all, slope tripeptide FDPB PARSE neutral, slopes dekapeptide—FDPB VFF neutral, slopes proteins—FDPB VFF neutral, side-chain conformation by gaussian evolutionary method, amphiphilicity index, volumes including the crystallographic waters using the ProtOr, volumes not including the crystallographic waters using the ProtOr, electron-ion interaction potential values, hydrophobicity scales, hydrophobicity coefficient in RP-HPLC—C18 with 0.1% TFA/MeCN/H2O, hydrophobicity coefficient in RP-HPLC—C8 with 0.1% TFA/MeCN/H2O, hydrophobicity coefficient in RP-HPLC—C4 with 0.1% TFA/MeCN/H2O, hydrophobicity coefficient in RP-HPLC—C18 with 0.1% TFA/2-PrOH/MeCN/H2O, hydrophilicity scale, retention coefficient at pH 2, modified Kyte-Doolittle hydrophobicity scale, interactivity scale obtained from the contact matrix, interactivity scale obtained by maximizing the mean of correlation coefficient over single-domain globular proteins, interactivity scale obtained by maximizing the mean of correlation coefficient over pairs of sequences sharing the TIM barrel fold, linker propensity index, knowledge-based membrane-propensity scale from 1D_Helix in MPtopo databases, knowledge-based membrane-propensity scale from 3D_Helix in MPtopo databases, linker propensity from all dataset, linker propensity from 1-linker dataset, linker propensity from 2-linker dataset, linker propensity from 3-linker dataset, linker propensity from small dataset, linker propensity from medium dataset, linker propensity from long dataset, linker propensity from helical, linker propensity from non-helical (annotated by DSSP) dataset, stability scale from the knowledge-based atom-atom potential, relative stability scale extracted from mutation experiments, buriability, linker index, mean volumes of residues buried in protein interiors, average volumes of residues, hydrostatic pressure asymmetry index—PAL hydrophobicity index (2), average internal preferences, hydrophobicity-related index, apparent partition energies calculated from Wertz-Scheraga index, apparent partition energies calculated from Robson-Osguthorpe index, apparent partition energies calculated from Janin index, apparent partition energies calculated from Chothia index, hydropathies of amino acid side chains—neutral form, hydropathies of amino acid side chains—pi-values in pH 7.0, weights from the IFH scale, hydrophobicity index 3.0 pH, scaled side chain hydrophobicity values, hydrophobicity scale from native protein structures, NNEIG index, SWEIG index, PRIFT index, PRILS index, ALTFT index, ALTLS index, TOTFT index, TOTLS index, relative partition energies derived by the Bethe approximation, optimized relative partition energies—method A, optimized relative partition energies—method B, optimized relative partition energies—method C, optimized relative partition energies—method D, hydrophobicity index (3) and hydrophobicity index (4) and combinations thereof.

In a preferred embodiment, pre-defined features comprise any one or more of polar, non-polar, hydrophobic, helix/turn motif, β-sheet structure motif, charge of main chain, charge of side chain, solvent accessibility of an amino acid, spatial flexibility of the main chain and spatial flexibility of side chain of an amino acid.

In one preferred embodiment of the invention, the peptide variant(s) with a pre-defined length is 9 amino acid long and pre-defined features comprise any one or more of polar, non-polar, hydrophobic, helix/turn motif, β-sheet structure motif, charge of main chain, charge of side chain, solvent accessibility of an amino acid, spatial flexibility of the main chain and spatial flexibility of side chain of an amino acid. In one embodiment of the invention, the pre-defined features comprise hydrophobic and helix/turn motif.

In another preferred embodiment of the invention, the peptide variant(s) with a pre-defined length and pre-defined features comprise at least hydrophobic and helix/turn motif. For example, the peptide variant(s) with a pre-defined length may be 9 amino acids long and pre-defined features comprise hydrophobic and helix/turn motif.

In accordance with the practice of one aspect of the invention, the predictive ability of the peptide variant(s) to interact with the TCR comprises a numerical value or set of numerical values in which the value or set of numerical values is reflective of the degree of matching of the features associated with the amino acids of the peptide variant(s) to the pre-defined features for each position of the peptides recognized by TCR-associated with either CD8 + T-cell or CD4+ T-cell.

Further, obtaining the pre-defined features for each position of peptides recognized by TCR-associated with either CD8+ T-cell or CD4+ T-cell comprises a) aligning end-to-end peptides of same size with pre-defined length known to be bound by TCR-associated with either CD8+ T-cell or CD4+ T-cell; b) optionally, aligning end-to-end peptides of same size as in (a) known not to be bound by TCR-associated with either CD8+ T-cell or CD4+ T-cell but known to be bound by either MHC class I protein(s) or MHC class II protein(s); and c) determining amino acid features most prevalent or avoided at each amino acid position from the aligned sequences in (a) and/or (b); thereby, obtaining the pre-defined features for each position of peptides recognized by TCR-associated with either CD8+ T-cell or CD4+ T-cell.

In one embodiment of the invention, the selected peptide variant(s) with a predicted ability to interact with the TCR and may or can serve as a mammalian tumor vaccine(s) may be any of the peptides provided in Table 1.

In accordance with the practice of the invention, the methods of the invention may further comprise predicting a rank ordered list of the immunogenic peptides derived from mammalian tumor cell or mammalian tumor tissue so selected. The peptide may be a peptide variant. Moreover, rank ordering peptides may be based on a combination of the following parameters: a) expression of variant gene from which variant peptide is derived; b) predicted ability to bind TCR of CD8+ T-cell; c) binding affinity of the peptide to MHC class-I protein(s); d) peptide processing by proteases; and/or e) peptide transporter binding. Further, each parameter may be subdivided to reflect quality of the parameter through numerical value(s) or range(s) of values, and further, the numerical value(s) or range(s) of values from the parameters assessed or combined so as to produce output(s) permissive of sorting by ascending or descending order, thereby predicting a rank ordered list of the immunogenic peptides derived from mammalian tumor cell or mammalian tumor tissue so selected.

In another embodiment, the methods of the invention may further comprise predicting a rank ordered list of immunogenic peptides derived from mammalian tumor cell or mammalian tumor tissue, wherein the peptide is a peptide variant and wherein rank ordering peptides is based on a combination of the following parameters: a) expression of variant gene from which variant peptide is derived; b) predicted ability to bind TCR of CD4+ T-cell; c) binding affinity of the peptide to MHC class-II protein(s); d) peptide processing by lysosome and/or endosome; and/or e) fusion of the endosomal and/or lysosomal vesicles with Golgi-derived vesicles to permit loading of the immunogenic peptide onto MHC class II proteins.

In one embodiment of the invention, the immunogenic peptide so selected may be further selected by its ability to bind MHC class-I or class-II protein(s) or for its ability to bind a specific MHC class-I protein derived from a particular allele of MHC class I gene or specific MHC class-II proteins derived from two particular MHC class II genes. For example, the MHC class-I or class-II protein(s) may be encoded by the human leukocyte antigen gene complex (HLA). As a further example, the particular allele of MHC class I gene may be encoded by HLA-A locus, HLA-B locus, HLA-C locus, HLA-E locus, HLA-F locus or HLA-G locus. Further examples of the particular allele of MHC class I gene may be selected from the set as shown in Table 2.

Additionally, in one embodiment, the specific MHC class-II proteins may be derived from two particular MHC class II genes to form a heterodimer of an alpha chain and a beta chain. For example, the heterodimer may be any or HLA-DM, HLA-DO, HLA-DP, HLA-DQ and HLA-DR. IN another example, the alpha chain of HLA-DM heterodimer may be encoded by HLA-DMA locus, alpha chain of HLA-DO heterodimer is encoded by HLA-DOA locus, alpha chain of HLA-DP heterodimer is encoded by HLA-DPA1 locus, alpha chain of HLA-DQ heterodimer is encoded by HLA-DQA1 locus or HLA-DQA2 locus, and alpha chain of HLA-DR is encoded by HLA-DR locus. In a further example, the beta chain of HLA-DM heterodimer may be encoded by any of HLA-DMB locus, beta chain of HLA-DO heterodimer is encoded by HLA-DOB locus, beta chain of HLA-DP heterodimer is encoded by HLA-DPB1 locus, beta chain of HLA-DQ heterodimer is encoded by HLA-DBQ1 locus or HLA-DQB2 locus, and beta chain of HLA-DR is encoded by HLA-DRB1 locus, HLA-DRB3 locus, HLA-DRB4 or HLA-DRB5 locus. Further examples of the particular allele of MHC class II gene may be selected from the set as shown in Table 3.

In accordance with the invention the allele may be described by a classification system comprising HLA prefix, separated by hyphen, followed by HLA gene, field separator, serotype, protein coded by allele in order of discovery, one or more numbers designated by gene sequencing and expression, or a combination thereof. Currently, there are more than 7,670 MHC class I alleles and more than 2,260 MHC class II alleles. In addition, each locus may comprise multiple genes or alleles of MHC class-I or class-II protein(s).

In accordance with the invention, the methods of the invention may further comprise MHC-typing of the tumor cell or tumor tissue in selection of immunogenic peptide(s), so as to select immunogenic peptide(s) which may be displayed by the tumor cell or tumor tissue, by cells of individual or subject from which tumor cell or tumor tissue is derived, or by immune cells of individual or subject from which tumor cell or tumor tissue is derived.

In accordance with the invention, the methods of the invention may further comprise HLA-typing of the tumor cell or tumor tissue in selection of immunogenic peptide(s), so as to select immunogenic peptide(s) which may be displayed by the tumor cell or tumor tissue, by cells of individual or subject from which tumor cell or tumor tissue is derived, or by immune cells of individual or subject from which tumor cell or tumor tissue is derived.

In one embodiment of the invention, the prediction of immunogenic peptide(s) may further comprise MHC-typing analysis comprising the steps of: a) determining serotype or expressed isotype or supertype of MHC class-I or class-II protein(s) expressed by MHC class-I or class-II genes of the mammalian tumor cell or tumor tissue, or alternatively of the cell or immune cell of an individual or subject to be administered with mammalian tumor vaccine(s) comprising the predicted immunogenic peptide(s); b) calculating probability of MHC class-I or class-II protein(s) of (a) binding mammalian tumor peptide variant(s) with optimal processing sites from a library of tumor peptide variants; c) calculating probability of TCR binding to generate a T-cell response; d) selecting tumor peptide variant(s) having highest probability from steps (b) that can modulate the immune response of a mammal when challenged with the tumor peptide variant(s), thereby further selecting mammalian tumor vaccine(s) dependent on MHC class-I or class-II expression of the mammalian tumor cell or tumor tissue, or alternatively of the cell or immune cell of an individual or subject to be administered with mammalian tumor vaccine(s) comprising the predicted immunogenic peptide(s).

In another embodiment, the prediction of immunogenic peptide(s) may further comprise the steps of HLA-typing analysis comprising: a) determining serotype or expressed isotype or supertype of HLA protein(s) expressed by HLA genes of the mammalian tumor cell or tumor tissue, or alternatively of the cell or immune cell of an individual or subject to be administered with mammalian tumor vaccine(s) comprising the predicted immunogenic peptide(s); b) calculating probability of HLA protein(s) of (a) binding mammalian tumor peptide variant(s) with optimal processing sites from a library of tumor peptide variants; c) calculating probability of TCR binding to generate a T-cell response; d) selecting tumor peptide variant(s) having highest probability from steps (b) that can modulate the immune response of a mammal when challenged with the tumor peptide variant(s), thereby further selecting mammalian tumor vaccine(s) dependent on HLA expression of the mammalian tumor cell or tumor tissue, or alternatively of the cell or immune cell of an individual or subject to be administered with mammalian tumor vaccine(s) comprising the predicted immunogenic peptide(s).

In accordance with the invention, the mammalian tumor vaccine(s) may comprise the selected immunogenic peptide so identified by computation method.

Further, in accordance with the invention, selected immunogenic peptide in the mammalian tumor vaccine(s) may have amino-terminal and carboxyl-terminal extensions. For example, the amino-terminal and carboxyl-terminal extensions may be amino acids. The amino acids in the amino-terminal and carboxyl-terminal extensions may permit processing of the selected immunogenic peptide of claim 1 or 3 so as to be displayed by the MHC class I protein(s) and/or the MHC class II protein(s). For example, the MHC class I protein(s) and/or the MHC class II proteins(s) may be associated with a human. Further, the MHC class I protein(s) and/or the MHC class II protein(s) associated with a human may be an HLA protein(s).

Additionally, the invention provides methods of preparing a subject-specific immunogenic peptide composition comprising selecting cancer vaccines from genetically altered proteins expressed by mammalian cancer cells and tissues by any of the methods of the invention. Merely by way of example, said subject-specific peptides, may comprise: (a) a peptide that has a non-synonymous mutation leading to different amino acids in comparison with a protein of the non-tumor sample; (b) a peptide having a read-through mutation in which a stop codon is modified or deleted, leading to translation of a longer protein in comparison with a protein of the non-tumor sample with a novel tumor-specific sequence at the C-terminus; (c) a peptide that has a splice site mutation that leads to the inclusion of an intron or part of an intron, or alternatively exclusion of an exon or part of an exon, in the mature mRNA and thus has a unique tumor-specific protein sequence; (d) a peptide representing a chromosomal rearrangement that has given rise to a chimeric protein with tumor-specific sequences at the junction of two proteins of the non-tumor sample and thus represents a gene fusion; or (e) a peptide representing in comparison with a protein of the non-tumor sample a frameshift mutation or deletion that leads to a new open reading frame and a novel tumor-specific protein sequence. The subject-specific immunogenic composition may comprise a subject-specific peptide that binds to the HLA protein of the subject with an IC50 less than about 500 nM.

The invention additionally provides methods of treating a subject having cancer. In one embodiment, the method comprises administering in the subject an immunogenic peptide, composition of the invention or cancer vaccines so selected by any of the methods of the invention in a sufficient amount so as to treat the cancer.

In another embodiment, the method comprises a) obtaining a sample from the subject; b) identifying the genetically altered protein(s) expressed by the mammalian tumor cell or the mammalian tumor tissue in the sample through nucleic acid sequence(s) encoding the altered protein(s); b) producing peptide fragment(s) comprising at least one amino acid mutation from the genetically altered protein(s) so identified in step (a), so as to obtain peptide variant(s) associated with the mammalian tumor cell or the mammalian tumor tissue. Then the method further comprises selecting the peptide variant(s) from step b, which binds a T-cell receptor (TCR). This step comprises: i) selecting the peptide variant(s) with a pre-defined length; ii) characterizing the peptide variant(s) (e.g. in silico) by selecting and matching features associated with an amino acid at each position of the peptide with selected pre-defined features for each position of peptides recognized by TCR associated with either CD8+ T-cell or CD4+ T-cell, so as to obtain predictive ability of the peptide variant(s) to interact with the TCR; iii) selecting the peptide variant(s) above based on predicted ability of the peptide variant(s) to interact with the TCR, so as to be an immunogenic peptide that may or can serve as a mammalian tumor vaccine(s) after lengthening the selected immunogenic peptide variant(s) such that following vaccination the lengthened selected peptide variant(s) is taken up by antigen-presenting cells, processed to the size of the selected peptide variant(s) and displayed by antigen-presenting cells. The method further comprises forming a vaccine comprising the at least one immunogenic peptide so selected and administering the vaccine in an effective amount to the subject so as to treat the cancer in the subject.

For example, the cancer may be a stomach cancer, a colon cancer, a breast cancer, an ovarian cancer, a prostate cancer, a lung cancer, a kidney cancer, a gastric cancer, a testicular cancer, a head and neck cancer, a pancreatic cancer, a brain cancer, a melanoma, a lymphoma or a leukemia.

Immunogenic Peptides from Mutated or Altered Proteins in Mammalian Cancers

The invention further provides an immunogenic peptide composition prepared by this method of the invention. In one embodiment, the immunogenic peptide composition may further comprise at least one adjuvant.

The invention further provides a mammalian tumor vaccine selected by any of the methods of the invention.

The methods described herein in various embodiments comprise identifying immunogenic peptides of nine amino acids (9-mer) derived from mutations present in mammalian cancer tissues and cancer cell lines. In the context of this disclosure, immunogenic peptides are selected on the basis of: i) TCR binding; ii) HLA binding; iii) expression; iv) proteolytic processing; and v) peptide transporter binding. The method described in various embodiments was applied to 2.3 million unique cancer mutations captured from MedGenome's proprietary cancer mutation database OncoMD™ and a list of peptides restricted to class I HLA molecules consisting of HLA-A01:01, HLA-A02:0, HLA-A11:01, HLA-A24:02, HLA-B35:03, HLA-B40:06, HLA-B44:03. HLA-B51:01, HLA-B57:01, HLA-C06:02, HLA-C07:02, HLA-C12:03, HLA-C15:02 are identified (Table 1). In some embodiments, one or more of the 9-mer immunogenic peptide identified by the methods of the invention can be used following amino acid extension (addition) on amino-terminus and carboxyl-terminus, as a cancer vaccine and administered to cancer patients. In an embodiment, equal number of amino acids are added at each end of the 9-mer peptide identified by the methods of the invention, so as to permit cross presentation of the desired 9-mer immunogenic peptide. In some embodiments, the composition of a cancer vaccine may comprise of two or more immunogenic peptides. In some embodiments, cancer vaccines comprising of one, two or more immunogenic peptides may activate a cytotoxic T cell (CTL) response and a CD4 T cell response against one or two or more immunogenic peptides.

In some embodiments, the cancer vaccine composition may comprise of a 9-mer immunogenic peptide that may be part of a precursor protein, or part of longer peptides about >9 amino acids up to about 50 amino acids. In some embodiments, the cancer vaccine composition may comprise of two or more immunogenic peptides that may be part of one, two or more precursor proteins or part of one, two or more longer peptides about >9 amino acids up to about 50 amino acids. In some embodiments, the composition of the cancer vaccine may contain an adjuvant to help boost the immune response. In some embodiments, the composition of the cancer vaccine containing an adjuvant to help boost the immune response may be pharmaceutically acceptable.

In some embodiments, the cancer vaccine, or a precursor protein containing the cancer vaccine, or a longer peptide about >9 amino acids up to about 50 amino acids containing the cancer vaccine may be encoded by a nucleic acid sequence. In some embodiments, the nucleic acid sequence may be a DNA. In other embodiments, the nucleic acid sequence may be RNA. In some embodiments, the nucleic acid sequence may contain an adjuvant. In some embodiments, the nucleic acid sequence with the adjuvant may be used for treating the cancer patients.

In some embodiments, the nucleic acid sequence may be injected into mammalian cells to express the cancer vaccine in the form of a peptide, or as part of a protein precursor or as part of a longer peptide >9 amino acid up to about 50 amino acids to generate stable cells. In some embodiments, the stable cells may be primary cells, or cell lines derived from primary cells. In some embodiments, the primary cell may be derived from normal tissues or from cancer tissues.

In some embodiments, the stable cells may be used for screening antibodies by phage display technology. In some embodiments, the stable cells may be used in T cell activation screening assays.

### Combination Therapy

In another embodiment, the peptides of the invention (e.g., single or multiple peptides of the invention) so obtained by the methods of selection of the invention may be administered in combination, or sequentially, with another therapeutic agent. Such other therapeutic agents include those known for treatment, prevention, or amelioration of one or more symptoms of cancer diseases and disorders. Such therapeutic agents include, but are not limited to, ricin. ricin A-chain, doxorubicin, daunorubicin, taxol, ethiduim bromide, mitomycin, etoposide, tenoposide, vincristine, vinblastine, colchicine, dihydroxy anthracin dione, actinomycin D, diphteria toxin, Pseudomonas exotoxin (PE) A, PE40, abrin, arbrin A chain, modeccin A chain, alpha-sarcin, gelonin, mitogellin, retstrictocin, phenomycin, enomycin, curicin, crotin, calicheamicin, sapaonaria officinalis inhibitor, maytansinoids, and glucocorticoid and other chemotherapeutic agents, as well as radioisotopes such as ²¹²Bi, ¹³¹I, ¹³¹In, ⁹⁰Y, and ¹⁸⁶Re.

The peptides of the invention formulated into tumor or cancer vaccine(s) may also be used in combination, or sequentially, with one or more immune checkpoint inhibitors. Immune checkpoint inhibitors include inhibitors for PD-1, PD-L1, PD-L2, 4-1BB, 4-1BBL, HVEM, BTLA, CD160, CD226, LAG3, CTLA-4, B7-1, B7-2, CD40, CD40L, Galectin-9, TIM-3, GITR, GITRL, SIRP alpha, B7-H3, B7-H4, VISTA, OX40, OX-40L, CEACAM1, CD47, ICOS, ICOSL, TIGIT, IDO, CD28, LIGHT, TIGIT, CD155, CD70 and adenosine A2a receptor. Immune checkpoint inhibitor may be an antibody or an antibody fragment. The antibody or antibody fragment may be derived from a monoclonal antibody. In one embodiment, the monoclonal antibody or its fragment is human or humanized. Immune checkpoint inhibitor for PD-1 may be selected from any of MEDI0680 (also known as AMP-614; MedImmune/AstraZeneca), nivolumab (also known as Opdivo, BMS-936558, MDX-1106 and ONO-4538; Bristol-Myers Squibb and Ono Pharmaceuticals), pembrolizumab (also known as Keytruda, MK-3475 and lambrolizumab; Merck) and pidilizumab (also known as CT-011; CureTech). Immune checkpoint inhibitor for PD-L1 may be selected from any of BMS-936559 (also known as CT-011; Bristol-Myers Squibb), MEDI4736 (MedImmune/AstraZeneca), MPDL3280A (also known as RG7446; Genetech/Roche) and MSB0010718C (EMD Serono).

### Kits

According to another aspect of the invention, kits are provided. Kits according to the invention include package(s) comprising antibodies or compositions of the invention.

The phrase “package” means any vessel containing peptides or compositions presented herein. In preferred embodiments, the package can be a box or wrapping. Packaging materials for use in packaging pharmaceutical products are well known to those of skill in the art. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment.

The kit can also contain items that are not contained within the package but are attached to the outside of the package, for example, pipettes.

Kits may optionally contain instructions for administering peptides or compositions of the present invention to a subject having a condition in need of treatment. Kits may also comprise instructions for approved uses of compounds herein by regulatory agencies, such as the United States Food and Drug Administration. Kits may optionally contain labeling or product inserts for the present compounds. The package(s) and/or any product insert(s) may themselves be approved by regulatory agencies. The kits can include antibodies in a solid phase or in a liquid phase (such as buffers provided) in a package. The kits also can include buffers for preparing solutions for conducting the methods, and pipettes for transferring liquids from one container to another.

The kit may optionally also contain one or more other agents for use in combination therapies as described herein. In certain embodiments, the package(s) is a container for intravenous administration. In other embodiments antibodies are provided in the form of a liposome.

The following examples serve to illustrate the present invention. These examples are in no way intended to limit the scope of the invention.

### EXAMPLES

### Example 1

Selecting Immunogenic Peptide from Variant Coding Sequence

This application provides a method to combine protein sequence-altering variant identification with methods to predict immunogenic peptides from mutated proteins. For example, in some embodiments the method provides immunogenic peptides from cancer tissues of an individual, where the individual can be mice or human.

Selection of immunogenic peptides comprises: a) selecting a set of cancer variants from mouse and human cancer cell lines and mouse and human cancer tissues where each variant in the genomic sequence correspond to both protein coding and protein non-coding sequences; b) variants of mouse cell lines and cancer tissues are identified by mouse whole exome and/or whole genome sequencing and variants from human cancer cell lines and human cancer tissues are identified by whole exome and/or whole genome sequencing; c) variants in mouse tissues and cell lines are identified by comparing with the reference sequence of mouse, and variants in human tissues and cell lines are identified by comparing with the reference sequence of human; d) variants are identified by comparing with the reference sequence, where the reference sequence is mouse reference sequence available in the public domain, or human reference sequence available in the public domain (e.g. current mouse reference sequence is (GRCm38/mm 10) and current human reference sequence is (hg19)); e) variants from mouse tissues and cell lines include all genomic variants that alter the sequence of the RNA and the sequence of the protein translated from the RNA; f) variants from human tissues and cell lines include all genomic variants that alter the sequence of the proteins translated from the messenger RNA-protein variants; g) selecting the variants based on their expression in the mouse or human cell lines and tissues from the transcriptomic analysis; h) generating 8-11 amino acid peptides from the altered protein variants; and/or i) selecting a set of 8-11 amino acid immunogenic peptides from the previous step by predicting immunogenicity of the variant peptide comprising the altered amino acids encoded by the variant coding sequence; thereby selecting immunogenic peptides from altered or mutated proteins unique to cancer or tumor cells or tissues.

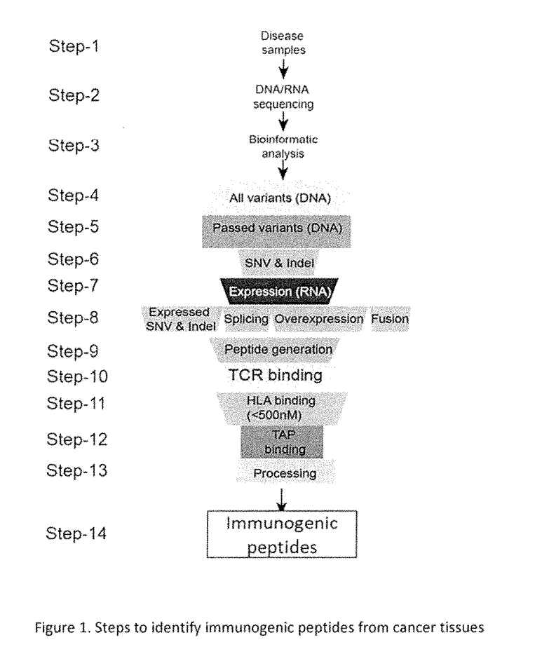

In some embodiments, cancer-specific mutant proteins are detected by sequencing DNA and RNA of all protein-coding genes encoded in mouse or human genome. In one embodiment, all protein coding genes are identified by whole exome sequencing (WES) or whole genome sequencing (WGS) The sequences are analyzed and taken through a series of steps shown in FIG. 1.

Brief description of the steps shown in FIG. 1 include the following.

Step 1 & 2 involve the use of MedGenome's next generation sequencing pipeline to identify genetic alterations at the DNA and RNA level.

Step 3 involves standard bioinformatic processing of next generation sequencing data to identify cancer-specific genetic alterations at the DNA and RNA level

Steps 4-6 use MedGenome's variant calling pipeline to identify all variants and select those that pass the quality control metrics (Passed variants). Passed variant is identified based on:

1. Alignment

2. Read depth

3. Allele depth,

4. Overall quality of the variant.

Sequence variants can generate different classes of altered proteins: i. proteins altered in amino acid sequence in which one or more amino acids are altered, which may be arranged in a sequence or distributed randomly across the length of the protein; ii. proteins translated from fusion genes; iii. proteins produced from splice variants and from mutations in splicing sites, which results in the introduction of intronic region, or part of an intronic region, or alternatively, exclusion of an exon or part of an exon, in frame with the protein coding sequence; iv. Proteins produced from insertions and deletions of nucleotides that cause frameshift in the protein coding sequence resulting in the introduction of one or more amino acids absent in the normal protein; v. Protein arising from loss of stop codons (stop loss) that adds additional amino acids at the end of the protein. In some embodiments, tumor or cancer tissues from individuals comprise more than 1, 100, 1000, 2,000, or 6,000 different variant coding sequences resulting in changes in amino acid(s) in the protein as compared to the reference sample.

Step 7 applies further selection by considering variants that are expressed in the cancer tissue using the transcript data from RNA sequencing. The RNA sequence data is analyzed using MedGenome's RNA analysis pipeline to identify expressed variants, identify splice variants, frameshift variants and fusion genes. The pipeline defines expression as ≥1 FPKM (1 fragment per kilobase per million).

Step 8 compiles a list of all the expressed variants that will result in the generation of altered proteins. These altered proteins are likely to be absent in normal tissues and are cancer specific. A variant is considered expressed if it has a value ≥1 FPKM. Fusion genes are identified when regions from two different genes are fused to each other, and are present as part of a transcript. The fusion gene is considered expressed if the fusion region has a value ≥1 FPKM

Step 9 generates peptides used in in silico TCR-binding analysis. Binding of TCRs to peptides occur when peptides are in complex with class-I or class-II HLA molecules. Class I HLA binds 8-11-mer peptides and Class II HLA binds 13-21 mer peptides. Our algorithm generates two sets of peptides for each mutation, one containing the non-mutated (wild-type) amino acid and the other corresponding to the mutant amino acid. The length of the peptide can vary from 8-mer to 21-mer. The algorithm automatically generates two sets of peptide libraries in which the wild-type or the mutant amino acid occupy each of the positions across the length of the peptide. For example, if a peptide is 9-mer long, the algorithm generates 9 wild-type peptides and 9 mutant peptides for in silico binding analysis by moving the mutant amino acid to each of the 9 positions in the peptide by a sliding window method.

Step 10 uses a novel algorithm that we have developed to identify immunogenic peptides that have a higher likelihood of eliciting a T-cell response. Peptides interact with TCR only if they are bound to the HLA molecule. The TCR interaction depends on the conformation of the peptide, the availability of amino acids that make contacts with the residues on the TCR, and the type of interactions that are made between residues on the peptide and the residues on the TCR. Our new method integrates information from sequence and structure of the peptides to model the TCR interaction and has been tested on gold standard datasets. The method may be computational or in silico.

Step 11 determines the binding affinity of both the wild-type and the mutant peptides with Class I or Class II HLA molecules. Mutant peptides with lower binding score are generally consider as strong binder to HLA molecule. After binding prediction, three groups of peptides are selected:

1. High affinity binding peptides—≤500 nM

2. Medium affinity binding peptides—>500 nM-≤1000 nM

3. Low affinity binding peptides—>1000 nM peptides

Step 12 screens peptides for optimal processing to identify proteasomal and/or immunoproteasomal processing sites around the peptide, with the objective of prioritizing peptides in which the processing sites are optimally located, such that upon processing, the correct size peptide is produced. This step is important because the class I and class II HLA molecules bind peptides of a particular length. Class I HLA binds peptides from 8-11 mer and Class II HLA binds peptides that are 13-21 mer. We have devised our own scoring method that takes into account the presence of processing sites at the N and C-terminal ends of the peptide. When both sites are optimally located a maximum score of 20 is given. The score decreases as the processing sites are shifted away from the optimal location. A score >10 is used to select peptides for the next step. Peptides that are scored higher than 10 either by the proteasomal or by the immunoproteasomal cleavage are selected.

Step 14 calculates the transporter (TAP) binding affinity of the peptides. In order for the peptide to bind HLA molecule, the peptide needs to be transported from cytosol to endoplasmic reticulum. In this step, we perform the analysis to identify whether the peptide is delivered to HLA molecule by TAP. Any peptide exhibiting a TAP-binding score of <0.5 are selected for the final step of prioritization.

Predicting Immunogenic Peptides by their Ability to Bind TCRs

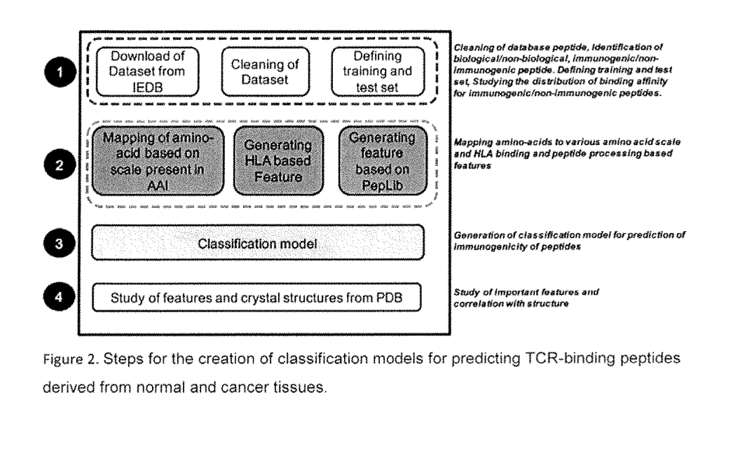

The prediction of TCR-binding peptide prediction involves four different steps: 1. Data set creation; 2. Feature creation; 3. Classification model; 4. Study of features. The steps are shown in FIG. 2. A brief description of each step:

1. Dataset creation: In this step, we have first collected peptide and its immunogenicity status from IEDB database. After this we then performed processing of the peptides to have a clean dataset for the model building exercise. Further, we have generated several training and test instances for model building and performance evaluation.

2. Feature creation: In this step, various amino acid features, HLA binding and peptide processing related feature is generated for the peptides.

3. Classification model: In this step, classification model is generated using feature matrix. This step involves: feature selection, identification of classification method, scoring of the peptides.

4. Study of features: The important features are studied in detail and its correlation with peptide structure/interactions in crystal structure is also studied in this step.

Data Preparation

The sequence, assay, HLA type, publication id (PMID), and immunogenicity information of the peptide was downloaded from IEDB database (Release 24 Nov. 2016). The database contains immunogenicity status for 2,521 unique 9-mer peptides for human. The peptide is first categorized into self and foreign peptide. The peptides generated by human body are known as self, while those that do not originate in human body are called non-self or foreign peptides. Of the total peptides, ˜85% of them belong to foreign peptide category. The peptides are also classified based on assay that was performed to check its immunogenicity. Although there are several assay types, we have broadly grouped them into biological and non-biological type. Majority of the peptides (˜90%) are assayed by biological type. Before using these peptides, we apply the following filters to focus on unambiguous assay prediction and for which the information as per our requirement is complete.
Biological assay filter: The peptides predicted as immunogenic/non-immunogenic using one of the biological assay is taken further for the analysis.
Prediction by assays: There are many peptides which are predicted as both immunogenic and non-immunogenic using one or more different assays. These peptides were removed from our analysis.
4-digit HLA information: The peptides for which 4-digit information is available for the HLA type is considered for further analysis. Of the total peptides, for 1075 peptides 4-digit HLA information was available

Overall, we obtain 1,075 peptides for which unambiguous immunogenicity and HLA 4-digit information is complete. The classification model was built using 307 immunogenic peptides (Table 8) and 167 non-immunogenic peptides (Table 9). These peptides bind HLA-A02:01.

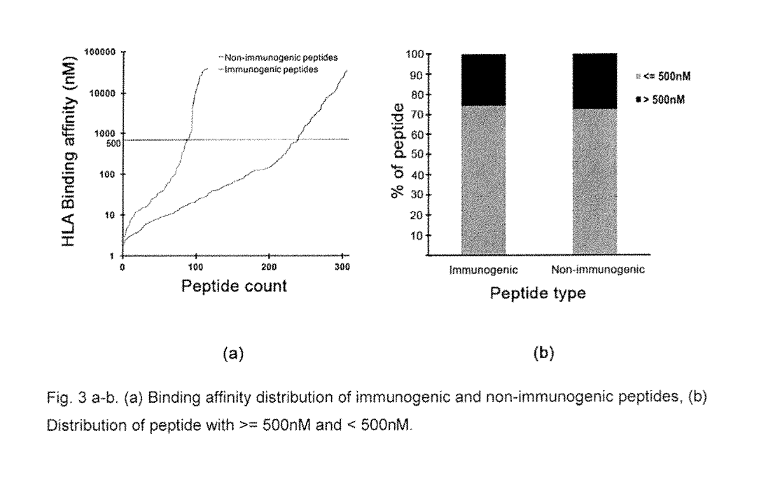

Currently the binding affinity of the peptide is considered as the main criteria to select immunogenic peptides. In general, binding affinity by standard programs such as NetMHCcons [24] with <=500 nM is taken as cutoff to define immunogenic peptides. The distribution of binding affinity for the HLA-A*02:01 peptides is shown in FIG. 3. If we consider <=500 nM as cutoff to define immunogenic peptides then the sensitivity is 74.5% whereas the specificity is only 27.6%. FIG. 3B demonstrates that HLA binding does not predict immunogenic peptides because both non-immunogenic and immunogenic peptides can bind HLA with high affinity (FIG. 3B).

Feature Construction and Selection

In order to generate features that will discriminate the TCR-binding peptides from the non-binders, we analyzed the physico-chemical composition of the amino acids and their positional biases in the 9-mer peptides that interact with TCR when bound to the HLA molecule. We analyzed 58 crystal structure data of TCR-HLA-peptide complex to identify binding interactions that existed at each position of the 9-mer peptide and the HLA at one hand and the TCR on the other. A summary of the feature types is provided below:

I. Physicochemical features: An amino acid is an organic molecule with an amino group (—NH2) and a carboxyl group (—COOH). We obtained the physicochemical features from following two different sources.
AAindex: AAindex is a database that contains numerical representation for various physicochemical and biochemical properties of amino acids and pairs of amino acids. We used AAindex1 for our feature creation. Most of the defined indices belong to 4 major cluster—(i) α-helix and turn propensities, (ii) β-strand propensity, (iii) hydrophobicity and (v) physicochemical properties. A total of 566 different AAindex1 scale was obtained from this database (May 18, 2017). We use the following strategy to generate features.
   AAIF₁: The value of AAindex1 scale for peptide position #1.
   AAIF₂: The value of AAindex1 scale for peptide position #2.
   AAIF₃: The value of AAindex1 scale for peptide position #3.
   AAIF₄: The value of AAindex1 scale for peptide position #4.
   AAIF₅: The value of AAindex1 scale for peptide position #5.
   AAIF₆: The value of AAindex1 scale for peptide position #6.
   AAIF₇: The value of AAindex1 scale for peptide position #7.
   AAIF₈: The value of AAindex1 scale for peptide position #8.
   AAIF₉: The value of AAindex1 scale for peptide position #9.
   AAIF₁₋₂: The average value of AAindex1 scale for peptide position #1 and #2.
   AAIF₂₋₃: The average value of AAindex1 scale for peptide position #2 and #3.
   AAIF₃₋₄: The average value of AAindex1 scale for peptide position #3 and #4.
   AAIF₄₋₅: The average value of AAindex1 scale for peptide position #4 and #5.
   AAIF₅₋₆: The average value of AAindex1 scale for peptide position #5 and #6.
   AAIF₆₋₇: The average value of AAindex1 scale for peptide position #6 and #7.
   AAIF₇₋₈: The average value of AAindex1 scale for peptide position #7 and #8.
   AAIF₈₋₉: The average value of AAindex1 scale for peptide position #8 and #9.
   AAIF₃₋₈: The average value of AAindex1 scale from peptide position #3 to position #8.
   AAIF₁₋₉: The average value of AAindex1 scale from peptide position #1 to position #9.

Overall, we generated 11,300 features from AAindex.
PepLib: Peplib is a R package that can be used to calculate the descriptors for each amino acid of given peptide sequence. These descriptors include counts of groups (polar, acidic, basic, aromatic etc.), molecular weight, number of rotatable bonds and charged based partial surface area descriptors. There are 53 variables to be calculated for each amino acid in the peptide sequence. Some of these descriptors are based on permutation of descriptors calculated on single amino acid. Along with the descriptors calculated for each amino acid. Peplib provides the values at sequence level also. Sequence level calculation involves three types of the descriptors—1. mean 2. variance and 3. autocorrelation function of the descriptors for each sequence.

II. HLA binding feature: Prediction of HLA binding affinity score is the most important feature of the peptide that is being currently used by community to identify candidate T cell epitopes. Binding affinity of <=500 nM is routinely used as a threshold for peptide selection. We have generated NetMHCcons binding affinity score as one of the feature for each peptide. NetMHCcons is a consensus based method of three different state-of-the-art MHC-peptide binding prediction methods (NetMHC, NetMHCpan and PickPocket) with peptides. NetMHCcons uses artificial neural network-based method give result as IC50 values trained on data from various MHC alleles and positional specific scoring matrices [24].

III. Peptide processing features:
NetChop: Peptide cleavage is an important step for making sure that the peptide is generated for the transportation and then presentation by HLA molecule. We have used the IEDB NetChop 3.1 program [25] to identify the cleavage sites. NetChop is a neural network prediction based method for prediction of cleavage sires of the human proteasome. We generate two different features for each peptide—(a) C-term which is trained with the database consisting of publicly available MHC class I ligands using C-terminal cleavage sites of ligand into consideration, (b) 20s which is trained with the in vitro degradation data.
TAP processing: The TAP processing includes the neural network based estimation of ability of transportation of cleaved peptides by TAP transporter proteins to the endoplasmic reticulum. The neural network is trained on the in vitro experiments characterizing the sequence specificity of TAP transport. In total, six features based on TAP were generated for each of the peptides.

Overall, from the total peptides 307 immunogenic and 116 non-immunogenic peptides that bind HLA-A*02:01, we generated 12,094 total features.

Classification Model

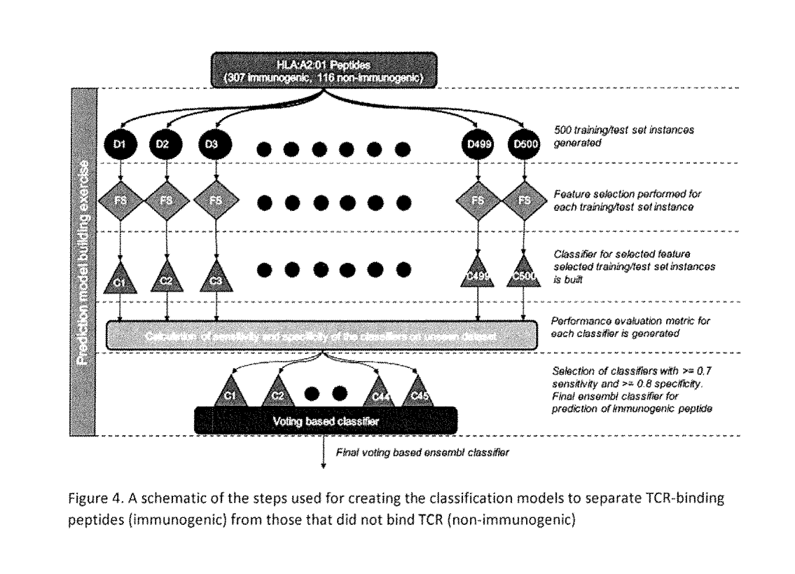

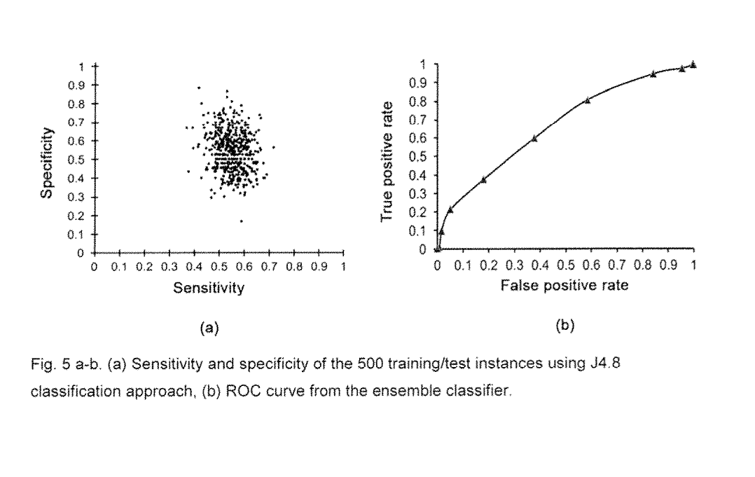

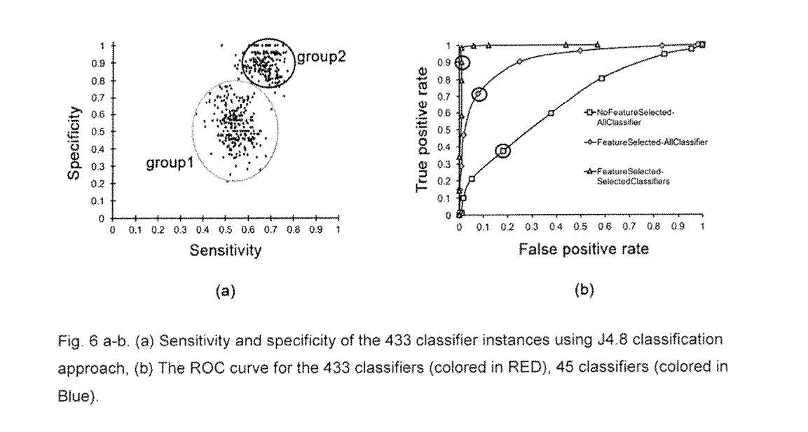

We performed the following steps to generate the classification model for predicting immunogenicity of the peptides as shown in FIG. 4.
Creation of training and test set instances: Due to unbalanced dataset of immunogenic and non-immunogenic peptides (3:1) in our study, we first generated 500 different instances of the complete dataset which had balanced number of immunogenic and non-immunogenic peptides. Each balanced dataset consists of ˜100 immunogenic and non-immunogenic peptides. The balance dataset is generated to avoid overfitting of classification model to either immunogenic or non-immunogenic peptide class.
Feature selection: We generated classification model using all 12,094 features for 500 training/test instances. Ensemble classifier is generated by combining the results from all classifier instances. Equal weight is given to each of the classifier instance. If >50% of classifier predict a peptide as immunogenic then the prediction of the ensemble classifier is taken as immunogenic otherwise prediction is taken as non-immunogenic. The sensitivity and specificity of J4.8 classifier for the 500 instances is shown in FIG. 5A. The ROC curve of the ensemble classifier is shown in FIG. 5B. The ROC curve is generated by changing the cutoff/threshold of ensemble classifier for predicting a peptide as immunogenic or non-immunogenic.
Feature reduction: As a next step, we performed feature reduction for each 500 instances using CfsSubsetEval method available in Weka machine learning toolkit [26]. This method evaluates the worth of a subset of attributes by considering the individual predictive ability of each feature along with the degree of redundancy between them. During feature selection, some of the training instance failed to converge, hence, we were left with 433 training instances. A median of 45 features were selected for each training instance. Overall, 3680 features were selected when all 433 training instances were included. Of these 60% (2219) of the features were part of 2 or more training instances. Using the reduced 433 training instances a new classification model was built.
Performance evaluation of classifier instances: The reduced features for each training instances was trained using J4.8 classification system. We first created an ensemble classifier by combining the prediction from all 433 classifier instances. A sensitivity/specificity plot using 3680 features clearly separates the classifier instances into two groups (FIG. 6A). The Group-2 classifier instances have higher sensitivity and specificity as compared to Group-1 classifier instances (FIG. 6A). We used voting based approach to classify the peptide sequence into immunogenic and non-immunogenic class. For an input peptide if >50% of the classifiers predicts it as immunogenic then the peptide is classified as immunogenic otherwise the peptide is defined as non-immunogenic peptide. ROC curve of 433 classifier instances (Ensemble classifier2) performs better than using 500 classifier instances (Ensemble classifier1) (FIG. 6B).
In the next step, we selected classifier instances for which >=75% sensitivity and >=80% specificity on unseen dataset was observed. We found 45 such classifier instances. An ensemble classifier was created using the 45 classifiers. ROC curve of 45 classifier instances (Ensemble classifier3) is shown in FIG. 6B.

Performance evaluation of the three ensemble classifiers on unseen dataset is shown in Table 10. Ensemble3 classifier provides sensitivity and specificity of 90.23% and 99.14% respectively, which is significantly higher than the HLA binding affinity of the peptides. Table 10 demonstrates that the HLA binding affinity, which is currently used as an important criterion for selecting immunogenic peptides carry a high false positive rate.

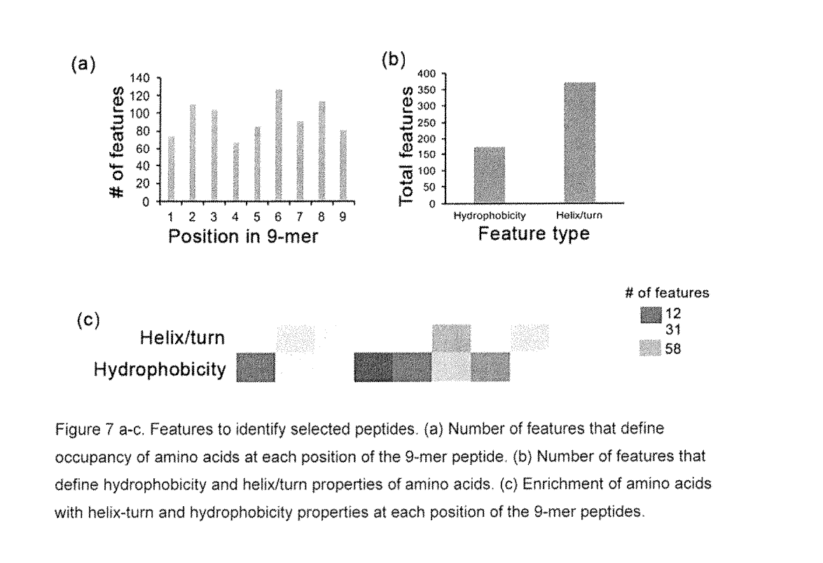

Frequently occurring features at each position of the 9-mer peptide was computed from Ensemble3 classifier and shown in FIG. 7. Names of features defining hydrophobic and helix/turn properties of amino acids are shown in Table 11.

TABLE 1
Cancer vaccines from recurrently occurring
mutations across human cancers

| | | | | |
|---|---|---|---|---|
| LQVDQLWDV | SDAYPSAFP | YPVQRLPFS | GSVSFGTVY | TGQATPLPV |
| RTFCLLVVV | RQGRQRRVR | RWLLVSSPP | VQGRVPTLE | AFWRSLLAC |
| QLREASPWV | LLRQGRQRR | FWRSLLACC | PQARAVHLP | YSTMVFLPW |
| CLLVVVVVV | VGQRIGSVS | VVVVFAVCW | LSRPGLLRQ | VDQLWDVLL |
| FCLLVVVVV | VGRSVAIGP | TCNSRQAAL | LREASPWVR | RPQLRRWLL |
| PIYMYSTMV | ELHSLWTCD | PVQRLPFST | RPEVRKTAS | LQLREASPW |
| LVVVVVVFA | SPWVRPRRR | ALSRPGLLR | LHGRADLIR | HSLWTCDCE |
| TAFWRSLLA | PLPGRIEVR | EPIYMYSTM | QGRVPTLER | LPGRIEVRT |
| QLWDVLLSR | TPEVQGRVP | VVGRSVAIG | HDPQARAVH | LWDVLLSRE |
| VQRLPFSTV | PWVRPRRRL | HGRADLIRL | PGLLRQGRQ | EVQGRVPTL |
| PQLRRWLLV | VVVVVVFAV | SGVGKSALT | IGSVSFGTV | ATVTAFWRS |
| LLVVVVVVF | WLLVSSPPS | RYPVQRLPF | VVVVVFAVC | QVDQLWDVL |
| TFCLLVVVV | LVVGRSVAI | DLIRLLLKH | VHLPELLSL | ASDAYPSAF |
| GQATPLPVT | RIGSVSFGT | ADLIRLLLK | QLRRWLLVS | DGLVVGRSV |
| TMRPLPGRI | RADLIRLLL | LHSLWTCDC | GQRIGSVSF | SGELHSLWT |
| VLLSRELFR | TVGQRIGSV | VAIGPREQW | GELHSLWTC | DQLWDVLLS |
| QATPLPVTI | RTPEVQGRV | LIRLLLHKG | RTMRPLPGR | FQDHKPKIS |
| IYMYSTMVF | RSLLACCQL | SATVTAFWR | MYSTMVFLP | |

TABLE 2
HLA Class I: List of HLA class I alleles

| | #of | | #of | | |
|---|---|---|---|---|---|
| HLA A | subtypes | HLA B | subtypes | HLA C | #of subtypes |
| HLA-A01 | 52 | HLA-B07 | 111 | HLA-C01 | 38 |
| HLA-A02 | 247 | HLA-B08 | 58 | HLA-C02 | 37 |
| HLA-A03 | 76 | HLA-B13 | 35 | HLA-C03 | 92 |
| HLA-A11 | 60 | HLA-B14 | 17 | HLA-C04 | 65 |
| HLA-A23 | 22 | HLA-B15 | 189 | HLA-C05 | 43 |
| HLA-A24 | 128 | HLA-B18 | 47 | HLA-C06 | 43 |
| HLA-A25 | 12 | HLA-B27 | 64 | HLA-C07 | 141 |
| HLA-A26 | 47 | HLA-B35 | 137 | HLA-C08 | 34 |
| HLA-A29 | 21 | HLA-B37 | 21 | HLA-C12 | 41 |
| HLA-A30 | 37 | HLA-B38 | 23 | HLA-C14 | 18 |
| HLA-A31 | 36 | HLA-B39 | 56 | HLA-C15 | 32 |
| HLA-A32 | 23 | HLA-B40 | 128 | HLA-C16 | 23 |
| HLA-A33 | 30 | | | HLA-C17 | 7 |
| HLA-A34 | 8 | | | HLA-C18 | 3 |
| HLA-A36 | 5 | | | | |
| HLA-A43 | 1 | | | | |
| HLA-A66 | 15 | | | | |
| HLA-A68 | 51 | | | | |
| HLA-A69 | 1 | | | | |
| HLA-A74 | 12 | | | | |
| HLA-A80 | 2 | | | | |

TABLE 3
HLA Class II: List of HLA class II alleles available
in netMHCcons tool for analysis

| HLA DR | HLA DQ | HLA DP |
|---|---|---|
| HLA-DRB1*01:01 | HLA-DQA1*05:01/DQB1*02:01 | HLA-DPA1*02:01/DPB1*01:01 |
| HLA-DRB1*03:01 | HLA-DQA1*05:01/DQB1*03:01 | HLA-DPA1*01:03/DPB1*02:01 |
| HLA-DRB1*04:01 | HLA-DQA1*03:01/DQB1*03:02 | HLA-DPA1*01/DPB1*04:01 |
| HLA-DRB1*04:05 | HLA-DQA1*04:01/DQB1*04:02 | HLA-DPA1*03:01/DPB1*04:02 |
| HLA-DRB1*07:01 | HLA-DQA1*01:01/DQB1*05:01 | HLA-DPA1*02:01/DPB1*05:01 |
| HLA-DRB1*08:02 | HLA-DQA1*01:02/DQB1*06:02 | HLA-DPA1*02:01/DPB1*14:01 |
| HLA-DRB1*09:01 | | |
| HLA-DRB1*11:01 | | |
| HLA-DRB1*12:01 | | |
| HLA-DRB1*13:02 | | |
| HLA-DRB1*15:01 | | |
| HLA-DRB3*01:01 | | |
| HLA-DRB3*02:02 | | |
| HLA-DRB4*01:01 | | |
| HLA-DRB5*01:01 | | |

**In the case of class I molecules, beta-chain (i.e. beta-2 microglobulin) is fixed while alpha-chain is variable. Hence, class I molecules are named based on their alpha-chains. In contrast, both alpha and beta-chains of class II molecules can vary. Thus, names of the two chains are needed to specify a class II molecules (e.g. HLA-DPA1*01:03/HLA-DPB1*02:01). For DR locus however, alpha chains are not variable. Hence, names for DR molecules use only those of the beta-chain (e.g. HLA-DRB1*01:01).

TABLE 4
List of HLA-A subtypes against which binding affinity of
peptides can be calculated

HLA-
A01:01
HLA-
A01:02
HLA-
A01:03
HLA-
A01:06
HLA-
A01:07
HLA-
A01:08
HLA-
A01:09
HLA-
A01:10
HLA-
A01:12
HLA-
A01:13
HLA-
A01:14
HLA-
A01:17
HLA-
A01:19
HLA-
A01:20
HLA-
A01:21
HLA-
A01:23
HLA-
A01:24
HLA-
A01:25
HLA-
A01:26
HLA-
A01:28
HLA-
A01:29
HLA-
A01:30
HLA-
A01:32
HLA-
A01:33
HLA-
A01:35
HLA-
A01:36
HLA-
A01:37
HLA-
A01:38
HLA-
A01:39
HLA-
A01:40
HLA-
A01:41
HLA-
A01:42
HLA-
A01:43
HLA-
A01:44
HLA-
A01:45
HLA-
A01:46
HLA-
A01:47
HLA-
A01:48
HLA-
A01:49
HLA-
A01:50
HLA-
A01:51
HLA-
A01:54
HLA-
A01:55
HLA-
A01:58
HLA-
A01:59
HLA-
A01:60
HLA-
A01:61
HLA-
A01:62
HLA-
A01:63
HLA-
A01:64
HLA-
A01:65
HLA-
A01:66
HLA-
A02:01
HLA-
A02:02
HLA-
A02:03
HLA-
A02:04
HLA-
A02:05
HLA-
A02:06
HLA-
A02:07
HLA-
A02:08
HLA-
A02:09
HLA-
A02:10
HLA-
A02:11
HLA-
A02:12
HLA-
A02:13
HLA-
A02:14
HLA-
A02:16
HLA-
A02:17
HLA-
A02:18
HLA-
A02:19
HLA-
A02:20
HLA-
A02:21
HLA-
A02:22
HLA-
A02:24
HLA-
A02:25
HLA-
A02:26
HLA-
A02:27
HLA-
A02:28
HLA-
A02:29
HLA-
A02:30
HLA-
A02:31
HLA-
A02:33
HLA-
A02:34
HLA-
A02:35
HLA-
A02:36
HLA-
A02:37
HLA-
A02:38
HLA-
A02:39
HLA-
A02:40
HLA-
A02:41
HLA-
A02:42
HLA-
A02:44
HLA-
A02:45
HLA-
A02:46
HLA-
A02:47
HLA-
A02:48
HLA-
A02:49
HLA-
A02:50
HLA-
A02:51
HLA-
A02:52
HLA-
A02:54
HLA-
A02:55
HLA-
A02:56
HLA-
A02:57
HLA-
A02:58
HLA-
A02:59
HLA-
A02:60
HLA-
A02:61
HLA-
A02:62
HLA-
A02:63
HLA-
A02:64
HLA-
A02:65
HLA-
A02:66
HLA-
A02:67
HLA-
A02:68
HLA-
A02:69
HLA-
A02:70
HLA-
A02:71
HLA-
A02:72
HLA-
A02:73
HLA-
A02:74
HLA-
A02:75
HLA-
A02:76
HLA-
A02:77
HLA-
A02:78
HLA-
A02:79
HLA-
A02:80
HLA-
A02:81
HLA-
A02:84
HLA-
A02:85
HLA-
A02:86
HLA-
A02:87
HLA-
A02:89
HLA-
A02:90
HLA-
A02:91
HLA-
A02:92
HLA-
A02:93
HLA-
A02:95
HLA-
A02:96
HLA-
A02:97
HLA-
A02:99
HLA-
A02:101
HLA-
A02:102
HLA-
A02:103
HLA-
A02:104
HLA-
A02:105
HLA-
A02:106
HLA-
A02:107
HLA-
A02:108
HLA-
A02:109
HLA-
A02:110
HLA-
A02:111
HLA-
A02:112
HLA-
A02:114
HLA-
A02:115
HLA-
A02:116
HLA-
A02:117
HLA-
A02:118
HLA-
A02:119
HLA-
A02:120
HLA-
A02:121
HLA-
A02:122
HLA-
A02:123
HLA-
A02:124
HLA-
A02:126
HLA-
A02:127
HLA-
A02:128
HLA-
A02:129
HLA-
A02:130
HLA-
A02:131
HLA-
A02:132
HLA-
A02:133
HLA-
A02:134
HLA-
A02:135
HLA-
A02:136
HLA-
A02:137
HLA-
A02:138
HLA-
A02:139
HLA-
A02:140
HLA-
A02:141
HLA-
A02:142
HLA-
A02:143
HLA-
A02:144
HLA-
A02:145
HLA-
A02:146
HLA-
A02:147
HLA-
A02:148
HLA-
A02:149
HLA-
A02:150
HLA-
A02:151
HLA-
A02:152
HLA-
A02:153
HLA-
A02:154
HLA-
A02:155
HLA-
A02:156
HLA-
A02:157
HLA-
A02:158
HLA-
A02:159
HLA-
A02:160
HLA-
A02:161
HLA-
A02:162
HLA-
A02:163
HLA-
A02:164
HLA-
A02:165
HLA-
A02:166
HLA-
A02:167
HLA-
A02:168
HLA-
A02:169
HLA-
A02:170
HLA-
A02:171
HLA-
A02:172
HLA-
A02:173
HLA-
A02:174
HLA-
A02:175
HLA-
A02:176
HLA-
A02:177
HLA-
A02:178
HLA-
A02:179
HLA-
A02:180
HLA-
A02:181
HLA-
A02:182
HLA-
A02:183
HLA-
A02:184
HLA-
A02:185
HLA-
A02:186
HLA-
A02:187
HLA-
A02:188
HLA-
A02:189
HLA-
A02:190
HLA-
A02:191
HLA-
A02:192
HLA-
A02:193
HLA-
A02:194
HLA-
A02:195
HLA-
A02:196
HLA-
A02:197
HLA-
A02:198
HLA-
A02:199
HLA-
A02:200
HLA-
A02:201
HLA-
A02:202
HLA-
A02:203
HLA-
A02:204
HLA-
A02:205
HLA-
A02:206
HLA-
A02:207
HLA-
A02:208
HLA-
A02:209
HLA-
A02:210
HLA-
A02:211
HLA-
A02:212
HLA-
A02:213
HLA-
A02:214
HLA-
A02:215
HLA-
A02:216
HLA-
A02:217
HLA-
A02:218
HLA-
A02:219
HLA-
A02:220
HLA-
A02:221
HLA-
A02:224
HLA-
A02:228
HLA-
A02:229
HLA-
A02:230
HLA-
A02:231
HLA-
A02:232
HLA-
A02:233
HLA-
A02:234
HLA-
A02:235
HLA-
A02:236
HLA-
A02:237
HLA-
A02:238
HLA-
A02:239
HLA-
A02:240
HLA-
A02:241
HLA-
A02:242
HLA-
A02:243
HLA-
A02:244
HLA-
A02:245
HLA-
A02:246
HLA-
A02:247
HLA-
A02:248
HLA-
A02:249
HLA-
A02:251
HLA-
A02:252
HLA-
A02:253
HLA-
A02:254
HLA-
A02:255
HLA-
A02:256
HLA-
A02:257
HLA-
A02:258
HLA-
A02:259
HLA-
A02:260
HLA-
A02:261
HLA-
A02:262
HLA-
A02:263
HLA-
A02:264
HLA-
A02:265
HLA-
A02:266
HLA-
A03:01
HLA-
A03:02
HLA-
A03:04
HLA-
A03:05
HLA-
A03:06
HLA-
A03:07
HLA-
A03:08
HLA-
A03:09
HLA-
A03:10
HLA-
A03:12
HLA-
A03:13
HLA-
A03:14
HLA-
A03:15
HLA-
A03:16
HLA-
A03:17
HLA-
A03:18
HLA-
A03:19
HLA-
A03:20
HLA-
A03:22
HLA-
A03:23
HLA-
A03:24
HLA-
A03:25
HLA-
A03:26
HLA-
A03:27
HLA-
A03:28
HLA-
A03:29
HLA-
A03:30
HLA-
A03:31
HLA-
A03:32
HLA-
A03:33
HLA-
A03:34
HLA-
A03:35
HLA-
A03:37
HLA-
A03:38
HLA-
A03:39
HLA-
A03:40
HLA-
A03:41
HLA-
A03:42
HLA-
A03:43
HLA-
A03:44
HLA-
A03:45
HLA-
A03:46
HLA-
A03:47
HLA-
A03:48
HLA-
A03:49
HLA-
A03:50
HLA-
A03:51
HLA-
A03:52
HLA-
A03:53
HLA-
A03:54
HLA-
A03:55
HLA-
A03:56
HLA-
A03:57
HLA-
A03:58
HLA-
A03:59
HLA-
A03:60
HLA-
A03:61
HLA-
A03:62
HLA-
A03:63
HLA-
A03:64
HLA-
A03:65
HLA-
A03:66
HLA-
A03:67
HLA-
A03:70
HLA-
A03:71
HLA-
A03:72
HLA-
A03:73
HLA-
A03:74
HLA-
A03:75
HLA-
A03:76
HLA-
A03:77
HLA-
A03:78
HLA-
A03:79
HLA-
A03:80
HLA-
A03:81
HLA-
A03:82
HLA-
A11:01
HLA-
A11:02
HLA-
A11:03
HLA-
A11:04
HLA-
A11:05
HLA-
A11:06
HLA-
A11:07
HLA-
A11:08
HLA-
A11:09
HLA-
Al 1:10
HLA-
A11:11
HLA-
A11:12
HLA-
A11:13
HLA-
A11:14
HLA-
A11:15
HLA-
A11:16
HLA-
A11:17
HLA-
A11:18
HLA-
A11:19
HLA-
A11:20
HLA-
A11:22
HLA-
A11:23
HLA-
A11:24
HLA-
A11:25
HLA-
A11:26
HLA-
A11:27
HLA-
A11:29
HLA-
A11:30
HLA-
A11:31
HLA-
A11:32
HLA-
A11:33
HLA-
A11:34
HLA-
A11:35
HLA-
A11:36
HLA-
A11:37
HLA-
A11:38
HLA-
A11:39
HLA-
A11:40
HLA-
A11:41
HLA-
A11:42
HLA-
Al 1:43
HLA-
A11:44
HLA-
A11:45
HLA-
A11:46
HLA-
A11:47
HLA-
A11:48
HLA-
A11:49
HLA-
A11:51
HLA-
A11:53
HLA-
A11:54
HLA-
A11:55
HLA-
A11:56
HLA-
A11:57
HLA-
A11:58
HLA-
A11:59
HLA-
A11:60
HLA-
A11:61
HLA-
A11:62
HLA-
A11:63
HLA-
A11:64
HLA-
A23:01
HLA-
A23:02
HLA-
A23:03
HLA-
A23:04
HLA-
A23:05
HLA-
A23:06
HLA-
A23:09
HLA-
A23:10
HLA-
A23:12
HLA-
A23:13
HLA-
A23:14
HLA-
A23:15
HLA-
A23:16
HLA-
A23:17
HLA-
A23:18
HLA-
A23:20
HLA-
A23:21
HLA-
A23:22
HLA-
A23:23
HLA-
A23:24
HLA-
A23:25
HLA-
A23:26
HLA-
A24:02
HLA-
A24:03
HLA-
A24:04
HLA-
A24:05
HLA-
A24:06
HLA-
A24:07
HLA-
A24:08
HLA-
A24:10
HLA-
A24:13
HLA-
A24:14
HLA-
A24:15
HLA-
A24:17
HLA-
A24:18
HLA-
A24:19
HLA-
A24:20
HLA-
A24:21
HLA-
A24:22
HLA-
A24:23
HLA-
A24:24
HLA-
A24:25
HLA-
A24:26
HLA-
A24:27
HLA-
A24:28
HLA-
A24:29
HLA-
A24:30
HLA-
A24:31
HLA-
A24:32
HLA-
A24:33
HLA-
A24:34
HLA-
A24:35
HLA-
A24:37
HLA-
A24:38
HLA-
A24:39
HLA-
A24:41
HLA-
A24:42
HLA-
A24:43
HLA-
A24:44
HLA-
A24:46
HLA-
A24:47
HLA-
A24:49
HLA-
A24:50
HLA-
A24:51
HLA-
A24:52
HLA-
A24:53
HLA-
A24:54
HLA-
A24:55
HLA-
A24:56
HLA-
A24:57
HLA-
A24:58
HLA-
A24:59
HLA-
A24:61
HLA-
A24:62
HLA-
A24:63
HLA-
A24:64
HLA-
A24:66
HLA-
A24:67
HLA-
A24:68
HLA-
A24:69
HLA-
A24:70
HLA-
A24:71
HLA-
A24:72
HLA-
A24:73
HLA-
A24:74
HLA-
A24:75
HLA-
A24:76
HLA-
A24:77
HLA-
A24:78
HLA-
A24:79
HLA-
A24:80
HLA-
A24:81
HLA-
A24:82
HLA-
A24:85
HLA-
A24:87
HLA-
A24:88
HLA-
A24:89
HLA-
A24:91
HLA-
A24:92
HLA-
A24:93
HLA-
A24:94
HLA-
A24:95
HLA-
A24:96
HLA-
A24:97
HLA-
A24:98
HLA-
A24:99
HLA-
A24:100
HLA-
A24:101
HLA-
A24:102
HLA-
A24:103
HLA-
A24:104
HLA-
A24:105
HLA-
A24:106
HLA-
A24:107
HLA-
A24:108
HLA-
A24:109
HLA-
A24:110
HLA-
A24:111
HLA-
A24:112
HLA-
A24:113
HLA-
A24:114
HLA-
A24:115
HLA-
A24:116
HLA-
A24:117
HLA-
A24:118
HLA-
A24:119
HLA-
A24:120
HLA-
A24:121
HLA-
A24:122
HLA-
A24:123
HLA-
A24:124
HLA-
A24:125
HLA-
A24:126
HLA-
A24:127
HLA-
A24:128
HLA-
A24:129
HLA-
A24:130
HLA-
A24:131
HLA-
A24:133
HLA-
A24:134
HLA-
A24:135
HLA-
A24:136
HLA-
A24:137
HLA-
A24:138
HLA-
A24:139
HLA-
A24:140
HLA-
A24:141
HLA-
A24:142
HLA-
A24:143
HLA-
A24:144
HLA-
A25:01
HLA-
A25:02
HLA-
A25:03
HLA-
A25:04
HLA-
A25:05
HLA-
A25:06
HLA-
A25:07
HLA-
A25:08
HLA-
A25:09
HLA-
A25:10
HLA-
A25:11
HLA-
A25:13
HLA-
A26:01
HLA-
A26:02
HLA-
A26:03
HLA-
A26:04
HLA-
A26:05
HLA-
A26:06
HLA-
A26:07
HLA-
A26:08
HLA-
A26:09
HLA-
A26:10
HLA-
A26:12
HLA-
A26:13
HLA-
A26:14
HLA-
A26:15
HLA-
A26:16
HLA-
A26:17
HLA-
A26:18
HLA-
A26:19
HLA-
A26:20
HLA-
A26:21
HLA-
A26:22
HLA-
A26:23
HLA-
A26:24
HLA-
A26:26
HLA-
A26:27
HLA-
A26:28
HLA-
A26:29
HLA-
A26:30
HLA-
A26:31
HLA-
A26:32
HLA-
A26:33
HLA-
A26:34
HLA-
A26:35
HLA-
A26:36
HLA-
A26:37
HLA-
A26:38
HLA-
A26:39
HLA-
A26:40
HLA-
A26:41
HLA-
A26:42
HLA-
A26:43
HLA-
A26:45
HLA-
A26:46
HLA-
A26:47
HLA-
A26:48
HLA-
A26:49
HLA-
A26:50
HLA-
A29:01
HLA-
A29:02
HLA-
A29:03
HLA-
A29:04
HLA-
A29:05
HLA-
A29:06
HLA-
A29:07
HLA-
A29:09
HLA-
A29:10
HLA-
A29:11
HLA-
A29:12
HLA-
A29:13
HLA-
A29:14
HLA-
A29:15
HLA-
A29:16
HLA-
A29:17
HLA-
A29:18
HLA-
A29:19
HLA-
A29:20
HLA-
A29:21
HLA-
A29:22
HLA-
A30:01
HLA-
A30:02
HLA-
A30:03
HLA-
A30:04
HLA-
A30:06
HLA-
A30:07
HLA-
A30:08
HLA-
A30:09
HLA-
A30:10
HLA-
A30:11
HLA-
A30:12
HLA-
A30:13
HLA-
A30:15
HLA-
A30:16
HLA-
A30:17
HLA-
A30:18
HLA-
A30:19
HLA-
A30:20
HLA-
A30:22
HLA-
A30:23
HLA-
A30:24
HLA-
A30:25
HLA-
A30:26
HLA-
A30:28
HLA-
A30:29
HLA-
A30:30
HLA-
A30:31
HLA-
A30:32
HLA-
A30:33
HLA-
A30:34
HLA-
A30:35
HLA-
A30:36
HLA-
A30:37
HLA-
A30:38
HLA-
A30:39
HLA-
A30:40
HLA-
A30:41
HLA-
A31:01
HLA-
A31:02
HLA-
A31:03
HLA-
A31:04
HLA-
A31:05
HLA-
A31:06
HLA-
A31:07
HLA-
A31:08
HLA-
A31:09
HLA-
A31:10
HLA-
A31:11
HLA-
A31:12
HLA-
A31:13
HLA-
A31:15
HLA-
A31:16
HLA-
A31:17
HLA-
A31:18
HLA-
A31:19
HLA-
A31:20
HLA-
A31:21
HLA-
A31:22
HLA-
A31:23
HLA-
A31:24
HLA-
A31:25
HLA-
A31:26
HLA-
A31:27
HLA-
A31:28
HLA-
A31:29
HLA-
A31:30
HLA-
A31:31
HLA-
A31:32
HLA-
A31:33
HLA-
A31:34
HLA-
A31:35
HLA-
A31:36
HLA-
A31:37
HLA-
A32:01
HLA-
A32:02
HLA-
A32:03
HLA-
A32:04
HLA-
A32:05
HLA-
A32:06
HLA-
A32:07
HLA-
A32:08
HLA-
A32:09
HLA-
A32:10
HLA-
A32:12
HLA-
A32:13
HLA-
A32:14
HLA-
A32:15
HLA-
A32:16
HLA-
A32:17
HLA-
A32:18
HLA-
A32:20
HLA-
A32:21
HLA-
A32:22
HLA-
A32:23
HLA-
A32:24
HLA-
A32:25
HLA-
A33:01
HLA-
A33:03
HLA-
A33:04
HLA-
A33:05
HLA-
A33:06
HLA-
A33:07
HLA-
A33:08
HLA-
A33:09
HLA-
A33:10
HLA-
A33:11
HLA-
A33:12
HLA-
A33:13
HLA-
A33:14
HLA-
A33:15
HLA-
A33:16
HLA-
A33:17
HLA-
A33:18
HLA-
A33:19
HLA-
A33:20
HLA-
A33:21
HLA-
A33:22
HLA-
A33:23
HLA-
A33:24
HLA-
A33:25
HLA-
A33:26
HLA-
A33:27
HLA-
A33:28
HLA-
A33:29
HLA-
A33:30
HLA-
A33:31
HLA-
A34:01
HLA-
A34:02
HLA-
A34:03
HLA-
A34:04
HLA-
A34:05
HLA-
A34:06
HLA-
A34:07
HLA-
A34:08
HLA-
A36:01
HLA-
A36:02
HLA-
A36:03
HLA-
A36:04
HLA-
A36:05
HLA-
A43:01
HLA-
A66:01
HLA-
A66:02
HLA-
A66:03
HLA-
A66:04
HLA-
A66:05
HLA-
A66:06
HLA-
A66:07
HLA-
A66:08
HLA-
A66:09
HLA-
A66:10
HLA-
A66:11
HLA-
A66:12
HLA-
A66:13
HLA-
A66:14
HLA-
A66:15
HLA-
A68:01
HLA-
A68:02
HLA-
A68:03
HLA-
A68:04
HLA-
A68:05
HLA-
A68:06
HLA-
A68:07
HLA-
A68:08
HLA-
A68:09
HLA-
A68:10
HLA-
A68:12
HLA-
A68:13
HLA-
A68:14
HLA-
A68:15
HLA-
A68:16
HLA-
A68:17
HLA-
A68:19
HLA-
A68:20
HLA-
A68:21
HLA-
A68:22
HLA-
A68:23
HLA-
A68:24
HLA-
A68:25
HLA-
A68:26
HLA-
A68:27
HLA-
A68:28
HLA-
A68:29
HLA-
A68:30
HLA-
A68:31
HLA-
A68:32
HLA-
A68:33
HLA-
A68:34
HLA-
A68:35
HLA-
A68:36
HLA-
A68:37
HLA-
A68:38
HLA-
A68:39
HLA-
A68:40
HLA-
A68:41
HLA-
A68:42
HLA-
A68:43
HLA-
A68:44
HLA-
A68:45
HLA-
A68:46
HLA-
A68:47
HLA-
A68:48
HLA-
A68:50
HLA-
A68:51
HLA-
A68:52
HLA-
A68:53
HLA-
A68:54
HLA-
A69:01
HLA-
A74:01
HLA-
A74:02
HLA-
A74:03
HLA-
A74:04
HLA-
A74:05
HLA-
A74:06
HLA-
A74:07
HLA-
A74:08
HLA-
A74:09
HLA-
A74:10
HLA-
A74:11
HLA-
A74:13
HLA-
A80:01
HLA-
A80:02

TABLE 5
List of HLA-B subtypes against which binding affinity of
peptides are calculated

HLA-
B07:02
HLA-
B07:03
HLA-
B07:04
HLA-
B07:05
HLA-
B07:06
HLA-
B07:07
HLA-
B07:08
HLA-
B07:09
HLA-
B07:10
HLA-
B07:11
HLA-
B07:12
HLA-
B07:13
HLA-
B07:14
HLA-
B07:15
HLA-
B07:16
HLA-
B07:17
HLA-
B07:18
HLA-
B07:19
HLA-
B07:20
HLA-
B07:21
HLA-
B07:22
HLA-
B07:23
HLA-
B07:24
HLA-
B07:25
HLA-
B07:26
HLA-
B07:27
HLA-
B07:28
HLA-
B07:29
HLA-
B07:30
HLA-
B07:31
HLA-
B07:32
HLA-
B07:33
HLA-
B07:34
HLA-
B07:35
HLA-
B07:36
HLA-
B07:37
HLA-
B07:38
HLA-
B07:39
HLA-
B07:40
HLA-
B07:41
HLA-
B07:42
HLA-
B07:43
HLA-
B07:44
HLA-
B07:45
HLA-
B07:46
HLA-
B07:47
HLA-
B07:48
HLA-
B07:50
HLA-
B07:51
HLA-
B07:52
HLA-
B07:53
HLA-
B07:54
HLA-
B07:55
HLA-
B07:56
HLA-
B07:57
HLA-
B07:58
HLA-
B07:59
HLA-
B07:60
HLA-
B07:61
HLA-
B07:62
HLA-
B07:63
HLA-
B07:64
HLA-
B07:65
HLA-
B07:66
HLA-
B07:68
HLA-
B07:69
HLA-
B07:70
HLA-
B07:71
HLA-
B07:72
HLA-
B07:73
HLA-
B07:74
HLA-
B07:75
HLA-
B07:76
HLA-
B07:77
HLA-
B07:78
HLA-
B07:79
HLA-
B07:80
HLA-
B07:81
HLA-
B07:82
HLA-
B07:83
HLA-
B07:84
HLA-
B07:85
HLA-
B07:86
HLA-
B07:87
HLA-
B07:88
HLA-
B07:89
HLA-
B07:90
HLA-
B07:91
HLA-
B07:92
HLA-
B07:93
HLA-
B07:94
HLA-
B07:95
HLA-
B07:96
HLA-
B07:97
HLA-
B07:98
HLA-
B07:99
HLA-
B07:100
HLA-
B07:101
HLA-
B07:102
HLA-
B07:103
HLA-
B07:104
HLA-
B07:105
HLA-
B07:106
HLA-
B07:107
HLA-
B07:108
HLA-
B07:109
HLA-
B07:110
HLA-
B07:112
HLA-
B07:113
HLA-
B07:114
HLA-
B07:115
HLA-B08:01
HLA-B08:02
HLA-B08:03
HLA-B08:04
HLA-B08:05
HLA-B08:07
HLA-B08:09
HLA-B08:10
HLA-B08:11
HLA-B08:12
HLA-B08:13
HLA-B08:14
HLA-B08:15
HLA-B08:16
HLA-B08:17
HLA-B08:18
HLA-B08:20
HLA-B08:21
HLA-B08:22
HLA-B08:23
HLA-B08:24
HLA-B08:25
HLA-B08:26
HLA-B08:27
HLA-B08:28
HLA-B08:29
HLA-B08:31
HLA-B08:32
HLA-B08:33
HLA-B08:34
HLA-B08:35
HLA-B08:36
HLA-B08:37
HLA-B08:38
HLA-B08:39
HLA-B08:40
HLA-B08:41
HLA-B08:42
HLA-B08:43
HLA-B08:44
HLA-B08:45
HLA-B08:46
HLA-B08:47
HLA-B08:48
HLA-B08:49
HLA-B08:50
HLA-B08:51
HLA-B08:52
HLA-B08:53
HLA-B08:54
HLA-B08:55
HLA-B08:56
HLA-B08:57
HLA-B08:58
HLA-B08:59
HLA-B08:60
HLA-B08:61
HLA-B08:62
HLA-B13:01
HLA-B13:02
HLA-B13:03
HLA-B13:04
HLA-B13:06
HLA-B13:09
HLA-B13:10
HLA-B13:11
HLA-B13:12
HLA-B13:13
HLA-B13:14
HLA-B13:15
HLA-B13:16
HLA-B13:17
HLA-B13:18
HLA-B13:19
HLA-B13:20
HLA-B13:21
HLA-B13:22
HLA-B13:23
HLA-B13:25
HLA-B13:26
HLA-B13:27
HLA-B13:28
HLA-B13:29
HLA-B13:30
HLA-B13:31
HLA-B13:32
HLA-B13:33
HLA-B13:34
HLA-
B13:35
HLA-
B13:36
HLA-
B13:37
HLA-
B13:38
HLA-
B13:39
HLA-
B14:01
HLA-
B14:02
HLA-
B14:03
HLA-
B14:04
HLA-
B14:05
HLA-
B14:06
HLA-
B14:08
HLA-
B14:09
HLA-
B14:10
HLA-
B14:11
HLA-
B14:12
HLA-
B14:13
HLA-
B14:14
HLA-
B14:15
HLA-
B14:16
HLA-
B14:17
HLA-
B14:18
HLA-
B15:01
HLA-
B15:02
HLA-
B15:03
HLA-
B15:04
HLA-
B15:05
HLA-
B15:06
HLA-
B15:07
HLA-
B15:08
HLA-
B15:09
HLA-
B15:10
HLA-
B15:11
HLA-
B15:12
HLA-
B15:13
HLA-
B15:14
HLA-
B15:15
HLA-
B15:16
HLA-
B15:17
HLA-
B15:18
HLA-
B15:19
HLA-
B15:20
HLA-
B15:21
HLA-
B15:23
HLA-
B15:24
HLA-
B15:25
HLA-
B15:27
HLA-
B15:28
HLA-
B15:29
HLA-
B15:30
HLA-
B15:31
HLA-
B15:32
HLA-
B15:33
HLA-
B15:34
HLA-
B15:35
HLA-
B15:36
HLA-
B15:37
HLA-
B15:38
HLA-
B15:39
HLA-
B15:40
HLA-
B15:42
HLA-
B15:43
HLA-
B15:44
HLA-
B15:45
HLA-
B15:46
HLA-
B15:47
HLA-
B15:48
HLA-
B15:49
HLA-
B15:50
HLA-
B15:51
HLA-
B15:52
HLA-
B15:53
HLA-
B15:54
HLA-
B15:55
HLA-
B15:56
HLA-
B15:57
HLA-
B15:58
HLA-
B15:60
HLA-
B15:61
HLA-
B15:62
HLA-
B15:63
HLA-
B15:64
HLA-
B15:65
HLA-
B15:66
HLA-
B15:67
HLA-
B15:68
HLA-
B15:69
HLA-
B15:70
HLA-
B15:71
HLA-
B15:72
HLA-
B15:73
HLA-
B15:74
HLA-
B15:75
HLA-
B15:76
HLA-
B15:77
HLA-
B15:78
HLA-
B15:80
HLA-
B15:81
HLA-
B15:82
HLA-
B15:83
HLA-
B15:84
HLA-
B15:85
HLA-
B15:86
HLA-
B15:87
HLA-
B15:88
HLA-
B15:89
HLA-
B15:90
HLA-
B15:91
HLA-
B15:92
HLA-
B15:93
HLA-
B15:95
HLA-
B15:96
HLA-
B15:97
HLA-
B15:98
HLA-
B15:99
HLA-
B15:101
HLA-
B15:102
HLA-
B15:103
HLA-
B15:104
HLA-
B15:105
HLA-
B15:106
HLA-
B15:107
HLA-
B15:108
HLA-
B15:109
HLA-
B15:110
HLA-
B15:112
HLA-
B15:113
HLA-
B15:114
HLA-
B15:115
HLA-
B15:116
HLA-
B15:117
HLA-
B15:118
HLA-
B15:119
HLA-
B15:120
HLA-
B15:121
HLA-
B15:122
HLA-
B15:123
HLA-
B15:124
HLA-
B15:125
HLA-
B15:126
HLA-
B15:127
HLA-
B15:128
HLA-
B15:129
HLA-
B15:131
HLA-
B15:132
HLA-
B15:133
HLA-
B15:134
HLA-
B15:135
HLA-
B15:136
HLA-
B15:137
HLA-
B15:138
HLA-
B15:139
HLA-
B15:140
HLA-
B15:141
HLA-
B15:142
HLA-
B15:143
HLA-
B15:144
HLA-
B15:145
HLA-
B15:146
HLA-
B15:147
HLA-
B15:148
HLA-
B15:150
HLA-
B15:151
HLA-
B15:152
HLA-
B15:153
HLA-
B15:154
HLA-
B15:155
HLA-
B15:156
HLA-
B15:157
HLA-
B15:158
HLA-
B15:159
HLA-
B15:160
HLA-
B15:161
HLA-
B15:162
HLA-
B15:163
HLA-
B15:164
HLA-
B15:165
HLA-
B15:166
HLA-
B15:167
HLA-
B15:168
HLA-
B15:169
HLA-
B15:170
HLA-
B15:171
HLA-
B15:172
HLA-
B15:173
HLA-
B15:174
HLA-
B15:175
HLA-
B15:176
HLA-
B15:177
HLA-
B15:178
HLA-
B15:179
HLA-
B15:180
HLA-
B15:183
HLA-
B15:184
HLA-
B15:185
HLA-
B15:186
HLA-
B15:187
HLA-
B15:188
HLA-
B15:189
HLA-
B15:191
HLA-
B15:192
HLA-
B15:193
HLA-
B15:194
HLA-
B15:195
HLA-
B15:196
HLA-
B15:197
HLA-
B15:198
HLA-
B15:199
HLA-
B15:200
HLA-
B15:201
HLA-
B15:202
HLA-B18:01
HLA-B18:02
HLA-B18:03
HLA-B18:04
HLA-B18:05
HLA-B18:06
HLA-B18:07
HLA-B18:08
HLA-B18:09
HLA-B18:10
HLA-B18:11
HLA-B18:12
HLA-B18:13
HLA-B18:14
HLA-B18:15
HLA-B18:18
HLA-B18:19
HLA-B18:20
HLA-B18:21
HLA-B18:22
HLA-B18:24
HLA-B18:25
HLA-B18:26
HLA-B18:27
HLA-B18:28
HLA-B18:29
HLA-B18:30
HLA-B18:31
HLA-B18:32
HLA-B18:33
HLA-B18:34
HLA-B18:35
HLA-B18:36
HLA-B18:37
HLA-B18:38
HLA-B18:39
HLA-B18:40
HLA-B18:41
HLA-B18:42
HLA-B18:43
HLA-B18:44
HLA-B18:45
HLA-B18:46
HLA-B18:47
HLA-B18:48
HLA-B18:49
HLA-B18:50
HLA-B27:01
HLA-B27:02
HLA-B27:03
HLA-B27:04
HLA-B27:05
HLA-B27:06
HLA-B27:07
HLA-B27:08
HLA-B27:09
HLA-B27:10
HLA-B27:11
HLA-B27:12
HLA-B27:13
HLA-B27:14
HLA-B27:15
HLA-B27:16
HLA-B27:17
HLA-B27:18
HLA-B27:19
HLA-B27:20
HLA-B27:21
HLA-B27:23
HLA-B27:24
HLA-B27:25
HLA-B27:26
HLA-B27:27
HLA-B27:28
HLA-B27:29
HLA-B27:30
HLA-B27:31
HLA-B27:32
HLA-B27:33
HLA-B27:34
HLA-B27:35
HLA-B27:36
HLA-B27:37
HLA-B27:38
HLA-B27:39
HLA-B27:40
HLA-B27:41
HLA-B27:42
HLA-B27:43
HLA-
B27:44
HLA-
B27:45
HLA-
B27:46
HLA-
B27:47
HLA-
B27:48
HLA-
B27:49
HLA-
B27:50
HLA-
B27:51
HLA-
B27:52
HLA-
B27:53
HLA-
B27:54
HLA-
B27:55
HLA-
B27:56
HLA-
B27:57
HLA-
B27:58
HLA-
B27:60
HLA-
B27:61
HLA-
B27:62
HLA-
B27:63
HLA-
B27:67
HLA-
B27:68
HLA-
B27:69
HLA-
B35:01
HLA-
B35:02
HLA-
B35:03
HLA-
B35:04
HLA-
B35:05
HLA-
B35:06
HLA-
B35:07
HLA-
B35:08
HLA-
B35:09
HLA-
B35:10
HLA-
B35:11
HLA-
B35:12
HLA-
B35:13
HLA-
B35:14
HLA-
B35:15
HLA-
B35:16
HLA-
B35:17
HLA-
B35:18
HLA-
B35:19
HLA-
B35:20
HLA-
B35:21
HLA-
B35:22
HLA-
B35:23
HLA-
B35:24
HLA-
B35:25
HLA-
B35:26
HLA-
B35:27
HLA-
B35:28
HLA-
B35:29
HLA-
B35:30
HLA-
B35:31
HLA-
B35:32
HLA-
B35:33
HLA-
B35:34
HLA-
B35:35
HLA-
B35:36
HLA-
B35:37
HLA-
B35:38
HLA-
B35:39
HLA-
B35:41
HLA-
B35:42
HLA-
B35:43
HLA-
B35:44
HLA-
B35:45
HLA-
B35:46
HLA-
B35:47
HLA-
B35:48
HLA-
B35:49
HLA-
B35:50
HLA-
B35:51
HLA-
B35:52
HLA-
B35:54
HLA-
B35:55
HLA-
B35:56
HLA-
B35:57
HLA-
B35:58
HLA-
B35:59
HLA-
B35:60
HLA-
B35:61
HLA-
B35:62
HLA-
B35:63
HLA-
B35:64
HLA-
B35:66
HLA-
B35:67
HLA-
B35:68
HLA-
B35:69
HLA-
B35:70
HLA-
B35:71
HLA-
B35:72
HLA-
B35:74
HLA-
B35:75
HLA-
B35:76
HLA-
B35:77
HLA-
B35:78
HLA-
B35:79
HLA-
B35:80
HLA-
B35:81
HLA-
B35:82
HLA-
B35:83
HLA-
B35:84
HLA-
B35:85
HLA-
B35:86
HLA-
B35:87
HLA-
B35:88
HLA-
B35:89
HLA-
B35:90
HLA-
B35:91
HLA-
B35:92
HLA-
B35:93
HLA-
B35:94
HLA-
B35:95
HLA-
B35:96
HLA-
B35:97
HLA-
B35:98
HLA-
B35:99
HLA-
B35:100
HLA-
B35:101
HLA-
B35:102
HLA-
B35:103
HLA-
B35:104
HLA-
B35:105
HLA-
B35:106
HLA-
B35:107
HLA-
B35:108
HLA-
B35:109
HLA-
B35:110
HLA-
B35:111
HLA-
B35:112
HLA-
B35:113
HLA-
B35:114
HLA-
B35:115
HLA-
B35:116
HLA-
B35:117
HLA-
B35:118
HLA-
B35:119
HLA-
B35:120
HLA-
B35:121
HLA-
B35:122
HLA-
B35:123
HLA-
B35:124
HLA-
B35:125
HLA-
B35:126
HLA-
B35:127
HLA-
B35:128
HLA-
B35:131
HLA-
B35:132
HLA-
B35:133
HLA-
B35:135
HLA-
B35:136
HLA-
B35:137
HLA-
B35:138
HLA-
B35:139
HLA-
B35:140
HLA-
B35:141
HLA-
B35:142
HLA-
B35:143
HLA-
B35:144
HLA-
B37:01
HLA-
B37:02
HLA-
B37:04
HLA-
B37:05
HLA-
B37:06
HLA-
B37:07
HLA-
B37:08
HLA-
B37:09
HLA-
B37:10
HLA-
B37:11
HLA-
B37:12
HLA-
B37:13
HLA-
B37:14
HLA-
B37:15
HLA-
B37:17
HLA-
B37:18
HLA-
B37:19
HLA-
B37:20
HLA-
B37:21
HLA-
B37:22
HLA-
B37:23
HLA-
B38:01
HLA-
B38:02
HLA-
B38:03
HLA-
B38:04
HLA-
B38:05
HLA-
B38:06
HLA-
B38:07
HLA-
B38:08
HLA-
B38:09
HLA-
B38:10
HLA-
B38:11
HLA-
B38:12
HLA-
B38:13
HLA-
B38:14
HLA-
B38:15
HLA-
B38:16
HLA-
B38:17
HLA-
B38:18
HLA-
B38:19
HLA-
B38:20
HLA-
B38:21
HLA-
B38:22
HLA-
B38:23
HLA-
B39:01
HLA-
B39:02
HLA-
B39:03
HLA-
B39:04
HLA-
B39:05
HLA-
B39:06
HLA-
B39:07
HLA-
B39:08
HLA-
B39:09
HLA-
B39:10
HLA-
B39:11
HLA-
B39:12
HLA-
B39:13
HLA-
B39:14
HLA-
B39:15
HLA-
B39:16
HLA-
B39:17
HLA-
B39:18
HLA-
B39:19
HLA-
B39:20
HLA-
B39:22
HLA-
B39:23
HLA-
B39:24
HLA-
B39:26
HLA-
B39:27
HLA-
B39:28
HLA-
B39:29
HLA-
B39:30
HLA-
B39:31
HLA-
B39:32
HLA-
B39:33
HLA-
B39:34
HLA-
B39:35
HLA-
B39:36
HLA-
B39:37
HLA-
B39:39
HLA-
B39:41
HLA-
B39:42
HLA-
B39:43
HLA-
B39:44
HLA-
B39:45
HLA-
B39:46
HLA-
B39:47
HLA-
B39:48
HLA-
B39:49
HLA-
B39:50
HLA-
B39:51
HLA-
B39:52
HLA-
B39:53
HLA-
B39:54
HLA-
B39:55
HLA-
B39:56
HLA-
B39:57
HLA-
B39:58
HLA-
B39:59
HLA-
B39:60
HLA-
B40:01
HLA-
B40:02
HLA-
B40:03
HLA-
B40:04
HLA-
B40:05
HLA-
B40:06
HLA-
B40:07
HLA-
B40:08
HLA-
B40:09
HLA-
B40:10
HLA-
B40:11
HLA-
B40:12
HLA-
B40:13
HLA-
B40:14
HLA-
B40:15
HLA-
B40:16
HLA-
B40:18
HLA-
B40:19
HLA-
B40:20
HLA-
B40:21
HLA-
B40:23
HLA-
B40:24
HLA-
B40:25
HLA-
B40:26
HLA-
B40:27
HLA-
B40:28
HLA-
B40:29
HLA-
B40:30
HLA-
B40:31
HLA-
B40:32
HLA-
B40:33
HLA-
B40:34
HLA-
B40:35
HLA-
B40:36
HLA-
B40:37
HLA-
B40:38
HLA-
B40:39
HLA-
B40:40
HLA-
B40:42
HLA-
B40:43
HLA-
B40:44
HLA-
B40:45
HLA-
B40:46
HLA-
B40:47
HLA-
B40:48
HLA-
B40:49
HLA-
B40:50
HLA-
B40:51
HLA-
B40:52
HLA-
B40:53
HLA-
B40:54
HLA-
B40:55
HLA-
B40:56
HLA-
B40:57
HLA-
B40:58
HLA-
B40:59
HLA-
B40:60
HLA-
B40:61
HLA-
B40:62
HLA-
B40:63
HLA-
B40:64
HLA-
B40:65
HLA-
B40:66
HLA-
B40:67
HLA-
B40:68
HLA-
B40:69
HLA-
B40:70
HLA-
B40:71
HLA-
B40:72
HLA-
B40:73
HLA-
B40:74
HLA-
B40:75
HLA-
B40:76
HLA-
B40:77
HLA-
B40:78
HLA-
B40:79
HLA-
B40:80
HLA-
B40:81
HLA-
B40:82
HLA-
B40:83
HLA-
B40:84
HLA-
B40:85
HLA-
B40:86
HLA-
B40:87
HLA-
B40:88
HLA-
B40:89
HLA-
B40:90
HLA-
B40:91
HLA-
B40:92
HLA-
B40:93
HLA-
B40:94
HLA-
B40:95
HLA-
B40:96
HLA-
B40:97
HLA-
B40:98
HLA-
B40:99
HLA-
B40:100
HLA-
B40:101
HLA-
B40:102
HLA-
B40:103
HLA-
B40:104
HLA-
B40:105
HLA-
B40:106
HLA-
B40:107
HLA-
B40:108
HLA-
B40:109
HLA-
B40:110
HLA-
B40:111
HLA-
B40:112
HLA-
B40:113
HLA-
B40:114
HLA-
B40:115
HLA-
B40:116
HLA-
B40:117
HLA-
B40:119
HLA-
B40:120
HLA-
B40:121
HLA-
B40:122
HLA-
B40:123
HLA-
B40:124
HLA-
B40:125
HLA-
B40:126
HLA-
B40:127
HLA-
B40:128
HLA-
B40:129
HLA-
B40:130
HLA-
B40:131
HLA-
B40:132

TABLE 6
List of HLA-C subtypes against which binding affinity of
peptides are calculated

HLA-
C01:02
HLA-
C01:03
HLA-
C01:04
HLA-
C01:05
HLA-
C01:06
HLA-
C01:07
HLA-
C01:08
HLA-
C01:09
HLA-
C01:10
HLA-
C01:11
HLA-
C01:12
HLA-
C01:13
HLA-
C01:14
HLA-
C01:15
HLA-
C01:16
HLA-
C01:17
HLA-
C01:18
HLA-
C01:19
HLA-
C01:20
HLA-
C01:21
HLA-
C01:22
HLA-
C01:23
HLA-
C01:24
HLA-
C01:25
HLA-
C01:26
HLA-
C01:27
HLA-
C01:28
HLA-
C01:29
HLA-
C01:30
HLA-
C01:31
HLA-
C01:32
HLA-
C01:33
HLA-
C01:34
HLA-
C01:35
HLA-
C01:36
HLA-
C01:38
HLA-
C01:39
HLA-
C01:40
HLA-
C02:02
HLA-
C02:03
HLA-
C02:04
HLA-
C02:05
HLA-
C02:06
HLA-
C02:07
HLA-
C02:08
HLA-
C02:09
HLA-
C02:10
HLA-
C02:11
HLA-
C02:12
HLA-
C02:13
HLA-
C02:14
HLA-
C02:15
HLA-
C02:16
HLA-
C02:17
HLA-
C02:18
HLA-
C02:19
HLA-
C02:20
HLA-
C02:21
HLA-
C02:22
HLA-
C02:23
HLA-
C02:24
HLA-
C02:26
HLA-
C02:27
HLA-
C02:28
HLA-
C02:29
HLA-
C02:30
HLA-
C02:31
HLA-
C02:32
HLA-
C02:33
HLA-
C02:34
HLA-
C02:35
HLA-
C02:36
HLA-
C02:37
HLA-
C02:39
HLA-
C02:40
HLA-
C03:01
HLA-
C03:02
HLA-
C03:03
HLA-
C03:04
HLA-
C03:05
HLA-
C03:06
HLA-
C03:07
HLA-
C03:08
HLA-
C03:09
HLA-
C03:10
HLA-
C03:11
HLA-
C03:12
HLA-
C03:13
HLA-
C03:14
HLA-
C03:15
HLA-
C03:16
HLA-
C03:17
HLA-
C03:18
HLA-
C03:19
HLA-
C03:21
HLA-
C03:23
HLA-
C03:24
HLA-
C03:25
HLA-
C03:26
HLA-
C03:27
HLA-
C03:28
HLA-
C03:29
HLA-
C03:30
HLA-
C03:31
HLA-
C03:32
HLA-
C03:33
HLA-
C03:34
HLA-
C03:35
HLA-
C03:36
HLA-
C03:37
HLA-
C03:38
HLA-
C03:39
HLA-
C03:40
HLA-
C03:41
HLA-
C03:42
HLA-
C03:43
HLA-
C03:44
HLA-
C03:45
HLA-
C03:46
HLA-
C03:47
HLA-
C03:48
HLA-
C03:49
HLA-
C03:50
HLA-
C03:51
HLA-
C03:52
HLA-
C03:53
HLA-
C03:54
HLA-
C03:55
HLA-
C03:56
HLA-
C03:57
HLA-
C03:58
HLA-
C03:59
HLA-
C03:60
HLA-
C03:61
HLA-
C03:62
HLA-
C03:63
HLA-
C03:64
HLA-
C03:65
HLA-
C03:66
HLA-
C03:67
HLA-
C03:68
HLA-
C03:69
HLA-
C03:70
HLA-
C03:71
HLA-
C03:72
HLA-
C03:73
HLA-
C03:74
HLA-
C03:75
HLA-
C03:76
HLA-
C03:77
HLA-
C03:78
HLA-
C03:79
HLA-
C03:80
HLA-
C03:81
HLA-
C03:82
HLA-
C03:83
HLA-
C03:84
HLA-
C03:85
HLA-
C03:86
HLA-
C03:87
HLA-
C03:88
HLA-
C03:89
HLA-
C03:90
HLA-
C03:91
HLA-
C03:92
HLA-
C03:93
HLA-
C03:94
HLA-
C04:01
HLA-
C04:03
HLA-
C04:04
HLA-
C04:05
HLA-
C04:06
HLA-
C04:07
HLA-
C04:08
HLA-
C04:10
HLA-
C04:11
HLA-
C04:12
HLA-
C04:13
HLA-
C04:14
HLA-
C04:15
HLA-
C04:16
HLA-
C04:17
HLA-
C04:18
HLA-
C04:19
HLA-
C04:20
HLA-
C04:23
HLA-
C04:24
HLA-
C04:25
HLA-
C04:26
HLA-
C04:27
HLA-
C04:28
HLA-
C04:29
HLA-
C04:30
HLA-
C04:31
HLA-
C04:32
HLA-
C04:33
HLA-
C04:34
HLA-
C04:35
HLA-
C04:36
HLA-
C04:37
HLA-
C04:38
HLA-
C04:39
HLA-
C04:40
HLA-
C04:41
HLA-
C04:42
HLA-
C04:43
HLA-
C04:44
HLA-
C04:45
HLA-
C04:46
HLA-
C04:47
HLA-
C04:48
HLA-
C04:49
HLA-
C04:50
HLA-
C04:51
HLA-
C04:52
HLA-
C04:53
HLA-
C04:54
HLA-
C04:55
HLA-
C04:56
HLA-
C04:57
HLA-
C04:58
HLA-
C04:60
HLA-
C04:61
HLA-
C04:62
HLA-
C04:63
HLA-
C04:64
HLA-
C04:65
HLA-
C04:66
HLA-
C04:67
HLA-
C04:68
HLA-
C04:69
HLA-
C04:70
HLA-
C05:01
HLA-
C05:03
HLA-
C05:04
HLA-
C05:05
HLA-
C05:06
HLA-
C05:08
HLA-
C05:09
HLA-
C05:10
HLA-
C05:11
HLA-
C05:12
HLA-
C05:13
HLA-
C05:14
HLA-
C05:15
HLA-
C05:16
HLA-
C05:17
HLA-
C05:18
HLA-
C05:19
HLA-
C05:20
HLA-
C05:21
HLA-
C05:22
HLA-
C05:23
HLA-
C05:24
HLA-
C05:25
HLA-
C05:26
HLA-
C05:27
HLA-
C05:28
HLA-
C05:29
HLA-
C05:30
HLA-
C05:31
HLA-
C05:32
HLA-
C05:33
HLA-
C05:34
HLA-
C05:35
HLA-
C05:36
HLA-
C05:37
HLA-
C05:38
HLA-
C05:39
HLA-
C05:40
HLA-
C05:41
HLA-
C05:42
HLA-
C05:43
HLA-
C05:44
HLA-
C05:45
HLA-
C06:02
HLA-
C06:03
HLA-
C06:04
HLA-
C06:05
HLA-
C06:06
HLA-
C06:07
HLA-
C06:08
HLA-
C06:09
HLA-
C06:10
HLA-
C06:11
HLA-
C06:12
HLA-
C06:13
HLA-
C06:14
HLA-
C06:15
HLA-
C06:17
HLA-
C06:18
HLA-
C06:19
HLA-
C06:20
HLA-
C06:21
HLA-
C06:22
HLA-
C06:23
HLA-
C06:24
HLA-
C06:25
HLA-
C06:26
HLA-
C06:27
HLA-
C06:28
HLA-
C06:29
HLA-
C06:30
HLA-
C06:31
HLA-
C06:32
HLA-
C06:33
HLA-
C06:34
HLA-
C06:35
HLA-
C06:36
HLA-
C06:37
HLA-
C06:38
HLA-
C06:39
HLA-
C06:40
HLA-
C06:41
HLA-
C06:42
HLA-
C06:43
HLA-
C06:44
HLA-
C06:45
HLA-
C07:01
HLA-
C07:02
HLA-
C07:03
HLA-
C07:04
HLA-
C07:05
HLA-
C07:06
HLA-
C07:07
HLA-
C07:08
HLA-
C07:09
HLA-
C07:10
HLA-
C07:11
HLA-
C07:12
HLA-
C07:13
HLA-
C07:14
HLA-
C07:15
HLA-
C07:16
HLA-
C07:17
HLA-
C07:18
HLA-
C07:19
HLA-
C07:20
HLA-
C07:21
HLA-
C07:22
HLA-
C07:23
HLA-
C07:24
HLA-
C07:25
HLA-
C07:26
HLA-
C07:27
HLA-
C07:28
HLA-
C07:29
HLA-
C07:30
HLA-
C07:31
HLA-
C07:35
HLA-
C07:36
HLA-
C07:37
HLA-
C07:38
HLA-
C07:39
HLA-
C07:40
HLA-
C07:41
HLA-
C07:42
HLA-
C07:43
HLA-
C07:44
HLA-
C07:45
HLA-
C07:46
HLA-
C07:47
HLA-
C07:48
HLA-
C07:49
HLA-
C07:50
HLA-
C07:51
HLA-
C07:52
HLA-
C07:53
HLA-
C07:54
HLA-
C07:56
HLA-
C07:57
HLA-
C07:58
HLA-
C07:59
HLA-
C07:60
HLA-
C07:62
HLA-
C07:63
HLA-
C07:64
HLA-
C07:65
HLA-
C07:66
HLA-
C07:67
HLA-
C07:68
HLA-
C07:69
HLA-
C07:70
HLA-
C07:71
HLA-
C07:72
HLA-
C07:73
HLA-
C07:74
HLA-
C07:75
HLA-
C07:76
HLA-
C07:77
HLA-
C07:78
HLA-
C07:79
HLA-
C07:80
HLA-
C07:81
HLA-
C07:82
HLA-
C07:83
HLA-
C07:84
HLA-
C07:85
HLA-
C07:86
HLA-
C07:87
HLA-
C07:88
HLA-
C07:89
HLA-
C07:90
HLA-
C07:91
HLA-
C07:92
HLA-
C07:93
HLA-
C07:94
HLA-
C07:95
HLA-
C07:96
HLA-
C07:97
HLA-
C07:99
HLA-
C07:100
HLA-
C07:101
HLA-
C07:102
HLA-
C07:103
HLA-
C07:105
HLA-
C07:106
HLA-
C07:107
HLA-
C07:108
HLA-
C07:109
HLA-
C07:110
HLA-
C07:111
HLA-
C07:112
HLA-
C07:113
HLA-
C07:114
HLA-
C07:115
HLA-
C07:116
HLA-
C07:117
HLA-
C07:118
HLA-
C07:119
HLA-
C07:120
HLA-
C07:122
HLA-
C07:123
HLA-
C07:124
HLA-
C07:125
HLA-
C07:126
HLA-
C07:127
HLA-
C07:128
HLA-
C07:129
HLA-
C07:130
HLA-
C07:131
HLA-
C07:132
HLA-
C07:133
HLA-
C07:134
HLA-
C07:135
HLA-
C07:136
HLA-
C07:137
HLA-
C07:138
HLA-
C07:139
HLA-
C07:140
HLA-
C07:141
HLA-
C07:142
HLA-
C07:143
HLA-
C07:144
HLA-
C07:145
HLA-
C07:146
HLA-
C07:147
HLA-
C07:148
HLA-
C07:149
HLA-
C08:01
HLA-
C08:02
HLA-
C08:03
HLA-
C08:04
HLA-
C08:05
HLA-
C08:06
HLA-
C08:07
HLA-
C08:08
HLA-
C08:09
HLA-
C08:10
HLA-
C08:11
HLA-
C08:12
HLA-
C08:13
HLA-
C08:14
HLA-
C08:15
HLA-
C08:16
HLA-
C08:17
HLA-
C08:18
HLA-
C08:19
HLA-
C08:20
HLA-
C08:21
HLA-
C08:22
HLA-
C08:23
HLA-
C08:24
HLA-
C08:25
HLA-
C08:27
HLA-
C08:28
HLA-
C08:29
HLA-
C08:30
HLA-
C08:31
HLA-
C08:32
HLA-
C08:33
HLA-
C08:34
HLA-
C08:35
HLA-
C12:02
HLA-
C12:03
HLA-
C12:04
HLA-
C12:05
HLA-
C12:06
HLA-
C12:07
HLA-
C12:08
HLA-
C12:09
HLA-
C12:10
HLA-
C12:11
HLA-
C12:12
HLA-
C12:13
HLA-
C12:14
HLA-
C12:15
HLA-
C12:16
HLA-
C12:17
HLA-
C12:18
HLA-
C12:19
HLA-
C12:20
HLA-
C12:21
HLA-
C12:22
HLA-
C12:23
HLA-
C12:24
HLA-
C12:25
HLA-
C12:26
HLA-
C12:27
HLA-
C12:28
HLA-
C12:29
HLA-
C12:30
HLA-
C12:31
HLA-
C12:32
HLA-
C12:33
HLA-
C12:34
HLA-
C12:35
HLA-
C12:36
HLA-
C12:37
HLA-
C12:38
HLA-
C12:40
HLA-
C12:41
HLA-
C12:43
HLA-
C12:44
HLA-
C14:02
HLA-
C14:03
HLA-
C14:04
HLA-
C14:05
HLA-
C14:06
HLA-
C14:08
HLA-
C14:09
HLA-
C14:10
HLA-
C14:11
HLA-
C14:12
HLA-
C14:13
HLA-
C14:14
HLA-
C14:15
HLA-
C14:16
HLA-
C14:17
HLA-
C14:18
HLA-
C14:19
HLA-
C14:20
HLA-
C15:02
HLA-
C15:03
HLA-
C15:04
HLA-
C15:05
HLA-
C15:06
HLA-
C15:07
HLA-
C15:08
HLA-
C15:09
HLA-
C15:10
HLA-
C15:11
HLA-
C15:12
HLA-
C15:13
HLA-
C15:15
HLA-
C15:16
HLA-
C15:17
HLA-
C15:18
HLA-
C15:19
HLA-
C15:20
HLA-
C15:21
HLA-
C15:22
HLA-
C15:23
HLA-
C15:24
HLA-
C15:25
HLA-
C15:26
HLA-
C15:27
HLA-
C15:28
HLA-
C15:29
HLA-
C15:30
HLA-
C15:31
HLA-
C15:33
HLA-
C15:34
HLA-
C15:35
HLA-
C16:01
HLA-
C16:02
HLA-
C16:04
HLA-
C16:06
HLA-
C16:07
HLA-
C16:08
HLA-
C16:09
HLA-
C16:10
HLA-
C16:11
HLA-
C16:12
HLA-
C16:13
HLA-
C16:14
HLA-
C16:15
HLA-
C16:17
HLA-
C16:18
HLA-
C16:19
HLA-
C16:20
HLA-
C16:21
HLA-
C16:22
HLA-
C16:23
HLA-
C16:24
HLA-
C16:25
HLA-
C16:26
HLA-
C17:01
HLA-
C17:02
HLA-
C17:03
HLA-
C17:04
HLA-
C17:05
HLA-
C17:06
HLA-
C17:07
HLA-
C18:01
HLA-
C18:02
HLA-
C18:03

TABLE 7
List of HLA-Class II subtypes against which binding
affinity of peptides are calculated

| HLA DR | HLA DQ | HLA DP |
|---|---|---|
| HLA-DRB1*01:01 | HLA-DQA1*05:01/DQB1*02:01 | HLA-DPA1*02:01/DPB1*01:01 |
| HLA-DRB1*03:01 | HLA-DQA1*05:01/DQB1*03:01 | HLA-DPA1*01:03/DPB1*02:01 |
| HLA-DRB1*04:01 | HLA-DQA1*03:01/DQB1*03:02 | HLA-DPA1*01/DPB1*04:01 |
| HLA-DRB1*04:05 | HLA-DQA1*04:01/DQB1*04:02 | HLA-DPA1*03:01/DPB1*04:02 |
| HLA-DRB1*07:01 | HLA-DQA1*01:01/DQB1*05:01 | HLA-DPA1*02:01/DPB1*05:01 |
| HLA-DRB1*08:02 | HLA-DQA1*01:02/DQB1*06:02 | HLA-DPA1*02:01/DPB1*14:01 |
| HLA-DRB1*09:01 | | |
| HLA-DRB1*11:01 | | |
| HLA-DRB1*12:01 | | |
| HLA-DRB1*13:02 | | |
| HLA-DRB1*15:01 | | |
| HLA-DRB3*01:01 | | |
| HLA-DRB3*02:02 | | |
| HLA-DRB4*01:01 | | |
| HLA-DRB5*01:01 | | |

TABLE 8
Peptides classified as Non-immunogenic in the
IEDB database used for developing the TCR-
binding algorithm

| | | | | |
|---|---|---|---|---|
| WLLIDTSNA | SLAGFVRML | KLDKEMEAV | DVVNGLANL | VLLLDVTPL |
| RVSRPTTVV | GLFLTTEAV | VLADANETL | ALAPAPVEV | AIYHPQQFV |
| YLDLALMSV | RLQSLQTYV | MLGNAPSVV | YLGKLFVTL | AMKADIQHV |
| FIFLLFLTL | LLPLGYPFV | LLWQDPVPA | GADEDDIKA | ALLSDWLPA |
| DETGVEVKD | ALLRQLAEL | RLLEAFQFV | KLLTKPWDV | RMFAANLGV |
| LMLPGMNGI | FVVALIPLV | LLPPELSET | WMHHNMDLV | MLQDMAILT |
| EMKEGRYEV | SLQNSEFLL | GLVDFVKHI | GLYLSQIAV | ALLWAAGVL |
| VLLEKATIL | AYGSFVRTV | VLLEQMGSL | ILFTFLHLA | LLFRFMRPL |
| SLLERGQQL | GLMTAVYLV | MLADKTKSI | TEVGQDQYV | RLGAVILFV |
| YLSEGDMAA | LMHAPAFET | LVLEQLGQL | TRHPATATV | DLSRDLDSV |
| RVYEALYYV | GLYYLTTEV | RMPAVTDLV | LLFLGVVFL | GLYGAQYDV |
| KLGLLQVTG | LLYNEQFAV | TRVTIWKSK | ILSSLGLPV | FLAVGGVLL |
| SMAGNWAKV | VVFEDVKGT | YLSQIAVLL | FANYNFTLL | MLASTLTDA |
| VVWVKITQV | ALSTGLIHL | YLLALRYLA | ILLSIARVV | YLVTSINKL |
| GLYRQWALA | FIPENQRTV | RLMIGTAAA | IVYEAADAI | SLPKHNVTI |
| SMGIFLKSL | DLPSGFNTL | FLLPDAQSI | KFRVQGEAV | RLARAIIEL |
| SLFPEFSEL | GLFGKGSLV | YTYKWETFL | RLLDDTPEV | MALLRLPLV |
| GESVPGIEE | NSNDIVNAI | AETGSGTAS | KIFCISIFL | YKSPASDAY |
| YLYVHSPAL | TVLRFVPPL | KLCTFSFLI | AMLQDMAIL | KLSSFFQSV |
| FMKAVCVEV | SLLEIGEGV | FLIHSADWL | ALVLLMLPV | AIMDKKIIL |
| DSTQTTTQK | VIADYNYKL | ALWGPDPAA | MIAAYTAAL | |
| YSLEYFQFV | AIMDKTVIL | NILFVITKL | AALGLWLSV | |
| RAKAVRALK | KVLTLFAEV | LLACAVIHA | VLCPYMPKV | |
| TEQELPQSQ | SRAKAVRAL | TLAARIKFL | ALIIIRSLL | |

TABLE 9
Peptides classified as Immunogenic in the
IEDB database used for developing the TCR-
binding algorithm

| | | | | |
|---|---|---|---|---|
| SLKDVLVSV | LLMWEAVTV | ILLWEIPDV | FLYGALLLA | MINPLVITT |
| VAALFFFDI | GMLGFVFTL | HLMIDRPYV | LLDVAPLSL | FILPVLGAV |
| SLWGGDVVL | LGYGFVNYI | ALISAFSGS | MGLPGVATV | FAFRDLCIV |
| AMDTISVFL | LIVDAVLQL | RQYDPVAAL | FANCNFTLV | RMFPNAPYL |
| VLLLWITAA | FLLDILGAT | LLIGGFAGL | NLNESLIDL | KVLIRCYLC |
| MLWYTVYNI | RLLQTGIHV | FANYKFTLV | LLWSYAMGV | KLIVTPAAL |
| RVPGVAPTL | FLGERVTLT | VPILLKALY | FMVFLQTHI | VLQELNVTV |
| WLDEVKQAL | FVNYDFTIV | LLWNGPMAV | RVNRLIIWV | LLNYILKSV |
| ALNTPKDHI | KLNDWDFVV | KLSDYEGRL | SLMSGVEPL | MMFGFHHSV |
| AWLVAAAEI | LFLNTLSFV | GMVTTSTTL | TLDYKPLSV | IVLGLIATA |
| AILHTPGCV | GGNGMLATI | SLVEELKKV | SLFNTVATL | YLNKIQNSL |
| GLLDQVAAL | SFHSLHLLF | ALSALLTKL | VLLRHSKNV | QLLSSSKYT |
| SQQAQLAAA | AIIIAVLLV | FVDYNFSLV | VLLCVCLLI | CLFKDWEEL |
| TLKDIVLDL | RFIAQLLLL | ILLNKHIDA | SLLMWITQC | AIIDPLIYA |
| MLNIPSINV | SIYVYALPL | ILNNPKASL | GLNDYLHSV | TLGIVCPIC |
| AIMDKVIIL | PTLDKVLEV | FQQLFLNTL | AMASTEGNV | KYQEFFWDA |
| GILGFVYTL | LVLILYLCV | ALLGLTLGV | GLREDLLSL | LALPMPATA |
| TLEEFSAKL | GMSRIGMEV | GLMWLSYFV | KLWCRHFCV | SLMSWSAIL |
| LLDAHIPQL | FLSHDFTLV | KVDDTFYYV | ALAIIIAVL | KVLGLWATV |
| RTLDKVLEV | CINGVCWSV | ALFHEVAKL | LQLPQGTTL | ILPDPLKPT |
| YLESFCEDV | SITEVECFL | SLPRSRTPI | FLWEDQTLL | FLSFASLFL |
| RMTENIVEV | RLERKWLDV | LMLIWYRPV | FLLKLTPLL | ILIEGVFFA |
| GILGVVFTL | SIDQLCKTF | IVIEAIHTV | GIWGFVFTL | ALLEDPVGT |
| RGTPMVITV | QLFNHTMFI | SLILVSQYT | FANHKFTLV | FVNYNFTLV |
| HLGNVKYLV | MIMQGGFSV | GTLGFVFTL | QMMRNEFRV | WQWEHIPPA |
| VVPEDYWGV | KCIDFYSRI | RLNEVAKNL | FLLCFCVLL | VMLFILAGL |
| VLNDILSRL | SLKKNSRSL | MINAYLDKL | GILTVSVAV | MTYAAPLFV |
| RLPLVLPAV | MLDLQPETT | TIDQLCKTF | FVDYNFTIV | YLKKIKNSL |
| VLNETTNWL | MTIIFLILM | LVLPILITI | ALYDVVSKL | AMAGASTSA |
| ALSEDLLSI | NDFCCVATV | AIVDKNITL | LFAAFPSFA | NMLSTVLGV |
| GILGFIFTL | YLEPGPVTA | RLIQNSITI | LLGRNSFEV | ILAKFLHWL |
| IMVLSFLFL | ILDKKVEKV | ILRSFIPLL | MLLDKNIPI | KLGPGEEQV |
| TLAPQVEPL | LALLLLDRL | FANHNFTLV | MLWGYLQYV | SVYDFFVWL |
| FTWEGLYNV | FIDKFTPPV | QLSTRGVQI | NLLTTPKFT | VLTSESMHV |
| AIMDKTIIL | GILEFVFTL | LLSILCIWV | TLYAVATTI | SLSRFSWGA |
| GVLGFVFTL | YLVSIFLHL | PTLDKVLEL | FLKQQYMNL | RMLGDVMAV |
| RLQGISPKI | FVVPILLKA | GVRVLEDGV | KDLVLLATI | YILEETSVM |
| ALLKDTVYT | CLPACVYGL | MVMELIRMI | LLVSEIDWL | ILDAHSLYL |
| LLLIWFRPV | VLSEWLPVT | SAPLPSNRV | KLNPMLAKA | GIFEDRAPV |
| VAANIVLTV | TLLDHIRTA | LQLCCLATA | VIFDFLHCI | TVCGGIMFL |
| AMLHWSLIL | KMLKEMGEV | ELTEVFEFA | FANNEFTLV | GLCPHCINV |
| ALAVLSVTL | AVADHVAAV | CLTEYILWV | VLCLRPVGA | AFLGERVTL |
| SGDGLVATG | TLNDLETDV | YLIIGILTL | SLFLGILSV | NGVRVLATA |
| GLSISGNLL | TLLANVTAV | GILGLVFTL | ALAHGVRAL | QLLNSVLTL |
| YLLPAIVHI | SLVNGVVRL | AMLNGLIYV | ALLALTRAI | ILHTNMPNV |
| WILGFVFTL | ALPHIIDEV | RMLPHAPGV | NLLIRCLRC | AITEVECFL |
| SLSAYIIRV | LITGRLQSL | LLIDLTSFL | SMINGVVKL | GMDPRMCSL |
| KLVCSPAPC | TLTSYWRRV | LLLGTLNIV | DVSRPTAVV | AILIRVRNA |
| YINTALLNA | FQGRGVFEL | FANYNFTLV | ALNTLVKQL | KTVLELTEV |
| ILLARLFLY | SLMDLLSSL | LGYGFVNYV | FIAGLIAIV | VLHKRTLGL |
| YLDKVRATV | FLTSVINRV | TLACFAVYT | KTWGQYWQV | MGNGCLRIV |
| FANNKFTLV | GILDFGVKL | SLNQTVHSL | RMSKGVFKV | LVMAQLLRI |
| LLHTDFEQV | QLVQSGAEV | RLNTVLATA | ILYGPLTRI | AMLDLLKSV |
| FLYELIWNV | YLLKPVQRI | IVSPFIPLL | HLSLRGLPV | IADAALAAL |
| LLCGNLLIL | SLPITVYYA | LLIEGIFFI | SLFGGMSWI | DLSLRRFMV |
| LIDQYLYYL | AIMDKNITL | SIVAYTMSL | LLLLDVAPL | LQDIEITCV |
| LLYNCCYHV | RINAILATA | ELLRPTTLV | FLMEDQTLL | KLQEQQSDL |
| RDVPMLITT | FVNHRFTLV | FAFKDLFVV | AMDSNTLEL | FLTCTDRSV |
| PESSQRPPL | LLSLFSLWL | NIVCPLCTL | ITNCLLSTA | SVGGVFTSV |
| LMGDKSENV | ALAEGDLLA | GGPNLDNIL | ILIEGIFFA | |
| RLNELLAYV | TLARGFPFV | TIPEALAAV | DLMGYIPAV | |
| RLWHYPCTI | LIFLARSAL | TLLYVLFEV | AMLVLLAEI | |

TABLE 10
Performance metrices for the different classifiers on unseen dataset

| | HLA binding | Ensemble | Ensemble | Ensemble |
|---|---|---|---|---|
| Performance metric | classifier | classifier1 | classifier2 | classifier3 |
| TP | 228 | 183 | 220 | 277 |
| FP | 84 | 44 | 9 | 1 |
| TN | 32 | 72 | 107 | 115 |
| FN | 78 | 124 | 87 | 30 |
| Sensitivity (%) | 74.50% | 59.61 | 71.66 | 90.23 |
| Specificity (%) | 27.59% | 62.07 | 92.24 | 99.14 |
| Accuracy (%) | 61.61% | 60.28 | 77.30 | 92.67 |

TP: True Positive (Immunogenic peptide predicted as immunogenic)
FP: False Positive (Non-immunogenic peptide predicted as immunogenic)
TN: True Negative (Non-immunogenic peptide predicted as non-immunogenic)
FN: False Negative (Non-immunogenic peptide predicted as immunogenic)
HLA binding classifier: If the peptide binding affinity using NetMHCcons program is <=500 nM then it is taken as immunogenic peptide and rest other as non-immunogenic peptide
Ensemble classifier1: The ensemble J4.8 classifier built using 500 classifiers using all features for the peptides.
Ensemble classifier2: The ensemble J4.8 classifier built using 433 classifiers using reduced features for the peptides.
Ensemble classifier3: The ensemble J4.8 classifier built using 45 best individual classifiers using reduced features for the peptides.

TABLE 11
List of selected features defining hydrophobicity and helix/turn and
their position in peptide and their frequency in immunogenic peptides

| Frequency | Position in 9mer | Feature ID^{1,2} | Feature Type | Brief description |
|---|---|---|---|---|
| 12 | 8, 9 | RACS820104 | helix/turn | Average relative |
| | | | | fractional occurrence |
| | | | | in EL |
| 7 | 8, 9 | JOND750102 | hydrophobicity | pK (—COOH) |
| 7 | 3 | TANS770108 | helix/turn | Normalized |
| | | | | frequency of zeta R |
| 7 | 4, 5 | RICJ880115 | helix/turn | Relative preference |
| | | | | value at C-cap |
| 6 | 5, 6 | RICJ880109 | helix/turn | Relative preference |
| | | | | value at Mid |
| 6 | 6 | PALJ810109 | helix/turn | Normalized |
| | | | | frequency of alpha- |
| | | | | helix in alpha/beta |
| | | | | class |
| 5 | 1, 2, 3, 4, 5, 6, 7, 8, 9 | NAKH920106 | helix/turn | AA composition of |
| | | | | CYT of multi- |
| | | | | spanning proteins |
| 4 | 2 | MEEJ800102 | hydrophobicity | Retention coefficient |
| | | | | in HPLC |
| 4 | 8, 9 | CEDJ970101 | hydrophobicity | Composition of amino |
| | | | | acids in extracellular |
| | | | | proteins |
| 4 | 1, 2, 3, 4, 5, 6, 7, 8, 9 | WILM950103 | hydrophobicity | Hydrophobicity |
| | | | | coefficient in RP- |
| | | | | HPLC |
| 4 | 2, 3 | RICJ880104 | helix/turn | Relative preference |
| | | | | value at N1 |
| 4 | 7, 8 | QIAN880137 | helix/turn | Weights for coil at the |
| | | | | window position of 4 |
| 4 | 8, 9 | PALJ810108 | helix/turn | Normalized |
| | | | | frequency of alpha- |
| | | | | helix in alpha + beta |
| | | | | class |
| 4 | 1, 2, 8, 9 | QIAN880127 | helix/turn | Weights for coil at the |
| | | | | window position of −6 |
| 4 | 3, 4, 5, 6, 7, 8 | SUYM030101 | helix/turn | Linker propensity |
| | | | | index |
| 3 | 2, 3 | WILM950104 | hydrophobicity | Hydrophobicity |
| | | | | coefficient in RP- |
| | | | | HPLC |
| 3 | 3 | WILM950103 | hydrophobicity | Hydrophobicity |
| | | | | coefficient in RP- |
| | | | | HPLC |
| 3 | 1, 2, 3, 4, 5, 6, 7, 8, 9 | WILM950104 | hydrophobicity | Hydrophobicity |
| | | | | coefficient in RP- |
| | | | | HPLC |
| 3 | 1, 2, 3, 4, 5, 6, 7, 8, 9 | NAKH900108 | hydrophobicity | Normalized |
| | | | | composition from |
| | | | | fungi and plant |
| 3 | 1, 2 | RACS820107 | helix/turn | Average relative |
| | | | | fractional occurrence |
| | | | | in A0 |
| 3 | 1, 2 | ROBB760111 | helix/turn | Information measure |
| | | | | for C-terminal turn |
| 3 | 1, 2 | TANS770102 | helix/turn | Normalized |
| | | | | frequency of isolated |
| | | | | helix |
| 3 | 1, 2 | QIAN880139 | helix/turn | Weights for coil at the |
| | | | | window position of 6 |
| 3 | 2, 3 | RICJ880113 | helix/turn | Relative preference |
| | | | | value at C2 |
| 3 | 5, 6 | RICJ880105 | helix/turn | Relative preference |
| | | | | value at N2 |
| 3 | 6 | CHOP780204 | helix/turn | Normalized |
| | | | | frequency of N- |
| | | | | terminal helix |
| 3 | 6, 7 | PALJ810108 | helix/turn | Normalized |
| | | | | frequency of alpha- |
| | | | | helix in alpha + beta |
| | | | | class |
| 3 | 6, 7 | PALJ810113 | helix/turn | Normalized |
| | | | | frequency of turn in |
| | | | | all-alpha class |
| 3 | 3, 4, 5, 6, 7, 8 | RACS820107 | helix/turn | Average relative |
| | | | | fractional occurrence |
| | | | | in A0 |
| 3 | 3, 4, 5, 6, 7, 8 | RICJ880110 | helix/turn | Relative preference |
| | | | | value at C5 |
| 3 | 1, 2, 3, 4, 5, 6, 7, 8, 9 | SUYM030101 | helix/turn | Linker propensity |
| | | | | index |
| 2 | 1, 2, 3, 4, 5, 6, 7, 8, 9 | XLogP.VAR | hydrophobicity | An estimate of the |
| | | | | logP partition |
| | | | | coefficient |
| 2 | 2, 3 | KIDA850101 | hydrophobicity | Hydrophobicity- |
| | | | | related index |
| 2 | 3 | RADA880101 | hydrophobicity | Transfer free energy |
| | | | | from chx to wat |
| 2 | 3 | RADA880104 | hydrophobicity | Transfer free energy |
| | | | | from chx to oct |
| 2 | 3 | WILM950104 | hydrophobicity | Hydrophobicity |
| | | | | coefficient in RP- |
| | | | | HPLC |
| 2 | 5, 6 | BULH740102 | hydrophobicity | Apparent partial |
| | | | | specific volume |
| 2 | 6 | CIDH920103 | hydrophobicity | Normalized |
| | | | | hydrophobicity scales |
| | | | | for alpha + beta- |
| | | | | proteins |
| 2 | 6, 7 | RADA880107 | hydrophobicity | Energy transfer from |
| | | | | out to in(95% buried) |
| 2 | 6, 7 | PONP800103 | hydrophobicity | Average gain ratio in |
| | | | | surrounding |
| | | | | hydrophobicity |
| 2 | 1, 2, 8, 9 | KANM800104 | hydrophobicity | Average relative |
| | | | | probability of inner |
| | | | | beta-sheet |
| 2 | 1, 2, 3, 4, 5, 6, 7, 8, 9 | ZASB820101 | hydrophobicity | Dependence of |
| | | | | partition coefficient |
| | | | | on ionic strength |
| 2 | 1 | SUEM840102 | helix/turn | Zimm-Bragg |
| | | | | parameter sigma x |
| | | | | 1.0E4 |
| 2 | 1, 2 | PALJ810108 | helix/turn | Normalized |
| | | | | frequency of alpha- |
| | | | | helix in alpha + beta |
| | | | | class |
| 2 | 1, 2 | LEVM780104 | helix/turn | Normalized |
| | | | | frequency of alpha- |
| | | | | helix |
| 2 | 1, 2 | RICJ880104 | helix/turn | Relative preference |
| | | | | value at N1 |
| 2 | 2 | GEIM800109 | helix/turn | Aperiodic indices for |
| | | | | alpha-proteins |
| 2 | 2 | ROBB760111 | helix/turn | Information measure |
| | | | | for C-terminal turn |
| 2 | 2 | QIAN880112 | helix/turn | Weights for alpha- |
| | | | | helix at the window |
| | | | | position of 5 |
| 2 | 2, 3 | CHOP780212 | helix/turn | Frequency of the 1st |
| | | | | residue in turn |
| 2 | 2, 3 | BUNA790101 | helix/turn | alpha-NH chemical |
| | | | | shifts |
| 2 | 2, 3 | RICJ880114 | helix/turn | Relative preference |
| | | | | value at C1 |
| 2 | 3 | RACS820103 | helix/turn | Average relative |
| | | | | fractional occurrence |
| | | | | in AL |
| 2 | 3, 4 | RICJ880109 | helix/turn | Relative preference |
| | | | | value at Mid |
| 2 | 4, 5 | RICJ880113 | helix/turn | Relative preference |
| | | | | value at C2 |
| 2 | 5, 6 | RACS820105 | helix/turn | Average relative |
| | | | | fractional occurrence |
| | | | | in E0 |
| 2 | 6 | CHOP780213 | helix/turn | Frequency of the 2nd |
| | | | | residue in turn |
| 2 | 6 | RACS820106 | helix/turn | Average relative |
| | | | | fractional occurrence |
| | | | | in ER |
| 2 | 6 | PALJ810107 | helix/turn | Normalized |
| | | | | frequency of alpha- |
| | | | | helix in all-alpha |
| | | | | class |
| 2 | 6 | QIAN880106 | helix/turn | Weights for alpha- |
| | | | | helix at the window |
| | | | | position of −1 |
| 2 | 6, 7 | MAXF760103 | helix/turn | Normalized |
| | | | | frequency of zeta R |
| 2 | 6, 7 | QIAN880137 | helix/turn | Weights for coil at the |
| | | | | window position of 4 |
| 2 | 7, 8 | QIAN880101 | helix/turn | Weights for alpha- |
| | | | | helix at the window |
| | | | | position of −6 |
| 2 | 8, 9 | QIAN880102 | helix/turn | Weights for alpha- |
| | | | | helix at the window |
| | | | | position of −5 |
| 2 | 8, 9 | NAKH920101 | helix/turn | AA composition of |
| | | | | CYT of single- |
| | | | | spanning proteins |
| 2 | 3, 4, 5, 6, 7, 8 | RICJ880109 | helix/turn | Relative preference |
| | | | | value at Mid |

¹Amino acid index
²PepLib library ID

### Example 1a

A method of selecting immunogenic peptide from a peptide sequence
TCR binding prediction
Features of amino acids at each of the 9 positions of the 9-mer peptide considered for predicting immunogenicity

| Feature | | | |
|---|---|---|---|
| number | Feature value | Feature ID | Feature description |
| f1 | Average value of | RICJ880105¹ | Relative preference value at N2 |
| | position 5, 6 | | (Richardson-Richardson) |
| f2 | Average value of | QIAN880107¹ | Weights for alpha-helix at the |
| | position 1, 2, 8, 9 | | window position of 0 (Qian- |
| | | | Sejnowski) |
| f3 | Average value of | YUTK870103¹ | Activation Gibbs energy of unfolding |
| | position 8, 9 | | |
| f4 | Value of position 3 | FNSA.2² | a combination of surface area and |
| | | | partial charge |
| f5 | Average value of | VASM830101¹ | Relative population of |
| | position 6, 7 | | conformational state A (Vasquez et |
| | | | al.) |
| f6 | Average value of | ROBB760108¹ | Information measure for turn |
| | position 6, 7 | | (Robson-Suzuki) |
| f7 | Average value of | NAKH920106¹ | AA composition of CYT of multi- |
| | position 1-9 | | spanning proteins (Nakashima- |
| | | | Nishikawa) |
| f8 | Average value of | QIAN880139¹ | Weights for coil at the window |
| | position 2, 3 | | position of 6 (Qian-Sejnowski) |
| f9 | Average value of | QIAN880138¹ | Weights for coil at the window |
| | position 7, 8 | | position of 5 (Qian-Sejnowski) |
| f10 | Average value of | CHAM830103¹ | The number of atoms in the side |
| | position 1-9 | | chain labelled 1 + 1 (Charton- |
| | | | Charton) |
| f11 | Average value of | YUTK870103¹ | Activation Gibbs energy of unfolding |
| | position 5, 6 | | |
| f12 | Average value of | MITS020101¹ | Amphiphilicity index (Mitaku et al.) |
| | position 1, 2 | | |
| f13 | Value of position 2 | PNSA.1.AUTO² | a combination of surface area and |
| | | | partial charge |
| f14 | Value of position 3 | KARS160118¹ | Average weighted atomic number or |
| | | | degree based on atomic number in |
| | | | the graph (Karkbara-Knisley) |
| f15 | Average value of | YUTK870104¹ | Activation Gibbs energy of unfolding |
| | position 8, 9 | | |

Rules for predicting immunogenicity based on the features of amino acids at each of the 9 positions of the 9-mer peptide. The rules specify the range of parameters that define the identity of each amino acid at each position of the 9-mer peptide

Rule 1: f1<=0.5

Rule 2: f1>0.5 AND f2<=−0.77

Rule 3: f1>0.5 AND f2>−0.77 AND f3<=17.75

Rule 4: f1>0.5 AND f2>−0.77 AND f3>17.75 AND f4<=−0.34 AND f5<=0.2055

Rule 5: f1>0.5 AND f2>−0.77 AND f3>17.75 AND f4>−0.34 AND f6<=−5.5

Rule 6: f1>0.5 AND f2>−0.77 AND f3>17 75 AND f4>−0.34 AND f6>−5.5 AND f7<=45.56 AND f8>−0.055

Rule 7: f1>0.65 AND f2>−0.77 AND f3>17.75 AND f4>−0.34 AND f6>−5.5 AND f7>45.56 AND f8>−0.055 AND f9<=−0.23 AND f10>7.0

Rule 8: f1>0.5 AND f2>−0.77 AND f3>17.75 AND f4>−0.34 AND f6>−5.5 AND f7>45.56 AND f9>−0.23 AND f12<=0.625 AND f13<=0.144401 AND f13>−0.303435 AND f14<=6.8 AND f15<=18.04

Rule 9: f1>0.5 AND f2>−0.77 AND f3>17.75 AND f4>−0.34 AND f6>−5.5 AND f7>45.56 AND f9>−0.23 AND f12<=0.625 AND f13<=0.144401 AND f14>6.8 AND f11<=17.92

Rule 10: f1>0.5 AND f2>−0.77 AND f3>17.75 AND f4>−0.34 AND f6>−5.5 AND f7>45.56 AND f9>−0.23 AND f12<=0.625 AND f13>0.144401

Rules for Rank Ordering of Immunogenic Peptides

TABLE 12
Method of rank ordering immunogenic peptides

| Steps as shown in FIG. 1 | Output from the steps | Score |
|---|---|---|
| TCR binding (Step-10) | Positive by Ensemble model-2 and 3 | 3 |
| | Positive by Ensemble model 3 only | 2 |
| | Positive by Ensemble model-2 only | 1 |
| | Negative by both Ensemble | 0 |
| | model 2 and 3 | |
| MHC binding (IC₅₀) | <=100 nM | 4 |
| (Step-11) | >100 nM, <=500 nM | 3 |
| | >500 nM, <=1000 nM | 2 |
| | >1000 nM | 1 |
| Expression of the mutant | =0 | 0 |
| allele (Step-7) | 1-5 (read count) | 1 |
| | 6-10 (read count) | 2 |
| | 11-50 (read count) | 3 |
| | >50 (read count) | 4 |
| TAP binding (Step-12) | <0.5 | 3 |
| | >=0.5 | 1 |
| Proteasomal cleavage | <10.0 | 1 |
| (Step-13) | >=10 | 3 |

Scores are combined to create a rank ordered score for each peptide.

### Example 2

The example demonstrates an exemplary methodology for predicting immunogenic peptide from a human Head and Neck cancer sample starting from human cancer tissue sample

Exome Sequencing

The exome sequencing was performed for the tumor and normal samples. The exome capturing was performed using Agilent SureSelect Human All Exon V5 kit. The RNA sequencing (RNA-seq) was performed for the total RNA extracted after Ribo-depletion of tumor sample RNA. All paired-end sequencing was performed using Illumina HiSeq 2500 platform. Total data obtained for the exome-seq and RNA-seq sample exceeds 12 Gb and more than 90% of data exceed Q30 (shown in Table 12).

The exome-seq data is first pre-processed, where we remove the low quality reads/bases and adapter sequences. The pre-processed reads is then aligned to the human reference genome (hg19) using BWA program with default parameters. Then, we apply GATK-best practices where we remove the duplicate reads using Picard tools and re-align, re-calibrate using GATK and keep the file ready for somatic mutation identification (Table 13). The somatic mutations in the samples are identified using Strelka program. After this, only the quality passed and on-target mutations are processed further. A total of 222 mutations were identified in this sample. Of these 210 are SNPs and 12 are Indels (Table 14). Of the total coding mutations, 106 of them are of missense type (Table 16).

RNA Sequencing

The RNA-seq data is first pre-processed, where we remove the low quality reads/bases, adapter sequences and unwanted sequences like ribosomal RNA, tRNAs, repeat sequences. The pre-processed reads is then aligned to human reference transcriptome and genome using STAR aligner (Table 17). The expression of the gene is then identified using Cufflinks program.

HLA-Typing

The RNA-seq data is then used for HLA typing [27, 28]. We used Seq2HLA program for HLA typing from RNA-seq. The Class-I HLA alleles identified for this sample is provided in Table 18. The expression of the HLA genes is provided in Table 19. The read depth of the mutant allele in RNA-seq is then calculated. Of the total mutations, we found 62 mutations with read support >=1 in RNA-seq. These mutations are also termed as expressed mutations. The 62 mutations generated 578 unique 9-mer peptides.

Immunogenic Peptide Identification

The peptides derived from the expressed mutations were scored for TCR-binding followed by HLA binding prediction, then TAP prediction and finally proteasomal processing. The immunogenic peptides were further ranked based on the expression level of genes and variants, affinity of HLA binding, sensitivity to proteasomal processing and binding to the transporter. We applied the ranking method to 220 unique immunogenic peptides from this Head and Neck cancer sample. The ranked peptide along with HLA information is provided in Table 20.

TABLE 13
Summary of data generated from head and
neck cancer tumor and paired normal sample

| | Exome-seq | | RNA-seq |
|---|---|---|---|
| Data Metrics | Blood | Tumor | Tumor |
| Total reads | 12, 65, 08, 302 | 12, 38, 71, 688 | 136,893,000 |
| Total data (Gb) | 12.65 | 12.39 | 13.69 |
| Average read length | 100 | 100 | 100 |
| (bp) | | | |
| GC (%) | 48.98 | 49.85 | 54.55 |
| Average base quality | 39.90 | 39.74 | 34.97 |
| (Phred) | | | |
| Total data >= Q30 (%) | 96.91 | 96.39 | 90.62 |

TABLE 14
Preprocessing, alignment and coverage summary of exome
sequencing data

| Data and analysis metrics | Blood | Tumor |
|---|---|---|
| Total reads after | 12, 64, 41, 480 | 12, 38, 71, 678 |
| pre-processing | | |
| Total data after | 12.63 | 12.38 |
| pre-processing (Gb) | | |
| Average read length (bp) | 99.91 | 99.94 |
| Average base quality (Phred) | 39.72 | 39.56 |
| Data >= Q30 (%) | 96.96 | 96.45 |
| after pre-processing | | |
| Total aligned reads | 126,390,638 | 123,793,462 |
| Alignment (%) | 99.96 | 99.94 |
| Duplicate (%) | 14.98 | 16.20 |
| Panel length | 5, 03, 90, 601 | 5, 03, 90, 601 |
| Panel Coverage (%) | 99.85 | 99.84 |
| Panel Ontarget Region | 111.01 | 130.42 |
| Avg. Depth | | |
| On-target (%) | 62.61 | 75.75 |

TABLE 15
Summary of variants detected in the sample

| | |
|---|---|
| Total variants | 222 |
| Total SNPs | 210 |
| Total Indels | 12 |
| Transition SNPs | 136 |
| Transversion SNPs | 74 |
| Ts/Tv | 1.84 |

TABLE 16
Classification of protein-altering variants

| Variant Class | # of mutations |
|---|---|
| Missense | 106 |
| Frameshift | 3 |
| InFrame | 3 |
| Total | 112 |

Missense - Genetic alteration that results in a different amino acid.
Frameshift - Genetic alteration that changes the reading frame. This typically results in a string of different amino acids substitutions before encountering a stop codon.
InFrame - Genetic alteration that results in either deletion or insertion of one or more amino acids.

TABLE 17
Pre-processing and alignment summary of RNA sequence data

| | |
|---|---|
| Read Count After Adapter Trimming | 133,225,190 |
| Read Count After Contamination Removal | 92,623,074 |
| Reads Aligned | 75,489,728 |
| Reads Unaligned | 17,133,346 |
| Reads Aligned % | 81.50 |
| % data lost after Pre-Precessing | 32.34 |

TABLE 18
HLA class I alleles present in the sample

| | |
|---|---|
| HLA-A | HLA-A33:03, HLA-A02:01 |
| HLA-B | HLA-B58:01, HLA-B35:01 |
| HLA-C | HLA-C03:02, HLA-C04:01 |

TABLE 19
Expression of HLA class I genes in the sample

| HLA gene | Gene Expression (RPKM) |
|---|---|
| HLA-A | 657.30 |
| HLA-B | 987.41 |
| HLA-C | 691.26 |

TABLE 20
Rank ordered list of immunogenic peptides from the mutations in
head and neck cancer sample

| | | Amino | Mutant | |
|---|---|---|---|---|
| | | acid | Peptide | |
| Rank | Gene | change | (9mer) | HLA Types |
| 1 | PIK3CA | p.E542K | strdpls(K)i | HLA-B35:01, HLA-A02:01, HLA-B58:01, HLA-C04:01, HLA- |
| | | | | C03:02, HLA-A33:03 |
| 2 | BRPF3 | p.R570W | rllieli(W)k | HLA-B35:01, HLA-A02:01, HLA-B58:01, HLA-C04:01, HLA- |
| | | | | C03:02, HLA-A33:03 |
| 3 | ZBTB6 | p.E196Q | stveslts(Q) | HLA-B35:01, HLA-A02:01, HLA-B58:01, HLA-C04:01, HLA- |
| | | | | C03:02, HLA-A33:03 |
| 3 | BRPF3 | p.R570W | llieli(W)kr | HLA-A33:03 |
| 5 | BRPF3 | p.R570W | lieli(W)kre | HLA-B35:01, HLA-A02:01, HLA-B58:01, HLA-C04:01, HLA- |
| | | | | C03:02, HLA-A33:03 |
| 6 | PIK3CA | p.E542K | (K)iteiqekdf | HLA-B35:01, HLA-A02:01, HLA-B58:01, HLA-C04:01, HLA- |
| | | | | C03:02, HLA-A33:03 |
| 7 | ZBTB6 | p.E196Q | lts(Q)rkemk | HLA-B35:01, HLA-A02:01, HLA-B58:01, HLA-C04:01, HLA- |
| | | | | C03:02, HLA-A33:03 |
| 8 | BRPF3 | p.R570W | llieli(W)kr | HLA-B35:01, HLA-A02:01, HLA-B58:01, HLA-C04:01, HLA- |
| | | | | C03:02 |

### REFERENCES

1. Schumacher, T. N. and R. D. Schreiber, Neoantigens in cancer immunotherapy. Science, 2015. 348(6230): p. 69-74.

2. Gubin, M. M., et al., Tumor neoantigens: building a framework for personalized cancer immunotherapy. J Clin Invest, 2015. 125(9): p. 3413-21.

3. van der Burg, S. H., et al., Vaccines for established cancer: overcoming the challenges posed by immune evasion. Nat Rev Cancer, 2016. 16(4): p. 219-33.

4. Romero, P., et al., The Human Vaccines Project: A roadmap for cancer vaccine development. Sci Transl Med, 2016. 8(334): p. 334ps9.

5. Yadav, M., et al., Predicting immunogenic tumour mutations by combining mass spectrometry and exome sequencing. Nature, 2014. 515(7528): p. 572-6.

6. Vaughan, K., et al., Deciphering the MHC-associated peptidome: a review of naturally processed ligand data. Expert Rev Proteomics, 2017: p. 1-8.

7. Wieczorek, M., et al., Major Histocompatibility Complex (MHC) Class I and MHC Class II Proteins: Conformational Plasticity in Antigen Presentation. Front Immunol, 2017. 8: p. 292.

8. Basler, M., C. J. Kirk, and M. Groettrup, The immunoproteasome in antigen processing and other immunological functions. Curr Opin Immunol, 2013. 25(1): p. 74-80.

9. Eggensperger, S. and R. Tampe, The transporter associated with antigen processing: a key player in adaptive immunity. Biol Chem, 2015. 396(9-10): p. 1059-72.

10. Mahmutefendic, H., et al., Endosomal trafficking of open Major Histocompatibility Class I conformers—implications for presentation of endocytosed antigens, Mol Immunol, 2013. 55(2): p. 149-52.

11. Roche, P. A. and K. Furuta, The ins and outs of MHC class II-mediated antigen processing and presentation. Nat Rev Immunol, 2015. 15(4): p. 203-16.

12. Neches, J., et al., Towards a systems understanding of MHC class I and MHC class II antigen presentation. Nat Rev Immunol, 2011. 11(12): p. 823-36.

13. Leavy, O., Antigen presentation: cross-dress to impress. Nat Rev Immunol, 2011. 11(5): p. 302-3.

14. Joffre, O. P., et al., Cross-presentation by dendritic cells. Nat Rev Immunol, 2012, 12(8): p. 557-69.

15. Branca, M. A., Rekindling cancer vaccines. Nat Biotechnol, 2016. 34(10): p. 1019-1024.

16. Ott, P. A., et al., An immunogenic personal neoantigen vaccine for patients with melanoma. Nature. 2017. 547(7662): p. 217-221.

17. Sahin, U., et al., Personalized RNA mutanome vaccines mobilize poly-specific therapeutic immunity against cancer. Nature, 2017. 547(7662): p. 222-226.

18. Carreno, B. M. and E. R. Mardis, A Vaccine for Cancer? Sci Am, 2016. 314(4): p. 46.

19. Carreno, B. M., et al., Cancer immunotherapy. A dendritic cell vaccine increases the breadth and diversity of melanoma neoantigen-specific T cells. Science, 2015. 348(6236); p. 803-8.

20. Liu, X. S. and E. R. Mardis, Applications of Immunogenomics to Cancer. Cell, 2017. 168(4): p. 600-612.

21. Hundal, J., et al., Cancer Immunogenomics: Computational Neoantigen Identification and Vaccine Design. Cold Spring Harb Symp Quant Biol, 2016, 81: p. 105-111.

22. Turajlic, S., et al., Insertion-and-deletion-derived tumour-specific neoantigens and the immunogenic phenotype: a pan-cancer analysis. Lancet Oncol, 2017. 18(8): p. 1009-1021.

23. Romero Arenas, M. A., et al., Preliminary whole-exome sequencing reveals mutations that imply common tumorigenicity pathways in multiple endocrine neoplasia type 1 patients. Surgery. 2014. 156(6): p. 1351-7; discussion 1357-8.

24. Karosiene, E., et al., NetMHCcons: a consensus method for the major histocompatibility complex class I predictions. Immunogenetics, 2012. 64(3): p. 177-86.

25. Nielsen, M., et al., The role of the proteasome in generating cytotoxic T-cell epitopes: insights obtained from improved predictions of proteasomal cleavage. Immunogenetics. 2005. 57(1-2): p. 33-41.

26. Hall, M. A., Correlation-based Feature Selection for Machine Learning. 1999.

27. Sidney, J., et al., HLA class I supertypes: a revised and updated classification. BMC Immunol, 2008. 9: p. 1.

28. Greenbaum, J., et al., Functional classification of class II human leukocyte antigen (HLA) molecules reveals seven different supertypes and a surprising degree of repertoire sharing across supertypes. Immunogenetics, 2011. 63(6): p. 325-35.

1. A method of selecting mammalian tumor immunogenic peptide(s) from genetically altered protein(s) expressed by a mammalian tumor cell or a mammalian tumor tissue from a subject which comprises:
a) obtaining a sample from the subject:
b) identifying the genetically altered protein(s) expressed by the mammalian tumor cell or the mammalian tumor tissue in the sample through nucleic acid sequence(s) encoding the altered protein(s);
b) producing peptide fragment(s) comprising at least one amino acid mutation from the genetically altered protein(s) so identified in step (a), so as to obtain peptide variant(s) associated with the mammalian tumor cell or the mammalian tumor tissue;
c) selecting the peptide variant(s) from step b, which binds T-cell receptor (TCR) comprising:
i) selecting the peptide variant(s) with a pre-defined length:
ii) characterizing the peptide variant(s) in silico by selecting and matching features associated with an amino acid at each position of the peptide with selected pre-defined features for each position of peptides recognized by TCR associated with either CD8+ T-cell or CD4+ T-cell, so as to obtain predictive ability of the peptide variant(s) to interact with the TCR;
iii) selecting the peptide variant(s) in step c.ii based on predicted ability of the peptide variant(s) to interact with the TCR, so as to be an immunogenic peptide that may or can serve as a mammalian tumor immunogenic peptide(s);
thereby, selecting mammalian tumor immunogenic peptide(s) from genetically altered protein(s) expressed by the mammalian tumor cell or the mammalian tumor tissue.

2. The method of claim 1, where the immunogenic peptide is selected further by its ability to bind MHC class-I or class-II protein(s) comprising:
a) calculating the binding affinity of the immunogenic peptide to MHC class-I or class-II protein(s);
b) further selecting a set of peptide variant(s) from the previous step where the binding affinity of the unmutated or wild-type peptide is weaker than the variant or the mutated peptide for MHC class-I or class-II protein(s).

3. (canceled)

4. The method of claim 1, wherein the immunogenic peptide is further selected by its potential or ability to be produced inside the cell by processes comprising:
a) determining the action of proteases, which are part of the proteasomal or immunoproteasomal complexes, based on the probability that the processing event of the altered protein(s) will produce the immunogenic peptide so selected; and
b) determining the entry of the immunogenic peptide into the endoplasmic reticulum compartment by binding to peptide transporters expressed on the surface of the compartment.

5.-6. (canceled)

7. The method of claim 1, wherein in step (a) identifying the genetically altered protein(s) expressed by the mammalian tumor cell or the mammalian tumor tissue through nucleic acid sequence(s) encoding the altered protein(s) comprises:
a) identifying tumor variants from transcriptome analysis of the mammalian tumor cell or mammalian tumor tissue corresponding to protein coding and protein non-coding sequences; and
b) performing conceptual translation or in silico translation of the coding sequences in step (a) so as to identify the genetically altered protein(s) expressed by the mammalian tumor cell or the mammalian tumor tissue.

8. The method of claim 7, wherein in step (a) identifying tumor variants from transcriptome analysis of the mammalian tumor cell or mammalian tumor tissue comprises
a) determining nucleotide sequence of transcripts produced by the mammalian tumor cell or mammalian tumor tissue; and
b) comparing the determined nucleotide sequence of transcripts in (a) with a reference nucleotide sequence of transcripts produced by mammalian non-tumor cell or mammalian non-tumor tissue, so as to identify nucleotide sequence changes in the protein coding and protein non-coding sequences;
thereby, identifying tumor variants from transcriptome analysis of the mammalian tumor cell or mammalian tumor tissue.

9.-12. (canceled)

13. The method of claim 1, wherein in step (b) producing peptide fragment(s) comprising at least one amino acid mutation from each genetically altered protein, so as to obtain peptide variant(s) associated with the mammalian tumor cell or the mammalian tumor tissue comprises:
a) defining length of the peptide fragment(s) to be produced from the genetically altered protein; and
b) producing in silico peptide fragment(s) of the pre-defined length at a site of alteration in the protein comprising at least one mutated amino acid of the genetically altered protein.

14. The method of claim 1, wherein the length of the peptide fragment(s) to be produced from the genetically altered protein or the peptide fragment(s) of the pre-defined length is 8 amino acids or more.

15. The method of claim 1, wherein the length of the peptide fragment(s) to be produced from the genetically altered protein or peptide fragment(s) of the pre-defined length is less than 18 amino acids.

16. The method of claim 1, wherein the length of the peptide fragment(s) to be produced from the genetically altered protein or the peptide fragment(s) of the pre-defined length is 9 amino acids long.

17. The method of claim 16, wherein the length of the peptide fragment(s) to be produced from the genetically altered protein or the peptide fragment(s) of the pre-defined length further supports interaction with the TCR of CD8+ T-cell or CD4+ T-cell.

18.-24. (canceled)

25. The method of claim 1, wherein obtaining the pre-defined features for each position of peptides recognized by TCR-associated with either CD8+ T-cell or CD4+ T-cell comprises
a) aligning end-to-end peptides of same size with pre-defined length known to be bound by TCR-associated with either CD8+ T-cell or CD4+ T-cell;
b) optionally, aligning end-to-end peptides of same size as in (a) known not to be bound by TCR-associated with either CD8+ T-cell or CD4+ T-cell but known to be bound by either MHC class I protein(s) or MHC class II protein(s); and
c) determining amino acid features most prevalent or avoided at each amino acid position from the aligned sequences in (a) and/or (b);
thereby, obtaining the pre-defined features for each position of peptides recognized by TCR-associated with either CD8+ T-cell or CD4+ T-cell.

26.-27. (canceled)

25. The methods of claim 1, further comprising
predicting a rank ordered list of the immunogenic peptides derived from mammalian tumor cell or mammalian tumor tissue so selected, wherein the peptide is a peptide variant and wherein rank ordering peptides is based on a combination of the following parameters:
a) expression of variant gene from which variant peptide is derived;
b) predicted ability to bind TCR of CD8+ T-cell;
c) binding affinity of the peptide to MHC class-I protein(s);
d) peptide processing by immunoproteosomes or proteasomes;
e) peptide transporter binding; and
wherein each parameter may be subdivided to reflect quality of the parameter through numerical value(s) or range(s) of values, and wherein the numerical value(s) or range(s) of values from the parameters assessed or combined so as to produce output(s) permissive of sorting by ascending or descending order, thereby predicting a rank ordered list of the immunogenic peptides derived from mammalian tumor cell or mammalian tumor tissue so selected.

29.-41. (canceled)

42. A method of preparing a subject-specific immunogenic peptide composition comprising selecting cancer immunogenic peptides from genetically altered proteins expressed by mammalian cancer cells and tissues by the method of claim 1 thereby preparing the subject-specific immunogenic composition.

43.-48. (canceled)

49. A method of selecting cross species cancer vaccines from genetically altered proteins expressed by mouse and human cancer cells and tissues which comprises:
a. calculating the probability of HLA binding with optimal processing sites from a library of mutant cancer peptides;
b. calculating the probability of TCR binding to generate a T-cell response
c. selecting the mutant cancer peptides having the highest probability so calculated from step (a) and (b) that can modulate the immune response of a mouse and a human, when challenged with the mutant cancer peptide thereby selecting cross species cancer vaccines; wherein the mouse and human subjects carry the same mutation and express the same HLA molecule that binds the mutant cancer peptide.

50.-58. (canceled)

59. A method of treating a cancer in a subject in need thereof comprising:
a) obtaining a sample from the subject;
b) identifying the genetically altered protein(s) expressed by the mammalian tumor cell or the mammalian tumor tissue in the sample through nucleic acid sequence(s) encoding the altered protein(s);
b) producing peptide fragment(s) comprising at least one amino acid mutation from the genetically altered protein(s) so identified in stop (a), so as to obtain peptide variant(s) associated with the mammalian tumor cell or the mammalian tumor tissue;
c) selecting the peptide variant(s) from step b, which binds T-cell receptor (TCR) comprising:
i) selecting the peptide variant(s) with a pre-defined length;
ii) characterizing the peptide variant(s) in silico by selecting and matching features associated with an amino acid at each position of the peptide with selected pre-defined features for each position of peptides recognized by TCR associated with CD8+ T-cell or CD4+ T-cell, so as to obtain predictive ability of the peptide variant(s) to interact with the TCR:
iii) selecting the peptide variant(s) in step c.ii based on predicted ability of the peptide variant(s) to interact with the TCR, so as to be an immunogenic peptide or alternatively whose sequence forms a basis for a mammalian tumor vaccine(s):
d) forming a vaccine comprising a peptide with the sequence of at least one immunogenic peptide so selected; and
e) administering the vaccine in an effective amount to the subject so as to treat the cancer in the subject.

60. The method of claim 1, wherein the peptide variant(s) with a pre-defined length is 9 amino acid long and pre-defined features comprise one or more of polar, non-polar, hydrophobic, helix/turn motif, β-sheet structure motif, charge of main chain, charge of side chain, solvent accessibility of an amino acid, spatial flexibility of the main chain and spatial flexibility of side chain of an amino acid.
